(19) United States
(12) Patent Application Publication
(10) Pub. No.: US 2023/0118587 A1
(43) Pub. Date: Apr. 20, 2023

(54) Lentiviral Vectors

(71) Applicant: Oxford BioMedica (UK) Limited, Oxford (GB)

(72) Inventor: Jordan Wright, Oxford (GB)

(21) Appl. No.: 17/911,115

(22) PCT Filed: Mar. 11, 2021
(86) PCT No.: PCT/GB2021/050620

§ 371 Date: Sep. 12, 2022

(30) Foreign Application Priority Data
Mar. 13, 2020 (GB) 2003709.9
Mar. 13, 2020 (GB) 2003710.7
Mar. 13, 2020 (GB) 2003711.5

(51) Int. Cl. C12N 15/86

(52) U.S. Cl. CPC C12N 15/86; C12N 2740/15043; C12N 2740/15052; C12N 2740/15022

(57) ABSTRACT

The present invention provides a lentiviral vector genome comprising at least one modified viral cis-acting sequence, wherein at least one internal open reading frame (ORF) in the viral cis-acting sequence is disrupted.

### FIELD OF THE INVENTION

The invention relates to lentiviral vectors and their production. More specifically, the present invention relates to a lentiviral vector genome comprising a modified viral cis-acting sequence, wherein at least one internal open reading frame (ORF) in the viral cis-acting sequence is disrupted. The present invention also provides a lentiviral vector genome lacking either (i) a nucleotide sequence encoding Gag-p17 or (ii) a fragment of a nucleotide sequence encoding Gag-p17. Methods and uses involving such a lentiviral vector genome are also encompassed by the invention.

### BACKGROUND TO THE INVENTION

The development and manufacture of viral vectors towards vaccines and human gene therapy over the last several decades is well documented in scientific journals and in patents. The use of engineered viruses to deliver transgenes for therapeutic effect is wide-ranging. Contemporary gene therapy vectors based on RNA viruses such as γ-retroviruses and lentiviruses (Muhlebach, M. D. et al., 2010, Retroviruses: Molecular Biology, Genomics and Pathogenesis, 13:347-370; Antoniou, M. N., Skipper, K. A. & Anakok, O., 2013, Hum. Gene Ther., 24:363-374), and DNA viruses such as adenovirus (Capasso, C. et al., 2014, Viruses, 6:832-855) and adeno-associated virus (AAV) (Kotterman, M. A. & Schaffer, D. V., 2014, Nat. Rev. Genet., 15:445-451) have shown promise in a growing number of human disease indications. These include ex vivo modification of patient cells for hematological conditions (Morgan, R. A. & Kakarla, S., 2014, Cancer J., 20:145-150; Touzot, F. et al., 2014, Expert Opin. Biol. Ther., 14:789-798), and in vivo treatment of ophthalmic (Balaggan, K. S. & Ali, R. R., 2012, Gene Ther., 19:145-153), cardiovascular (Katz, M. G. et al., 2013, Hum. Gene Ther., 24:914-927), neurodegenerative diseases (Coune, P. G., Schneider, B. L. & Aebischer, P., 2012, Cold Spring Harb. Perspect. Med., 4:a009431) and tumor therapy (Pazarentzos, E. & Mazarakis, N. D., 2014, Adv. Exp. Med Biol., 818:255-280).

As the successes of these approaches in clinical trials continues towards regulatory approval and commercialisation, safety aspects involved in administration of viral vectors to patients, for example in the context of vaccination and gene therapy, are of particular importance.

There is an ongoing need in the art for viral vectors with improved safety profiles.

In addition, the size of the transgenes used for therapeutic effect is increasing. Therefore, there is an ongoing need in the art to increase the capacity of the viral vectors, i.e. the size of the transgene (or payload) that the viral vector can deliver.

### SUMMARY OF THE INVENTION

In one aspect, the present invention is based on the disruption of internal open reading frames (ORFs) within a viral cis-acting sequence, for example the Rev response element (RRE), present within lentiviral vector genomes. The present inventors have surprisingly found that modifications in viral cis-acting sequence to disrupt at least one internal ORF, for example by mutating the ATG sequence which denotes the start of the at least one internal ORF, are tolerated such that the modified viral cis-acting sequence retains its function, for example the modified RRE retains Rev binding capacity.

Accordingly, in one aspect the present invention provides a lentiviral vector genome comprising at least one modified viral cis-acting sequence, wherein at least one internal open reading frame (ORF) in the viral cis-acting sequence is disrupted. The at least one internal ORF may be disrupted by mutating at least one ATG sequence (ATG sequences may function as translation initiation codons).

In some embodiments, the at least one viral cis-acting sequence is:
a) a Rev response element (RRE); and/or
b) a Woodchuck hepatitis virus (WHV) post-transcriptional regulatory element (WPRE).

In some embodiments, the at least one viral cis-acting sequence is a RRE.

In some embodiments, the at least one viral cis-acting sequence is a WPRE.

In some embodiments, the lentiviral vector genome comprises a modified RRE as described herein and a modified WPRE as described herein.

In a further aspect, the present invention provides a lentiviral vector genome comprising a modified Rev response element (RRE), wherein at least one internal open reading frame (ORF) in the RRE is disrupted. The at least one internal ORF may be disrupted by mutating at least one ATG sequence (ATG sequences may function as translation initiation codons).

In a further aspect, the present invention provides a lentiviral vector genome comprising a modified Woodchuck hepatitis virus (WHV) post-transcriptional regulatory element (WPRE), wherein at least one internal open reading frame (ORF) in the WPRE is disrupted. The at least one internal ORF may be disrupted by mutating at least one ATG sequence.

In a further aspect, the present invention is based on the deletion of the nucleotide sequence encoding Gag-p17. The present inventors have surprisingly found that the deletion of the nucleotide sequence encoding Gag-p17 from the backbone of the lentiviral vector genome does not significantly impact vector titres during lentiviral vector production.

Accordingly, in a further aspect the present invention provides a lentiviral vector genome lacking either (i) a nucleotide sequence encoding Gag-p17 or (ii) a fragment of a nucleotide sequence encoding Gag-p17. The lentiviral vector genome may, for example, not express Gag-p17 or a fragment thereof.

In some embodiments, the lentiviral vector genome comprises at least one modified viral cis-acting sequence, wherein at least one internal ORF in the viral cis-acting sequence is disrupted. The at least one viral cis-acting sequence may be a RRE. The at least one viral cis-acting sequence may be a WPRE. In some embodiments, the lentiviral vector genome comprises a modified RRE and a modified WPRE, wherein at least one internal ORF in the RRE is disrupted and at least one internal ORF in the WPRE is disrupted. The at least one internal ORF may be disrupted by mutating at least one ATG sequence.

In some embodiments, the modified RRE comprises less than eight ATG sequences.

In a further aspect the present invention provides a lentiviral vector genome comprising a modified Rev response element (RRE), wherein the modified RRE comprises less than eight ATG sequences.

In some embodiments, the RRE is a full-length RRE. In some embodiments, the RRE is a minimal RRE.

In some embodiments, the RRE comprises:
a) a sequence having at least 80% identity to SEQ ID NO: 1; and/or
b) a sequence having at least 80% identity to SEQ ID NO: 2.

In some embodiments, the modified RRE comprises the sequence as set forth in SEQ ID NO: 1 or SEQ ID NO: 2, or a sequence having at least 80% identity thereto, wherein at least one ATG sequence selected from the group (a)-(h) is mutated:
a) ATG corresponding to positions 27-29 of SEQ ID NO: 2;
b) ATG corresponding to positions 192-194 of SEQ ID NO: 2;
c) ATG corresponding to positions 207-209 of SEQ ID NO: 2;
d) ATG corresponding to positions 436-438 of SEQ ID NO: 2;
e) ATG corresponding to positions 489-491 of SEQ ID NO: 2;
f) ATG corresponding to positions 571-573 of SEQ ID NO: 2;
g) ATG corresponding to positions 599-601 of SEQ ID NO: 2;
h) ATG corresponding to positions 663-665 of SEQ ID NO: 2.

In some embodiments, the modified RRE comprises less than seven, less than six, less than five, less than four, less than three, less than two or less than one ATG sequence(s).

In some embodiments, the modified WPRE comprises less than seven ATG sequences. In some embodiments, the modified WPRE comprises less than six ATG sequences.

In a further aspect the present invention provides a lentiviral vector genome comprising a modified Woodchuck hepatitis virus (WHV) post-transcriptional regulatory element (WPRE), wherein the modified WPRE comprises less than seven ATG sequences.

In a further aspect the present invention provides a lentiviral vector genome comprising a modified Woodchuck hepatitis virus (WHV) post-transcriptional regulatory element (WPRE), wherein the modified WPRE comprises less than six ATG sequences.

In some embodiments, WPRE comprises:
a) a sequence having at least 80% identity to SEQ ID NO: 11; and/or
b) a sequence having at least 80% identity to SEQ ID NO: 12.

In some embodiments, the modified WPRE may comprise the sequence as set forth in SEQ ID NO: 11 or SEQ ID NO: 12, or a sequence having at least 80% identity (suitably at least 85%, at least 90%, at least 91%, at least 92%, at least 93%, at least 94%, at least 95%, at least 96%, at least 97%, at least 98%, at least 99%) thereto, wherein at least one ATG sequence selected from the group (a)-(g) is mutated:
a) ATG corresponding to positions 53-55 of SEQ ID NO: 11;
b) ATG corresponding to positions 72-74 of SEQ ID NO: 11;
c) ATG corresponding to positions 91-93 of SEQ ID NO: 11;
d) ATG corresponding to positions 104-106 of SEQ ID NO: 11;
e) ATG corresponding to positions 121-123 of SEQ ID NO: 11;
f) ATG corresponding to positions 170-172 of SEQ ID NO: 11; and/or
g) ATG corresponding to positions 411-413 of SEQ ID NO: 11.

In some embodiments, the modified WPRE comprises less than seven, less than six, less than five, less than four, less than three, less than two or less than one ATG sequence(s),In some embodiments, the lentiviral vector genome comprises a modified nucleotide sequence encoding gag, wherein at least one internal ORF in the modified nucleotide sequence encoding gag is disrupted. The at least one internal ORF in the modified nucleotide sequence encoding gag may be disrupted by mutating at least one ATG sequence.

In some embodiments, the nucleotide sequence encoding gag comprises a sequence having at least 80% identity to SEQ ID NO: 6 or SEQ ID NO: 7.

In some embodiments, the modified nucleotide sequence encoding gag comprises less than three ATG sequences (e.g. less than two or less than one internal ATG sequences).

In some embodiments, the lentiviral vector genome lacks either (i) a nucleotide sequence encoding Gag-p17 or (ii) a fragment of a nucleotide sequence encoding Gag-p17. The lentiviral vector genome may, for example, not express Gag-p17 or a fragment thereof.

In some embodiments, the major splice donor site in the lentiviral vector genome is inactivated. The cryptic splice donor site 3′ to the major splice donor site may also be inactivated.

In some embodiments, the lentiviral vector genome further comprises a nucleotide of interest, which may give rise to a therapeutic effect.

In some embodiments, the lentiviral vector genome further comprises a tryptophan RNA-binding attenuation protein (TRAP) binding site

In some embodiments, the lentiviral vector is derived from HIV-1, HIV-2, SIV, FIV, BIV, EIAV, CAEV or Visna lentivirus.

In some embodiments, the lentiviral vector genome is an RNA genome of a lentiviral vector.

In a further aspect, the present invention provides a lentiviral vector comprising the lentiviral vector genome of the invention. The lentiviral vector may be derived from HIV-1, HIV-2, SIV, FIV, BIV, EIAV, CAEV or Visna lentivirus.

In some embodiments, the lentiviral vector of the invention is a transgene expression cassette.

In a further aspect, the present invention provides a nucleotide sequence encoding the lentiviral vector genome of the invention.

In some embodiments, the lentiviral vector genome of the invention may be suitable for use in a lentiviral vector in a U3 or tat-independent system for vector production. As described herein, 3^{rd} generation lentiviral vectors are U3/tat-independent, and the lentiviral vector genome according to the present invention may be used in the context of a 3^{rd} generation lentiviral vector. In one embodiment, tat is not provided in the lentiviral vector production system, for example tat is not provided in trans. In one embodiment, the cell or vector or vector production system as described herein does not comprise the tat protein. In one embodiment, HIV-1 U3 is not present in the lentiviral vector production system, for example HIV-1 U3 is not provided in cis to drive transcription of the vector genome expression cassette.

In some embodiments, the lentiviral vector genome of the invention:
a) is for use in a tat-independent lentiviral vector;
b) is produced in the absence of tat;
c) has been transcribed independently of tat;
d) for use in a U3-independent lentiviral vector;
e) has been transcribed independently of the U3 promoter; or
f) has been transcribed by a heterologous promoter.

In some embodiments, transcription of the nucleotide sequence as described herein is not dependent on the presence of U3. The nucleotide sequence may be derived from a U3-independent transcription event. The nucleotide sequence may be derived from a heterologous promoter. A nucleotide sequence as described herein may not comprise a native U3 promoter.

In a further aspect, the present invention provides an expression cassette comprising the nucleotide sequence of the invention.

In a further aspect, the present invention provides a viral vector production system comprising a set of nucleotide sequences, wherein the nucleotide sequences encode vector components including gag-pol, env, optionally rev, and the lentiviral vector genome of the invention.

In a further aspect, the present invention provides a cell comprising the lentiviral vector genome of the invention, the nucleotide sequence of the invention, the expression cassette of the invention or the viral vector production system of the invention.

In a further aspect, the present invention provides a cell for producing lentiviral vectors comprising:
a) nucleotide sequences encoding vector components including gag-pol and env, and optionally rev, and a nucleotide sequence of the invention or the expression cassette of the invention; or
b) the viral vector production system of the invention; and
c) optionally a nucleotide sequence encoding a modified U1 snRNA and/or optionally a nucleotide sequence encoding TRAP.

In a further aspect, the present invention provides a method for producing a lentiviral vector, comprising the steps of:

(i) introducing:
a) nucleotide sequences encoding vector components including gag-pol and env, and optionally rev, and a nucleotide sequence of the invention or the expression cassette of the invention; or
b) the viral vector production system of the invention; and
c) optionally a nucleotide sequence encoding a modified U1 snRNA and/or optionally a nucleotide sequence encoding TRAP into a cell;
   (ii) optionally selecting for a cell which comprises the nucleotide sequences encoding vector components and the lentiviral vector genome; and
   (iii) culturing the cell under conditions suitable for the production of the lentiviral vector.

In a further aspect, the present invention provides a lentiviral vector produced by the method of the invention.

In a further aspect, the present invention provides the use of the lentiviral vector genome of the invention, the nucleotide sequence of the invention, the expression cassette of the invention, the viral vector production system of the invention, or the cell of the invention for producing a lentiviral vector.

In a further aspect, the present invention provides a nucleotide sequence comprising the modified RRE as described herein.

In a further aspect, the present invention provides a cell transduced by the lentiviral vector genome of the invention or the lentiviral vector of the invention.

In a further aspect, the present invention provides a pharmaceutical composition comprising the lentiviral vector as described herein or a cell or tissue transduced with the viral vector as described herein, in combination with a pharmaceutically acceptable carrier, diluent or excipient.

In a further aspect, the present invention provides a pharmaceutical composition for treating an individual by gene therapy, wherein the composition comprises a therapeutically effective amount of a lentiviral vector.

In a further aspect, the present invention provides a lentiviral vector of the invention or a cell or tissue transduced with the lentiviral vector of the invention for use in therapy.

In a further aspect, the present invention provides the use of a lentiviral vector of the invention, a production cell of the invention or a cell or tissue transduced with the lentiviral vector of the invention for the preparation of a medicament to deliver a nucleotide of interest to a target site in need of the same.

### DESCRIPTION OF THE FIGURES

FIG. 1

A schematic showing the removal of ORFs in the RRE-cppt region within lentiviral vector genomes. The upper panel displays the typical configuration of a third-generation lentiviral vector expression cassette (RNA is encoded from the 5′R region). Key: RU5 (LTR regions), ψ (core packaging signal), MSD (major splice donor), gag (retained sequence from the gag ORF as part of the broader packaging signal), RRE (rev response element), sa7 (HIV-1 splice acceptor 7), cppt (central polypurine tract), Pro (promoter), NOI (nucleotide of interested e.g. transgene ORF), PRE (post-transcriptional regulatory element), ppt (polypurine tract). Expanded below this is a focused view of all the sub-ORFs retained within the RRE-cppt sequence in different frames; the cppt also harbours an ATG codon and sub-ORF from the Pol gene. All ATG sequences and their respective modifications are indicated. In standard contemporary lentiviral vector genomes, the RRE (˜800 nts) has essentially been repositioned from the 3′end of the wild type HIV-1 genome, where in its native context it overlaps the envelope ORF. Until the present invention, no attempts have been reported to remove the remaining envelope partial ORF, which conceivably could be translated from the vector genome RNA (‘STD-RREcppt’ is the typical sequence found within standard lentiviral vector genomes. The variants ‘RRE[6-ATGKO]’ and ‘RRE[8-ATGKO]’ both contain mutations such that all sub-ORFs of >7 residues have been ablated, including the cppt region; however, RRE[8-ATGKO] also contains mutations to ablate the two sub-ORFs of 7 residues. The novel variant Thin-RRE[2-ATGKO]′ (also harbours the cppt sub-ORF ablation) is a truncated version of the RRE of only 234nt, and is also ablated for sub-ORFs.

FIG. 2

A RRE variant modified to lack all sub-ORFs of >7 residue is fully functional. Lentiviral vectors encoding GFP were produced in serum-free, suspension HEK293T cells by transient transfection and titrated by flow cytometry of transduced cells. Standard vector (STDRREcppt) was produced alongside a vector containing variant RRE[6-ATGKO] and a vector completely lacking the RRE (but retaining the cppt). The data demonstrate that the variant lacking the sub-ORFs in RRE and cppt was fully functional despite the array of inserted nucleotides ablating the ATG codons.

FIG. 3

A RRE variant modified to lack all sub-ORFs of >7 residue is fully functional in the presence of modified U1 snRNA. Lentiviral vectors encoding GFP were produced in serum-free, suspension HEK293T cells by transient transfection and titrated by flow cytometry of transduced cells. Standard vector (STDRREcppt) was produced alongside a vector containing variant RRE[6-ATGKO], and modified U1 snRNA targeted to the vector genome RNA was optionally co-transfected (−/+256U1). It has been shown that modified U1 snRNA targeted to the vector genome RNA can lead to titre increase by stabilising the RNA. The data demonstrate that the novel variant lacking the sub-ORFs in RRE and cppt was fully functional, and remained responsive to titre enhancement by modified U1 snRNA, indicating that similar levels of vRNA were generated between the standard and novel RRE elements.

FIG. 4

A schematic showing the removal of ORFs in the gag region of the packaging sequence within lentiviral vector genomes. The upper panel displays the typical configuration of a third-generation lentiviral vector expression cassette (RNA is encoded from the 5′R region). Key: RU5 (LTR regions), ψ (core packaging signal), MSD (major splice donor), gag (retained sequence from the gag ORF as part of the broader packaging signal), RRE (rev response element), sa7 (HIV-1 splice acceptor 7), cppt (central polypurine tract), Pro (promoter), NOI (nucleotide of interested e.g. transgene ORF), PRE (post-transcriptional regulatory element), ppt (polypurine tract). In standard contemporary lentiviral vector genomes, efforts have been made to limit potential gag expression by introducing (typically) a frame shift mutation (+CG) ˜45nt downstream of the primary Gag ATG, resulting in a short ORF of 21 residues, of which the first 15 derive from Gag. Expanded below this is a focused view of all the sub-ORFs retained within gag in different frames, as well as the p17 instability element (p17-INS). All ATG sequences and their respective modifications are indicated. Standard contemporary lentiviral vector genomes harbour the ‘STD-Gag’ configuration, which retains the p17-INS. The modifications to novel variants ‘ΔGag[3-ATGKO]’ and the ‘ΔGag[2-ATGKO]ΔINS’ are shown. Note that the ΔGag[2-ATGKO]ΔINS sequence lacks the entirety of the p17-INS sequence, including the ATG3, and therefore provides ˜250 nt of extra transgene capacity.

FIG. 5

A Gag sequence lacking sub-ORFs and deleted in the p17-INS can functionally replace the Gag sequence of the packaging region of lentiviral vectors. Lentiviral vectors encoding GFP were produced in serum-free, suspension HEK293T cells by transient transfection and titrated by flow cytometry of transduced cells. Standard vector (STD-Gag+STDRREcppt) was produced alongside a vector containing the RRE[6-ATGKO] and the ‘ΔAGag[2-ATGKO]ΔINS’ novel variant sequences, and modified U1 snRNA targeted to the vector genome RNA was optionally co-transfected (−/+256U1). It has been shown that modified U1 snRNA targeted to the vector genome RNA can lead to titre increase by stabilising the RNA. In addition, these comparisons were performed within standard lentiviral vectors harbouring the native major splice donor (wtMSD) or a MSD-mutant (2KO-m5). It has been shown that the MSD generates ‘aberrant’ splice products into the vector genome, leading to less full length, packageable vRNA; provision of modified U1 sRNA can restore titres of MSD-mutated lentiviral vector. The data demonstrate that the variant lacking the sub-ORFs in gag and a deletion in p17-INS can be paired with the RRE variant (also lacking sub-ORFs >7 residues), whilst retaining vector function/titres. The MSD-mutated variants (2KO-m5) also remained responsive to titre enhancement by modified U1 snRNA, indicating that similar levels of vRNA were generated between the standard and novel gag-RREcppt elements.

FIG. 6

A schematic showing the removal of ORFs in the wPRE within lentiviral vector genomes. The upper panel displays the typical configuration of a third-generation lentiviral vector expression cassette (RNA is encoded from the 5′R region). Key: RU5 (LTR regions), ψ (core packaging signal), MSD (major splice donor), gag (retained sequence from the gag ORF as part of the broader packaging signal), RRE (rev response element), sa7 (HIV-1 splice acceptor 7), cppt (central polypurine tract), Pro (promoter), NOI (nucleotide of interested e.g. transgene ORF), PRE (post-transcriptional regulatory element), ppt (polypurine tract). In standard contemporary lentiviral vector genomes, the wPRE X-protein has typically been ablated. However, no attempt has been made to ablate the other sub-ORFs, the most significant of which is a ˜160 residue ORF derived from WHV Pol (the RNAseH domain). Expanded below this is a focused view of all the sub-ORFs retained within the ‘standard’ were the X-protein is ablated (wPRE[X-KO]), and the variant ‘wPREΔORF’ wherein additionally all the remaining 6 ATG codons have been ablated.

FIG. 7

A wPRE variant modified to lack all sub-ORFs is fully functional. Lentiviral vectors encoding GFP were produced in serum-free, suspension HEK293T cells by transient transfection and titrated by flow cytometry of transduced cells. Standard vector (wPRE[X-KO]) was produced alongside a vector containing variant wPREΔORF and a vector completely lacking a wPRE. The data demonstrate that the variant lacking the sub-ORFs in the wPRE was surprisingly fully functional despite the array of inserted nucleotides ablating the ATG codons.

FIG. 8

A schematic of a U1 snRNA molecule and an example of how to modify the targeting sequence. The endogenous non-coding RNA, U1 snRNA binds to the consensus splice donor site (5′-MAGGURR-3′) via the 5′-(AC)UUACCUG-3′ (grey highlighted) native splice donor targeting sequence during early steps of intron splicing. Stem loop I binds to U1A-70K protein that has been shown to be important for polyA suppression. Stem loop II binds to U1A protein, and the 5′-AUUUGUGG-3′ sequence binds to Sm proteins, which together with Stem loop IV, is important for U1 snRNA processing. In the disclosure, the modified U1 snRNA may be modified to introduce a heterologous sequence that is complementary to a target sequence within the vector genome vRNA molecule at the site of the native splice donor targeting sequence; in this figure the example given directs the modified U1 snRNA to 15 nucleotides (256-270 relative to the first nucleotide of the vector genome molecule, 256U1) of a standard HIV-1 lentiviral vector genome (located in the SL1 loop if the packaging signal).

### DETAILED DESCRIPTION OF THE INVENTION

Viral Cis-Acting Sequences

The present invention provides a lentiviral vector genome comprising at least one modified viral cis-acting sequence, wherein at least one internal open reading frame (ORF) in the viral cis-acting sequence is disrupted. The at least one internal ORF may be disrupted by mutating at least one ATG sequence (ATG sequences may function as translation initiation codons).

ORFs present in the vector backbone delivered in transduced (e.g. patient) cells could be transcribed, for example, when read-through transcription from upstream cellular promoters occurs (lentiviral vectors target active transcription sites), leading to potential aberrant transcription of genetic material located in the vector backbone in patient cells. This potential aberrant transcription of genetic material located in the vector backbone following read-through transcription could also occur during lentiviral vector production in production cells.

The at least one viral cis-acting sequence present within lentiviral vector genomes may contain multiple internal ORFs. These internal ORFs may be found between an internal ATG sequence of the viral cis-acting sequence and the stop codon immediately 3′ to the ATG sequence.

The present inventors have surprisingly found that modifications in a viral cis-acting sequence to disrupt at least one internal ORF, for example by mutating the ATG sequence which denotes the start of the at least one internal ORF, are tolerated. Thus, the modified viral cis-acting sequence described herein retains its function.

In some embodiments, the lentiviral vector genome comprises at least two (suitably at least three, at least four, at least five, at least six, at least seven) modified viral cis-acting sequences.

In some embodiments, at least two (suitably at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen or at least twenty) internal ORFs in the at least one viral cis-acting sequence may be disrupted. In some embodiments, at least three internal ORFs in the at least one viral cis-acting sequence may be disrupted.

In some embodiments, one (suitably, two, three, four, five, six, seven, eight, nine, ten, eleven, twelve, thirteen, fourteen, fifteen, sixteen, seventeen, eighteen, nineteen or twenty) internal ORFs in the at least one viral cis-acting sequence may be disrupted.

In some embodiments, the at least one internal ORF may be disrupted such that the internal ORF is not expressed. In some embodiments, the at least one internal ORF may be disrupted such that the internal ORF is not translated. In some embodiments, the at least one internal ORF may be disrupted such that no protein is expressed from the internal ORF. In some embodiments, the at least one internal ORF may be disrupted such that no protein is translated from the internal ORF. Thus, the at least one internal ORF present in the modified viral cis-acting sequence in the vector backbone delivered in transduced cells may be disrupted such that aberrant transcription of the internal ORF is prevented when there is read-through transcription from upstream cellular promoters.

In one embodiment, the at least one internal ORF may be disrupted by mutating at least one ATG sequence. A “mutation” of an ATG sequence may comprise one or more nucleotide deletions, additions, or substitutions.

In one embodiment, the at least one ATG sequence may be mutated in the modified viral cis-acting sequence to a sequence selected from the group consisting of:
a) an ATTG sequence;
b) an ACG sequence;
c) an A-G sequence;
d) an AAG sequence;
e) a TTG sequence; and/or
f) an ATT sequence.

The at least one ATG sequence may be mutated to an ATTG sequence in the modified viral cis-acting sequence. The at least one ATG sequence may be mutated to an ACG sequence in the modified viral cis-acting sequence. The at least one ATG sequence may be mutated to an A-G sequence in the modified viral cis-acting sequence. The at least one ATG sequence may be mutated to an AAG sequence in the modified viral cis-acting sequence. The at least one ATG sequence may be mutated to a TTG sequence in the modified viral cis-acting sequence. The at least one ATG sequence may be mutated to an ATT sequence in the modified viral cis-acting sequence.

In one embodiment, the at least one modified viral cis-acting element may lack ATG sequences.

In some embodiments, all ATG sequences within viral cis-acting sequences in the lentiviral vector genome are mutated.

Lentiviral vectors typically comprise multiple viral cis-acting sequences. Example viral cis-acting sequences include the Rev response element (RRE), central polypurine tract (cppt) and Woodchuck hepatitis virus (WHV) post-transcriptional regulatory element (WPRE).

In some embodiments, the at least one viral cis-acting sequence may be at least one lentiviral cis-acting sequence. Example lentiviral cis-acting sequences include the RRE and cppt.

In some embodiments, the at least one viral cis-acting sequence may be at least one non-lentiviral cis-acting sequence.

In some embodiments, the at least one viral cis-acting sequence may be at least one lentiviral cis-acting sequence and at least one non-lentiviral cis-acting sequence.

In some embodiments, the at least one viral cis-acting sequence is:
a) a Rev response element (RRE); and/or
b) a Woodchuck hepatitis virus (WHV) post-transcriptional regulatory element (WPRE).

In some embodiments, the at least one viral cis-acting sequence is a RRE.

In some embodiments, the at least one viral cis-acting sequence is a WPRE.

In some embodiments, the lentiviral vector genome comprises at least two (suitably, at least 3, at least 4, at least 5) modified viral cis-acting sequences.

In some embodiments, the lentiviral vector genome comprises a modified RRE as described herein and a modified WPRE as described herein.

The lentiviral vector genome may further comprise a modified nucleotide sequence encoding gag, wherein at least one internal ORF in the modified nucleotide sequence encoding gag is disrupted. The at least one internal ORF may be disrupted such that the internal ORF is not expressed. Thus, the at least one internal ORF present in the modified nucleotide sequence encoding gag in the vector backbone delivered in transduced cells may be disrupted such that aberrant transcription of the internal ORF is prevented when there is read-through transcription from upstream cellular promoters.

The at least one internal ORF in the modified nucleotide sequence encoding gag may be disrupted by mutating at least one ATG sequence. A “mutation” of an ATG sequence may comprise one or more nucleotide deletions, additions, or substitutions.

In one embodiment, the at least one ATG sequence may be mutated in the modified nucleotide sequence encoding gag to a sequence selected from the group consisting of:
a) an ATTG sequence;
b) an ACG sequence;
c) an A-G sequence;
d) an AAG sequence;
e) a TTG sequence; and/or
f) an ATT sequence

The at least one ATG sequence may be mutated to an ATTG sequence in the modified nucleotide sequence encoding gag. The at least one ATG sequence may be mutated to an ACG sequence in the modified nucleotide sequence encoding gag. The at least one ATG sequence may be mutated to an A-G sequence in the modified nucleotide sequence encoding gag. The at least one ATG sequence may be mutated to an AAG sequence in the modified nucleotide sequence encoding gag. The at least one ATG sequence may be mutated to a TTG sequence in the modified nucleotide sequence encoding gag. The at least one ATG sequence may be mutated to an ATT sequence in the modified nucleotide sequence encoding gag.

The nucleotide sequence encoding gag may be a truncated nucleotide sequence encoding a part of gag. The nucleotide sequence encoding gag may be a minimal truncated nucleotide sequence encoding a part of gag. The part of gag may be a contiguous sequence. The truncated nucleotide sequence or minimal truncated nucleotide sequence encoding a part of gag may also contain at least one frameshift mutation.

An example truncated nucleotide sequence encoding a part of gag and which contains a frameshift mutation at position 45-46 is as follows:

(SEQ ID NO: 6)
ATGGGTGCGAGAGCGTCAGTATTAAGCGGGGGAGA
ATTAGATCGCGATGGGAAAAAATTCGGTTAAGGCC
AGGGGGAAAGAAAAAATATAAATTAAAACATATAG
TATGGGCAAGCAGGGAGCTAGAACGATTCGCAGTT
AATCCTGGCCTGTTAGAAACATCAGAAGGCTGTAG
ACAAATACTGGGACAGCTACAACCATCCCTTCAGA
CAGGATCAGAAGAACTTAGATCATTATATAATACA
GTAGCAACCCTCTATTGTGTGCATCAAAGGATAGA
GATAAAAGACACCAAGGAAGCTTTAGACAAGATAG
AGGGAGAGCAAAACAAAAGTAAGA.

An example minimal truncated nucleotide sequence encoding a part of gag and which contains a frameshift mutation at position 45-46 is as follows:

(SEQ ID NO: 7)
ATGGGTGCGAGAGCGTCAGTATTAAGCGGGGGAGA
ATTAGATCGCGATGGGAAAAAATTCGGTTAAGGCC
AGGGGGAAAGA.

The nucleotide sequence encoding gag may, for example, comprise:
a) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 6; or
b) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 7.

The nucleotide sequence encoding gag may comprise a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 6.

The nucleotide sequence encoding gag may comprise a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 7.

The modified nucleotide sequence encoding gag may comprise:
a) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 6; or
b) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 7.

The modified nucleotide sequence encoding gag may comprise a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 6.

The modified nucleotide sequence encoding gag may comprise a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 7.

The modified nucleotide sequence encoding gag may comprise the sequence as set forth in SEQ ID NO: 6 or SEQ ID NO: 7, or a sequence having at least 80% identity (suitably at least 85%, at least 90%, at least 91%, at least 92%, at least 93%, at least 94%, at least 95%, at least 96%, at least 97%, at least 98%, at least 99%) thereto, wherein at least one ATG sequence selected from (a) to (c) is mutated:
a) ATG corresponding to positions 1-3 of SEQ ID NO: 6;
b) ATG corresponding to positions 47-49 of SEQ ID NO: 6; and/or
c) ATG corresponding to positions 107-109 of SEQ ID NO: 6.

The modified nucleotide sequence encoding gag may comprise the sequence as set forth in SEQ ID NO: 6 or SEQ ID NO: 7, or a sequence having at least 80% identity (suitably at least 85%, at least 90%, at least 91%, at least 92%, at least 93%, at least 94%, at least 95%, at least 96%, at least 97%, at least 98%, at least 99%) thereto, wherein ATG corresponding to positions 1-3 of SEQ ID NO: 6 is mutated.

The modified nucleotide sequence encoding gag may comprise the sequence as set forth in SEQ ID NO: 6 or SEQ ID NO: 7, or a sequence having at least 80% identity (suitably at least 85%, at least 90%, at least 91%, at least 92%, at least 93%, at least 94%, at least 95%, at least 96%, at least 97%, at least 98%, at least 99%) thereto, wherein ATG corresponding to positions 47-49 of SEQ ID NO: 6 is mutated.

The modified nucleotide sequence encoding gag may comprise the sequence as set forth in SEQ ID NO: 6 or SEQ ID NO: 7, or a sequence having at least 80% identity (suitably at least 85%, at least 90%, at least 91%, at least 92%, at least 93%, at least 94%, at least 95%, at least 96%, at least 97%, at least 98%, at least 99%) thereto, wherein ATG corresponding to positions 107-109 of SEQ ID NO: 6 is mutated.

An example modified truncated nucleotide sequence encoding part of gag and which contains a frameshift mutation is as follows:

(SEQ ID NO: 8)
ACGGGTGCGAGAGCGTCAGTATTAAGCGGGGGAGA
ATTAGATCGCGATTGGGAAAAAATTCGGTTAAGGC
CAGGGGGAAAGAAAAAATATAAATTAAAACATATA
GTATTGGGCAAGCAGGGAGCTAGAACGATTCGCAG
TTAATCCTGGCCTGTTAGAAACATCAGAAGGCTGT
AGACAAATACTGGGACAGCTACAACCATCCCTTCA
GACAGGATCAGAAGAACTTAGATCATTATATAATA
CAGTAGCAACCCTCTATTGTGTGCATCAAAGGATA
GAGATAAAAGACACCAAGGAAGCTTTAGACAAGAT
AGAGGGAGAGCAAAACAAAAGTAAGA.

An example modified minimal truncated nucleotide sequence encoding a part of gag and which contains a frameshift mutation is as follows:

(SEQ ID NO: 9)
ACGGGTGCGAGAGCGTCAGTATTAAGCGGGGGAGA
ATTAGATCGCGATTGGGAAAAAATTCGGTTAAGGC
CAGGGGGAAAGA.

The modified nucleotide sequence encoding gag may comprise the sequence as set forth in SEQ ID NO: 8, or a sequence having at least 80% (suitably at least 85%, at least 90%, at least 91%, at least 92%, at least 93%, at least 94%, at least 95%, at least 96%, at least 97%, at least 98%, at least 99%) identity thereto. The sequence may comprise less than three (suitably less than two or less than one) ATG sequences.

The modified nucleotide sequence encoding gag may comprise the sequence as set forth in SEQ ID NO: 9, or a sequence having at least 80% (suitably at least 85%, at least 90%, at least 91%, at least 92%, at least 93%, at least 94%, at least 95%, at least 96%, at least 97%, at least 98%, at least 99%) identity thereto. The sequence may comprise less than two (suitably less than one) ATG sequences.

The modified nucleotide sequence encoding gag may comprise less than three ATG sequences. Suitably, the modified nucleotide sequence encoding gag may comprise less than two or less than one ATG sequence(s). The modified nucleotide sequence encoding gag may lack an ATG sequence.

The lentiviral vector genome as described herein may lack a nucleotide sequence encoding Gag-p17 or a fragment thereof. The lentiviral vector genome may, for example, not express Gag-p17 or a fragment thereof. In one embodiment, the lentiviral vector genome may lack the sequence as set forth in SEQ ID NO: 10.

The lentiviral vector genome may be an RNA genome of a lentiviral vector.

The lentiviral vector as described herein may be a transgene expression cassette.

In one embodiment, the lentiviral vector is derived from HIV-1, HIV-2, SIV, FIV, BIV, EIAV, CAEV or Visna lentivirus.

In one embodiment, the lentiviral vector genome as described herein lacks ATG sequences in the backbone of the vector genome. In one embodiment, the lentiviral vector genome as described herein lacks ATG sequences except in the NOI (transgene).

In a further aspect, the present invention provides a nucleotide sequence encoding the lentiviral vector genome as described herein.

In a further aspect, the present invention provides a lentiviral vector genome comprising at least one modified viral cis-acting sequence, wherein at least one internal open reading frame (ORF) in the viral cis-acting sequence is ablated.

In a further aspect, the present invention provides a lentiviral vector genome comprising at least one modified viral cis-acting sequence, wherein at least one internal open reading frame (ORF) in the viral cis-acting sequence is silenced.

Modified Rev Response Element (RRE)

The present invention provides a lentiviral vector genome comprising a modified Rev response element (RRE), wherein at least one internal open reading frame (ORF) in the RRE is disrupted.

ORFs present in the vector backbone delivered in transduced (e.g. patient) cells could be transcribed, for example, when read-through transcription from upstream cellular promoters occurs (lentiviral vectors target active transcription sites), leading to potential aberrant transcription of genetic material located in the vector backbone in patient cells. This potential aberrant transcription of genetic material located in the vector backbone following read-through transcription could also occur during lentiviral vector production in production cells.

The RRE is an essential viral RNA element that is well conserved across lentiviral vectors and across different wild-type HIV-1 isolates. The RRE present within lentiviral vector genomes may contain multiple internal ORFs. These internal ORFs may be found between an internal ATG sequence of the RRE and the stop codon immediately 3′ to the ATG sequence.

The RRE present within lentiviral vector genomes is typically embedded within the packaging region containing highly structured RNA towards the 5′ region of the RNA (the 5′UTR). The 5′ UTR structure consists of series of stem-loop structures connected by small linkers. These stem-loops include the RRE. Thus, the RRE itself has a complex secondary structure, involving complementary base-pairing, to which Rev binds.

The present inventors have surprisingly found that modifications in the RRE to disrupt at least one internal ORF, for example by mutating the ATG sequence which denotes the start of the at least one internal ORF, are tolerated. Thus, the modified RREs described herein retain Rev binding capacity.

In some embodiments, at least two (suitably at least three, at least four, at least five, at least six, at least seven or at least eight) internal ORFs in the RRE may be disrupted. In some embodiments, at least three internal ORFs in the RRE may be disrupted.

In some embodiments, one (suitably, two, three, four, five, six, seven or eight) internal ORFs in the RRE may be disrupted.

In some embodiments, the at least one internal ORF may be disrupted such that the internal ORF is not expressed. In some embodiments, the at least one internal ORF may be disrupted such that the internal ORF is not translated. In some embodiments, the at least one internal ORF may be disrupted such that no protein is expressed from the internal ORF. In some embodiments, the at least one internal ORF may be disrupted such that no protein is translated from the internal ORF. Thus, the at least one internal ORF present in the modified RRE in the vector backbone delivered in transduced cells may be disrupted such that aberrant transcription of the internal ORF is prevented when there is read-through transcription from upstream cellular promoters.

In one embodiment, the at least one internal ORF may be disrupted by mutating at least one ATG sequence. A “mutation” of an ATG sequence may comprise one or more nucleotide deletions, additions, or substitutions.

In one embodiment, the at least one ATG sequence may be mutated in the modified RRE to a sequence selected from the group consisting of:
a) an ATTG sequence;
b) an ACG sequence;
c) an A-G sequence;
d) an AAG sequence;
e) a TTG sequence; and/or
f) an ATT sequence.

The at least one ATG sequence may be mutated to an ATTG sequence in the modified RRE. The at least one ATG sequence may be mutated to an ACG sequence in the modified RRE. The at least one ATG sequence may be mutated to an A-G sequence in the modified RRE. The at least one ATG sequence may be mutated to an AAG sequence in the modified RRE. The at least one ATG sequence may be mutated to a TTG sequence in the modified RRE. The at least one ATG sequence may be mutated to an ATT sequence in the modified RRE.

The modified RRE may comprise less than eight ATG sequences.

Accordingly, in a further aspect the present invention provides a lentiviral vector genome comprising a modified Rev response element (RRE), wherein the modified RRE comprises less than eight ATG sequences.

Suitably, the modified RRE may comprise less than seven, less than six, less than five, less than four, less than three, less than two or less than one ATG sequence(s). The modified RRE may lack an ATG sequence.

The RRE may be a minimal functional RRE. An example minimal functional RRE is as follows:

(SEQ ID NO: 1)
AGGAGCTTTGTTCCTTGGGTTCTTGGGAGCAGCAG
GAAGCACTATGGGCGCAGCGTCAATGACGCTGACG
GTACAGGCCAGACAATTATTGTCTGGTATAGTGCA
GCAGCAGAACAATTTGCTGAGGGCTATTGAGGCGC
AACAGCATCTGTTGCAACTCACAGTCTGGGGCATC
AAGCAGCTCCAGGCAAGAATCCTGGCTGTGGAAAG
ATACCTAAAGGATCAACAGCTCCT.

By “minimal functional RRE” or “minimal RRE” is meant a truncated RRE sequence which retains the function of the full-length RRE. Thus, the minimal functional RRE retains Rev binding capacity.

The RRE may be a full-length RRE. An example full-length RRE is as follows:

(SEQ ID NO: 2)
TGATCTTCAGACCTGGAGGAGGAGATATGAGGGAC
AATTGGAGAAGTGAATTATATAAATATAAAGTAGT
AAAAATTGAACCATTAGGAGTAGCACCCACCAAGG
CAAAGAGAAGAGTGGTGCAGAGAGAAAAAAGAGCA
GTGGGAATAGGAGCTTTGTTCCTTGGGTTCTTGGG
AGCAGCAGGAAGCACTATGGGCGCAGCGTCAATGA
CGCTGACGGTACAGGCCAGACAATTATTGTCTGGT
ATAGTGCAGCAGCAGAACAATTTGCTGAGGGCTAT
TGAGGCGCAACAGCATCTGTTGCAACTCACAGTCT
GGGGCATCAAGCAGCTCCAGGCAAGAATCCTGGCT
GTGGAAAGATACCTAAAGGATCAACAGCTCCTGGG
GATTTGGGGTTGCTCTGGAAAACTCATTTGCACCA
CTGCTGTGCCTTGGAATGCTAGTTGGAGTAATAAA
TCTCTGGAACAGATTTGGAATCACACGACCTGGAT
GGAGTGGGACAGAGAAATTAACAATTACACAAGCT
TAATACACTCCTTAATTGAAGAATCGCAAAACCAG
CAAGAAAAGAATGAACAAGAATTATTGGAATTAGA
TAAATGGGCAAGTTTGTGGAATTGGTTTAACATAA
CAAATTGGCTGTGGTATATAAAATTATTCATAATG
ATAGTAGGAGGCTTGGTAGGTTTAAGAATAGTTTT
TGCTGTACTTTCTATAGTGAATAGAGTTAGGCAGG
GATATTCACCATTATCGTTTCAGACCCACCTCCCA
ACCCCGAGGGGAC.

The RRE may comprise:
a) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 1; and/or
b) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 2.

The RRE may comprise a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 1.

The RRE may comprise a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 2.

The modified RRE may comprise:
a) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 1; and/or
b) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 2.

The modified RRE may comprise a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 1.

The modified RRE may comprise a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 2.

The modified RRE may comprise the sequence as set forth in SEQ ID NO: 1 or SEQ ID NO: 2, or a sequence having at least 80% identity (suitably at least 85%, at least 90%, at least 91%, at least 92%, at least 93%, at least 94%, at least 95%, at least 96%, at least 97%, at least 98%, at least 99%) thereto, wherein at least one ATG sequence selected from the group (a)-(h) is mutated:
a) ATG corresponding to positions 27-29 of SEQ ID NO: 2;
b) ATG corresponding to positions 192-194 of SEQ ID NO: 2;
c) ATG corresponding to positions 207-209 of SEQ ID NO: 2;
d) ATG corresponding to positions 436-438 of SEQ ID NO: 2;
e) ATG corresponding to positions 489-491 of SEQ ID NO: 2;
f) ATG corresponding to positions 571-573 of SEQ ID NO: 2;
g) ATG corresponding to positions 599-601 of SEQ ID NO: 2; and/or
h) ATG corresponding to positions 663-665 of SEQ ID NO: 2.

The modified RRE may comprise the sequence as set forth in SEQ ID NO: 1 or SEQ ID NO: 2, or a sequence having at least 80% (suitably at least 85%, at least 90%, at least 91%, at least 92%, at least 93%, at least 94%, at least 95%, at least 96%, at least 97%, at least 98%, at least 99%) identity thereto, wherein ATG corresponding to positions 27-29 of SEQ ID NO: 2 is mutated.

The modified RRE may comprise the sequence as set forth in SEQ ID NO: 1 or SEQ ID NO: 2, or a sequence having at least 80% identity (suitably at least 85%, at least 90%, at least 91%, at least 92%, at least 93%, at least 94%, at least 95%, at least 96%, at least 97%, at least 98%, at least 99%) thereto, wherein ATG corresponding to positions 192-194 of SEQ ID NO: 2 is mutated.

The modified RRE may comprise the sequence as set forth in SEQ ID NO: 1 or SEQ ID NO: 2, or a sequence having at least 80% (suitably at least 85%, at least 90%, at least 91%, at least 92%, at least 93%, at least 94%, at least 95%, at least 96%, at least 97%, at least 98%, at least 99%) identity thereto, wherein ATG corresponding to positions 207-209 of SEQ ID NO: 2 is mutated.

The modified RRE may comprise the sequence as set forth in SEQ ID NO: 1 or SEQ ID NO: 2, or a sequence having at least 80% (suitably at least 85%, at least 90%, at least 91%, at least 92%, at least 93%, at least 94%, at least 95%, at least 96%, at least 97%, at least 98%, at least 99%) identity thereto, wherein ATG corresponding to positions 436-438 of SEQ ID NO: 2 is mutated.

The modified RRE may comprise the sequence as set forth in SEQ ID NO: 1 or SEQ ID NO: 2, or a sequence having at least 80% (suitably at least 85%, at least 90%, at least 91%, at least 92%, at least 93%, at least 94%, at least 95%, at least 96%, at least 97%, at least 98%, at least 99%) identity thereto, wherein ATG corresponding to positions 489-491 of SEQ ID NO: 2 is mutated.

The modified RRE may comprise the sequence as set forth in SEQ ID NO: 1 or SEQ ID NO: 2, or a sequence having at least 80% (suitably at least 85%, at least 90%, at least 91%, at least 92%, at least 93%, at least 94%, at least 95%, at least 96%, at least 97%, at least 98%, at least 99%) identity thereto, wherein ATG corresponding to positions 571-573 of SEQ ID NO: 2 is mutated.

The modified RRE may comprise the sequence as set forth in SEQ ID NO: 1 or SEQ ID NO: 2, or a sequence having at least 80% (suitably at least 85%, at least 90%, at least 91%, at least 92%, at least 93%, at least 94%, at least 95%, at least 96%, at least 97%, at least 98%, at least 99%) identity thereto, wherein ATG corresponding to positions 599-601 of SEQ ID NO: 2 is mutated.

The modified RRE may comprise the sequence as set forth in SEQ ID NO: 1 or SEQ ID NO: 2, or a sequence having at least 80% (suitably at least 85%, at least 90%, at least 91%, at least 92%, at least 93%, at least 94%, at least 95%, at least 96%, at least 97%, at least 98%, at least 99%) identity thereto, wherein ATG corresponding to positions 663-665 of SEQ ID NO: 2 is mutated.

An example modified RRE sequence is as follows:

(SEQ ID NO: 3)
AGGAGCTTTGTTCCTTGGGTTCTTGGGAGCAGCAG
GAAGCACTATTGGGCGCAGCGTCAATTGACGCTGA
CGGTACAGGCCAGACAATTATTGTCTGGTATAGTG
CAGCAGCAGAACAATTTGCTGAGGGCTATTGAGGC
GCAACAGCATCTGTTGCAACTCACAGTCTGGGGCA
TCAAGCAGCTCCAGGCAAGAATCCTGGCTGTGGAA
AGATACCTAAAGGATCAACAGCTCCT.

A further example modified RRE sequence is as follows:

(SEQ ID NO: 4)
TGATCTTCAGACCTGGAGGAGGAGATATTGAGGGA
CAATTGGAGAAGTGAATTATATAAATATAAAGTAG
TAAAAATTGAACCATTAGGAGTAGCACCCACCAAG
GCAAAGAGAAGAGTGGTGCAGAGAGAAAAAAGAGC
AGTGGGAATAGGAGCTTTGTTCCTTGGGTTCTTGG
GAGCAGCAGGAAGCACTATTGGGCGCAGCGTCAAT
TGACGCTGACGGTACAGGCCAGACAATTATTGTCT
GGTATAGTGCAGCAGCAGAACAATTTGCTGAGGGC
TATTGAGGCGCAACAGCATCTGTTGCAACTCACAG
TCTGGGGCATCAAGCAGCTCCAGGCAAGAATCCTG
GCTGTGGAAAGATACCTAAAGGATCAACAGCTCCT
GGGGATTTGGGGTTGCTCTGGAAAACTCATTTGCA
CCACTGCTGTGCCTTGGAATTGCTAGTTGGAGTAA
TAAATCTCTGGAACAGATTTGGAATCACACGACCT
GGATTGGAGTGGGACAGAGAAATTAACAATTACAC
AAGCTTAATACACTCCTTAATTGAAGAATCGCAAA
ACCAGCAAGAAAAGAATGAACAAGAATTATTGGAA
TTAGATAAATGGGCAAGTTTGTGGAATTGGTTTAA
CATAACAAATTGGCTGTGGTATATAAAATTATTCA
TAATTGATAGTAGGAGGCTTGGTAGGTTTAAGAAT
AGTTTTTGCTGTACTTTCTATAGTGAATAGAGTTA
GGCAGGGATATTCACCATTATCGTTTCAGACCCAC
CTCCCAACCCCGAGGGGAC.

An example of a modified RRE sequence lacking an ATG sequence is as follows:

(SEQ ID NO: 5)
TGATCTTCAGACCTGGAGGAGGAGATATTGAGGGA
CAATTGGAGAAGTGAATTATATAAATATAAAGTAG
TAAAAATTGAACCATTAGGAGTAGCACCCACCAAG
GCAAAGAGAAGAGTGGTGCAGAGAGAAAAAAGAGC
AGTGGGAATAGGAGCTTTGTTCCTTGGGTTCTTGG
GAGCAGCAGGAAGCACTATTGGGCGCAGCGTCAAT
TGACGCTGACGGTACAGGCCAGACAATTATTGTCT
GGTATAGTGCAGCAGCAGAACAATTTGCTGAGGGC
TATTGAGGCGCAACAGCATCTGTTGCAACTCACAG
TCTGGGGCATCAAGCAGCTCCAGGCAAGAATCCTG
GCTGTGGAAAGATACCTAAAGGATCAACAGCTCCT
GGGGATTTGGGGTTGCTCTGGAAAACTCATTTGCA
CCACTGCTGTGCCTTGGAATTGCTAGTTGGAGTAA
TAAATCTCTGGAACAGATTTGGAATCACACGACCT
GGATTGGAGTGGGACAGAGAAATTAACAATTACAC
AAGCTTAATACACTCCTTAATTGAAGAATCGCAAA
ACCAGCAAGAAAAGAATtGAACAAGAATTATTGGA
ATTAGATAAATtGGGCAAGTTTGTGGAATTGGTTT
AACATAACAAAT
TGGCTGTGGTATATAAAATTATTCATAATTGATAG
TAGGAGGCTTGGTAGGTTTAAGAATAGTTTTTGCT
GTACTTTCTATAGTGAATAGAGTTAGGCAGGGATA
TTCACCATTATCGTTTCAGACCCACCTCCCAACCC
CGAGGGGAC.

The modified RRE may comprise the sequence as set forth in SEQ ID NO: 3 or SEQ ID NO: 4 or SEQ ID NO: 5, or a sequence having at least 80% (suitably at least 85%, at least 90%, at least 91%, at least 92%, at least 93%, at least 94%, at least 95%, at least 96%, at least 97%, at least 98%, at least 99%) identity thereto. The sequence may comprise less than eight (suitably less than seven, less than six, less than five, less than four, less than three, less than two or less than one) ATG sequences.

Woodchuck Hepatitis Virus (WHV) Post-Transcriptional Regulatory Element (WPRE)

In one aspect, the present invention provides a lentiviral vector genome comprising a modified Woodchuck hepatitis virus (WHV) post-transcriptional regulatory element (WPRE), wherein at least one internal open reading frame (ORF) in the WPRE is disrupted.

The WPRE can enhance expression from a number of different vector types including lentiviral vectors (U.S. Pat. Nos. 6,136,597; 6,287,814; Zufferey, R., et al. (1999) J. Virol. 73: 2886-92). Without wanting to be bound by theory, this enhancement is thought to be due to improved RNA processing at the post-transcriptional level, resulting in increased levels of nuclear transcripts. A two-fold increase in mRNA stability also contributes to this enhancement (Zufferey, R., et al. ibid). The level of enhancement of protein expression from transcripts containing the WPRE versus those without the WPRE has been reported to be around 2-to-5 fold, and correlates well with the increase in transcript levels. This has been demonstrated with a number of different transgenes (Zufferey, R., et al. ibid).

The WPRE contains three cis-acting sequences important for its function in enhancing expression levels. In addition, it contains a fragment of approximately 180 bp comprising the 5′-end of the WHV X protein ORF (full length ORF is 425 bp), together with its associated promoter. The full-length X protein has been implicated in tumorigenesis (Flajolet, M. et al, (1998) J. Virol. 72: 6175-6180). Translation from transcripts initiated from the X promoter results in formation of a protein representing the NH₂-terminal 60 amino acids of the X protein. This truncated X protein can promote tumorigenesis, particularly if the truncated X protein sequence is integrated into the host cell genome at specific loci (Balsano, C. et al, (1991) Biochem. Biophys Res. Commun. 176: 985-92; Flajolet, M. et al, (1998) J. Virol. 72: 6175-80; Zheng, Y. W., et al, (1994) J. Biol. Chem. 269: 22593-8; Runkel, L., et al, (1993) Virology 197: 529-36). Therefore, expression of the truncated X protein could promote tumorigenesis if delivered to cells of interest, precluding safe use of wild-type WPRE sequences.

US 2005/0002907 discloses that mutation of a region of the WPRE corresponding to the X protein ORF ablates the tumorigenic activity of the X protein, thereby allowing the WPRE to be used safely in retroviral and lentiviral expression vectors to enhance expression levels of heterologous genes or nucleotides of interest.

As used herein, the “X region” of the WPRE is defined as comprising at least the first 60-amino acids of the X protein ORF, including the translation initiation codon, and its associated promoter. A “functional” X protein is defined herein as a truncated X protein that is capable of promoting tumorigenesis, or a transformed phenotype, when expressed in cells of interest. A “non-functional” X protein in the context of this application is defined as an X protein that is incapable of promoting tumorigenesis in cells of interest.

The modified WPREs described herein retain the capacity to enhance expression from the lentiviral vector.

In some embodiments, at least two (suitably at least three, at least four, at least five, at least six or at least seven) internal ORFs in the WPRE may be disrupted. In some embodiments, at least three internal ORFs in the WPRE may be disrupted.

In some embodiments, one (suitably, two, three, four, five, six or seven) internal ORFs in the WPRE may be disrupted.

In some embodiments, the at least one internal ORF may be disrupted such that the internal ORF is not expressed. In some embodiments, the at least one internal ORF may be disrupted such that the internal ORF is not translated. In some embodiments, the at least one internal ORF may be disrupted such that no protein is expressed from the internal ORF. In some embodiments, the at least one internal ORF may be disrupted such that no protein is translated from the internal ORF. Thus, the at least one internal ORF present in the modified WPRE in the vector backbone delivered in transduced cells may be disrupted such that aberrant transcription of the internal ORF is prevented when there is read-through transcription from upstream cellular promoters.

In one embodiment, the at least one internal ORF may be disrupted by mutating at least one ATG sequence. A “mutation” of an ATG sequence may comprise one or more nucleotide deletions, additions, or substitutions.

In one embodiment, the at least one ATG sequence may be mutated in the modified WPRE to a sequence selected from the group consisting of:
a) an ATTG sequence;
b) an ACG sequence;
c) an A-G sequence;
d) an AAG sequence;
e) a TTG sequence; and/or
f) an ATT sequence.

The at least one ATG sequence may be mutated to an ATTG sequence in the modified WPRE. The at least one ATG sequence may be mutated to an ACG sequence in the modified WPRE. The at least one ATG sequence may be mutated to an A-G sequence in the modified WPRE. The at least one ATG sequence may be mutated to an AAG sequence in the modified WPRE. The at least one ATG sequence may be mutated to a TTG sequence in the modified WPRE. The at least one ATG sequence may be mutated to an ATT sequence in the modified WPRE.

The modified WPRE may comprise less than seven ATG sequences. The modified WPRE may comprise less than six ATG sequences.

Accordingly, in a further aspect the present invention provides a lentiviral vector genome comprising a modified Woodchuck hepatitis virus (WHV) post-transcriptional regulatory element (WPRE), wherein the modified WPRE comprises less than seven ATG sequences.

In a further aspect the present invention provides a lentiviral vector genome comprising a modified Woodchuck hepatitis virus (WHV) post-transcriptional regulatory element (WPRE), wherein the modified WPRE comprises less than six ATG sequences.

Suitably, the modified WPRE may comprise less than seven, less than six, less than five, less than four, less than three, less than two or less than one ATG sequence(s). The modified WPRE may lack ATG sequences.

In some embodiments, at least one ATG sequence in the X region of the WPRE is mutated, whereby expression of a functional X protein is prevented. In preferred embodiments, the mutation is in the translation initiation codon of the X region. As a result of the mutation of the at least one ATG sequence, the X protein may not be expressed.

In some embodiments, the modified WPRE does not comprise a mutation in an ATG sequence in the X region of the WPRE.

An example WPRE sequence is as follows:

(SEQ ID NO: 11)
AATCAACCTCTGGATTACAAAATTTGTGAAAGATT
GACTGGTATTCTTAACTATGTTGCTCCTTTTACGC
TATGTGGATACGCTGCTTTAATGCCTTTGTATCAT
GCTATTGCTTCCCGTATGGCTTTCATTTTCTCCTC
CTTGTATAAATCCTGGTTGCTGTCTCTTTATGAGG
AGTTGTGGCCCGTTGTCAGGCAACGTGGCGTGGTG
TGCACTGTGTTTGCTGACGCAACCCCCACTGGTTG
GGGCATTGCCACCACCTGTCAGCTCCTTTCCGGGA
CTTTCGCTTTCCCCCTCCCTATTGCCACGGCGGAA
CTCATCGCCGCCTGCCTTGCCCGCTGCTGGACAGG
GGCTCGGCTGTTGGGCACTGACAATTCCGTGGTGT
TGTCGGGGAAGCTGACGTCCTTTCCATGGCTGCTC
GCCTGTGTTGCCACCTGGATTCTGCGCGGGACGTC
CTTCTGCTACGTCCCTTCGGCCCTCAATCCAGCGG
ACCTTCCTTCCCGCGGCCTGCTGCCGGCTCTGCGG
CCTCTTCCGCGTCTTCGCCTTCGCCCTCAGACGAG
TCGGATCTCCCTTTGGGCCGCCTCC.

An example WPRE sequence which contains a disrupted X-protein ORF is as follows:

(SEQ ID NO: 12)
AATCAACCTCTGGATTACAAAATTTGTGAAAGATT
GACTGGTATTCTTAACTATGTTGCTCCTTTTACGC
TATGTGGATACGCTGCTTTAATGCCTTTGTATCAT
GCTATTGCTTCCCGTATGGCTTTCATTTTCTCCTC
CTTGTATAAATCCTGGTTGCTGTCTCTTTATGAGG
AGTTGTGGCCCGTTGTCAGGCAACGTGGCGTGGTG
TGCACTGTGTTTGCTGACGCAACCCCCACTGGTTG
GGGCATTGCCACCACCTGTCAGCTCCTTTCCGGGA
CTTTCGCTTTCCCCCTCCCTATTGCCACGGCGGAA
CTCATCGCCGCCTGCCTTGCCCGCTGCTGGACAGG
GGCTCGGCTGTTGGGCACTGACAATTCCGTGGTGT
TGTCGGGGAAATCATCGTCCTTTCCTTGGCTGCTC
GCCTGTGTTGCCACCTGGATTCTGCGCGGGACGTC
CTTCTGCTACGTCCCTTCGGCCCTCAATCCAGCGG
ACCTTCCTTCCCGCGGCCTGCTGCCGGCTCTGCGG
CCTCTTCCGCGTCTTCGCCTTCGCCCTCAGACGAG
TCGGATCTCCCTTTGGGCCGCCTCC.

The WPRE may comprise:
a) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 11; and/or
b) a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 12.

The WPRE may comprise a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 11.

The WPRE may comprise a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 12.

The modified WPRE may comprise a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 11.

The modified WPRE may comprise a sequence having at least 80% (suitably at least 85%, at least 90%, at least 91%, at least 92%, at least 93%, at least 94%, at least 95%, at least 96%, at least 97%, at least 98%, at least 99%) identity to SEQ ID NO: 12.

The modified WPRE may comprise the sequence as set forth in SEQ ID NO: 11 or SEQ ID NO: 12, or a sequence having at least 80% identity (suitably at least 85%, at least 90%, at least 91%, at least 92%, at least 93%, at least 94%, at least 95%, at least 96%, at least 97%, at least 98%, at least 99%) thereto, wherein at least one ATG sequence selected from the group (a)-(g) is mutated:
a) ATG corresponding to positions 53-55 of SEQ ID NO: 11;
b) ATG corresponding to positions 72-74 of SEQ ID NO: 11;
c) ATG corresponding to positions 91-93 of SEQ ID NO: 11;
d) ATG corresponding to positions 104-106 of SEQ ID NO: 11;
e) ATG corresponding to positions 121-123 of SEQ ID NO: 11;
f) ATG corresponding to positions 170-172 of SEQ ID NO: 11; and/or
g) ATG corresponding to positions 411-413 of SEQ ID NO: 11.

The modified WPRE may comprise the sequence as set forth in SEQ ID NO: 11 or SEQ ID NO: 12, or a sequence having at least 80% identity (suitably at least 85%, at least 90%, at least 91%, at least 92%, at least 93%, at least 94%, at least 95%, at least 96%, at least 97%, at least 98%, at least 99%) thereto, wherein ATG corresponding to positions 53-55 of SEQ ID NO: 11 is mutated.

The modified WPRE may comprise the sequence as set forth in SEQ ID NO: 11 or SEQ ID NO: 12, or a sequence having at least 80% identity (suitably at least 85%, at least 90%, at least 91%, at least 92%, at least 93%, at least 94%, at least 95%, at least 96%, at least 97%, at least 98%, at least 99%) thereto, wherein ATG corresponding to positions 72-74 of SEQ ID NO: 11 is mutated.

The modified WPRE may comprise the sequence as set forth in SEQ ID NO: 11 or SEQ ID NO: 12, or a sequence having at least 80% identity (suitably at least 85%, at least 90%, at least 91%, at least 92%, at least 93%, at least 94%, at least 95%, at least 96%, at least 97%, at least 98%, at least 99%) thereto, wherein ATG corresponding to positions 91-93 of SEQ ID NO: 11 is mutated.

The modified WPRE may comprise the sequence as set forth in SEQ ID NO: 11 or SEQ ID NO: 12, or a sequence having at least 80% identity (suitably at least 85%, at least 90%, at least 91%, at least 92%, at least 93%, at least 94%, at least 95%, at least 96%, at least 97%, at least 98%, at least 99%) thereto, wherein ATG corresponding to positions 104-106 of SEQ ID NO: 11 is mutated;

The modified WPRE may comprise the sequence as set forth in SEQ ID NO: 11 or SEQ ID NO: 12, or a sequence having at least 80% identity (suitably at least 85%, at least 90%, at least 91%, at least 92%, at least 93%, at least 94%, at least 95%, at least 96%, at least 97%, at least 98%, at least 99%) thereto, wherein ATG corresponding to positions 121-123 of SEQ ID NO: 11 is mutated;

The modified WPRE may comprise the sequence as set forth in SEQ ID NO: 11 or SEQ ID NO: 12, or a sequence having at least 80% identity (suitably at least 85%, at least 90%, at least 91%, at least 92%, at least 93%, at least 94%, at least 95%, at least 96%, at least 97%, at least 98%, at least 99%) thereto, wherein ATG corresponding to positions 170-172 of SEQ ID NO: 11 is mutated; and/or

The modified WPRE may comprise the sequence as set forth in SEQ ID NO: 11 or SEQ ID NO: 12, or a sequence having at least 80% identity (suitably at least 85%, at least 90%, at least 91%, at least 92%, at least 93%, at least 94%, at least 95%, at least 96%, at least 97%, at least 98%, at least 99%) thereto, wherein ATG corresponding to positions 411-413 of SEQ ID NO: 11 is mutated.

The WRPE typically contains a retained Pol ORF. An example retained Pol ORF sequence is as follows:

(SEQ ID NO: 13)
ATGCTATTGCTTCCCGTATGGCTTTCATTTTCTCC
TCCTTGTATAAATCCTGGTTGCTGTCTCTTTATGA
GGAGTTGTGGCCCGTTGTCAGGCAACGTGGCGTGG
TGTGCACTGTGTTTGCTGACGCAACCCCCACTGGT
TGGGGCATTGCCACCACCTGTCAGCTCCTTTCCGG
GACTTTCGCTTTCCCCCTCCCTATTGCCACGGCGG
AACTCATCGCCGCCTGCCTTGCCCGCTGCTGGACA
GGGGCTCGGCTGTTGGGCACTGACAATTCCGTGGT
GTTGTCGGGGAAATCATCGTCCTTTCCTTGGCTGC
TCGCCTGTGTTGCCACCTGGATTCTGCGCGGGACG
TCCTTCTGCTACGTCCCTTCGGCCCTCAATCCAGC
GGACCTTCCTTCCCGCGGCCTGCTGCCGGCTCTGC
GGCCTCTTCCGCGTCTTCGCCTTCGCCCTCAGACG
AGTCGGATCTCCCTTTGGGCCGCCTCC.

In one embodiment, at least one (suitably at least two or at least three) ATG sequence within the retained Pol ORF sequence in the WPRE is mutated. In one embodiment, all ATG sequences within the retained Pol ORF sequence in the WPRE are mutated.

In one embodiment, the modified WPRE comprises less than three (suitably less than two or less than one) ATG sequences in the retained Pol ORF sequence in the WPRE. In one embodiment, the modified WPRE lacks an ATG sequence in the retained Pol ORF sequence in the WPRE.

An example modified WPRE sequence in which all ATG codons within the retained Pol ORF are mutated is as follows:

(SEQ ID NO: 14)
AATCAACCTCTGGATTACAAAATTTGTGAAAGATT
GACTGGTATTCTTAACTATGTTGCTCCTTTTACGC
TATGTGGATACGCTGCTTTAATGCCTTTGTATCAT
TGCTATTGCTTCCCGTATGGCTTTCATTTTCTCCT
CCTTGTATAAATCCTGGTTGCTGTCTCTTTATTGA
GGAGTTGTGGCCCGTTGTCAGGCAACGTGGCGTGG
TGTGCACTGTGTTTGCTGACGCAACCCCCACTGGT
TGGGGCATTGCCACCACCTGTCAGCTCCTTTCCGG
GACTTTCGCTTTCCCCCTCCCTATTGCCACGGCGG
AACTCATCGCCGCCTGCCTTGCCCGCTGCTGGACA
GGGGCTCGGCTGTTGGGCACTGACAATTCCGTGGT
GTTGTCGGGGAAATCATCGTCCTTTCCTTGGCTGC
TCGCCTGTGTTGCCACCTGGATTCTGCGCGGGACG
TCCTTCTGCTACGTCCCTTCGGCCCTCAATCCAGC
GGACCTTCCTTCCCGCGGCCTGCTGCCGGCTCTGC
GGCCTCTTCCGCGTCTTCGCCTTCGCCCTCAGACG
AGTCGGATCTCCCTTTGGGCCGCCTCC.

An example of a modified WPRE sequence lacking an ATG sequence is as follows:

(SEQ ID NO: 15)
AATCAACCTCTGGATTACAAAATTTGTGAAAGATT
GACTGGTATTCTTAACTATtGTTGCTCCTTTTACG
CTATtGTGGATACGCTGCTTTAATtGCCTTTGTAT
CATtGCTATTGCTTCCCGTATtGGCTTTCATTTTC
TCCTCCTTGTATAAATCCTGGTTGCTGTCTCTTTA
TtGAGGAGTTGTGGCCCGTTGTCAGGCAACGTGGC
GT
GGTGTGCACTGTGTTTGCTGACGCAACCCCCACTG
GTTGGGGCATTGCCACCACCTGTCAGCTCCTTTCC
GGGACTTTCGCTTTCCCCCTCCCTATTGCCACGGC
GGAACTCATCGCCGCCTGCCTTGCCCGCTGCTGGA
CAGGGGCTCGGCTGTTGGGCACTGACAATTCCGTG
GTGTTGTCGGGGAAATCATCGTCCTTTCCTTGGCT
GCTCGCCTGTGTTGCCACCTGGATTCTGCGCGGGA
CGTCCTTCTGCTACGTCCCTTCGGCCCTCAATCCA
GCGGACCTTCCTTCCCGCGGCCTGCTGCCGGCTCT
GCGGCCTCTTCCGCGTCTTCGCCTTCGCCCTCAGA
CGAGTCGGATCTCCCTTTGGGCCGCCTCC.

The modified WPRE may comprise the sequence as set forth in SEQ ID NO: 14 or SEQ ID NO: 15, or a sequence having at least 80% (suitably at least 85%, at least 90%, at least 91%, at least 92%, at least 93%, at least 94%, at least 95%, at least 96%, at least 97%, at least 98%, at least 99%) identity thereto. The sequence may comprise less than six (suitably less than five, less than four, less than three, less than two or less than one) ATG sequences.

Viral Gag-p17 Protein

The present invention provides a lentiviral vector genome lacking either (i) a nucleotide sequence encoding Gag-p17 or (ii) a fragment of a nucleotide sequence encoding Gag-p17.

The viral protein Gag-p17 surrounds the capsid of the lentiviral vector particle, and is in turn surrounded by the envelope protein. A nucleotide sequence encoding Gag-p17 has historically been included in lentiviral vector genomes for the production of therapeutic lentiviral vectors. The nucleotide sequence encoding Gag-p17 present within lentiviral vector genomes is typically embedded within the packaging region containing highly structured RNA towards the 5′ region of the RNA (the 5′UTR). The nucleotide sequence encoding Gag-p17 typically comprises an RNA instability sequence (INS), herein referred to as p17-INS.

The present inventors have surprisingly found that the deletion of p17-INS from the backbone of the lentiviral vector genome does not significantly impact vector titres during lentiviral vector production.

The lentiviral vector genome lacking a nucleotide sequence encoding Gag-p17 or p17-INS is of a smaller size compared to a lentiviral vector genome comprising a nucleotide sequence encoding Gag-p17 or p17-INS. Thus, the amount of viral DNA contained within the viral vector backbone delivered in transduced cells is reduced when a lentiviral vector genome lacking a nucleotide sequence encoding Gag-p17 or p17-INS is used. Further, the lentiviral vector genome lacking a nucleotide sequence encoding Gag-p17 or p17-INS may be used to deliver a transgene of larger size than the transgenes which can be delivered using a lentiviral vector genome containing a nucleotide sequence encoding Gag-p17 or p17-INS. Therefore, there are several reasons why it may be desirable to delete nucleotide sequence encoding Gag-p17 or p17-INS within the vector backbone.

The present invention provides a lentiviral vector genome lacking a nucleotide sequence encoding p17-INS or a fragment thereof.

An example p17-INS is as follows:

(SEQ ID NO: 10)
AAAAATATAAATTAAAACATATAGTATGGGCAAGC
AGGGAGCTAGAACGATTCGCAGTTAATCCTGGCCT
GTTAGAAACATCAGAAGGCTGTAGACAAATACTGG
GACAGCTACAACCATCCCTTCAGACAGGATCAGAA
GAACTTAGATCATTATATAATACAGTAGCAACCCT
CTATTGTGTGCATCAAAGGATAGAGATAAAAGACA
CCAAGGAAGCTTTAGACAAGATAGAGGGAGAGCAA
AACAAAAGTA.

In one embodiment, the lentiviral vector genome may lack the sequence as set forth in SEQ ID NO: 10.

In one embodiment, the fragment of a nucleotide sequence encoding Gag-p17 is a part of a full-length nucleotide sequence encoding Gag-p17. In one embodiment, the fragment comprises or consists of at least about 10 nucleotides (suitably at least about 20, at least about 30, at least about 40, at least about 50, at least about 100, at least about 150, at least about 200, at least about 250, at least about 300, at least about 350 nucleotides).

In one embodiment, the fragment of a nucleotide sequence encoding Gag-p17 may have a length which is between 1% and 99% of full-length nucleotide sequence encoding Gag-p17. Suitably, the fragment of a nucleotide sequence encoding Gag-p17 may have a length which is at least about 10% (suitably at least about 20%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%) of a full-length nucleotide sequence encoding Gag-p17, such as a native nucleotide sequence encoding Gag-p17. The fragment may be a contiguous region of a full-length nucleotide sequence encoding Gag-p17, such as a native nucleotide sequence encoding Gag-p17.

In one embodiment, the fragment of a nucleotide sequence encoding Gag-p17 may have a length which is between 1% and 99% of full-length nucleotide sequence encoding p17-INS. Suitably, the fragment of a nucleotide sequence encoding Gag-p17 may have a length which is at least about 10% (suitably at least about 20%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%) of a full-length nucleotide sequence encoding p17-INS, such as a native nucleotide sequence encoding p17-INS (e.g. SEQ ID NO: 10). The fragment may be a contiguous region of a full-length nucleotide sequence encoding p17-INS, such as a native nucleotide sequence encoding p17-INS (e.g. SEQ ID NO: 10).

In one embodiment, the fragment of a nucleotide sequence encoding Gag-p17 comprises or consists of the INS located in the nucleotide sequence encoding Gag-p17.

In one embodiment, the lentiviral vector genome lacking either (i) a nucleotide sequence encoding Gag-p17 or (ii) a fragment of a nucleotide sequence encoding Gag-p17 may comprise at least one modified viral cis-acting sequence as described herein.

In one embodiment, the lentiviral vector genome lacking either (i) a nucleotide sequence encoding Gag-p17 or (ii) a fragment of a nucleotide sequence encoding Gag-p17 may comprise a modified RRE as described herein.

In one embodiment, the lentiviral vector genome lacking either (i) a nucleotide sequence encoding Gag-p17 or (ii) a fragment of a nucleotide sequence encoding Gag-p17 may comprise a modified WPRE as described herein.

In one embodiment, the lentiviral vector genome lacking either (i) a nucleotide sequence encoding Gag-p17 or (ii) a fragment of a nucleotide sequence encoding Gag-p17 may comprise a modified RRE as described herein and a modified WPRE as described herein.

In one embodiment, the lentiviral vector genome lacking either (i) a nucleotide sequence encoding Gag-p17 or (ii) a fragment of a nucleotide sequence encoding Gag-p17 may comprise a modified nucleotide sequence encoding gag as described herein.

In one embodiment, the lentiviral vector genome lacking either (i) a nucleotide sequence encoding Gag-p17 or (ii) a fragment of a nucleotide sequence encoding Gag-p17 may comprise a modified RRE as described herein and a modified nucleotide sequence encoding gag as described herein.

In one embodiment, the lentiviral vector genome lacking either (i) a nucleotide sequence encoding Gag-p17 or (ii) a fragment of a nucleotide sequence encoding Gag-p17 may comprise a modified WPRE as described herein and a modified nucleotide sequence encoding gag as described herein.

In one embodiment, the lentiviral vector genome lacking either (i) a nucleotide sequence encoding Gag-p17 or (ii) a fragment of a nucleotide sequence encoding Gag-p17 may comprise a modified RRE as described herein, a modified WPRE as described herein and a modified nucleotide sequence encoding gag as described herein.

The lentiviral vector genome may be an RNA genome of a lentiviral vector.

In one embodiment, the lentiviral vector is derived from HIV-1, HIV-2, SIV, FIV, BIV, EIAV, CAEV or Visna lentivirus.

In a further aspect, the present invention provides a nucleotide sequence encoding the lentiviral vector genome as described herein.

Vector/Expression Cassette

A vector is a tool that allows or facilitates the transfer of an entity from one environment to another. In accordance with the present invention, and by way of example, some vectors used in recombinant nucleic acid techniques allow entities, such as a segment of nucleic acid (e.g. a heterologous DNA segment, such as a heterologous cDNA segment), to be transferred into and expressed by a target cell. The vector may facilitate the integration of the nucleotide sequence encoding a viral vector component to maintain the nucleotide sequence encoding the viral vector component and its expression within the target cell.

The vector may be or may include an expression cassette (also termed an expression construct). Expression cassettes as described herein comprise regions of nucleic acid containing sequences capable of being transcribed. Thus, sequences encoding mRNA, tRNA and rRNA are included within this definition.

The vector may contain one or more selectable marker genes (e.g. a neomycin resistance gene) and/or traceable marker gene(s) (e.g. a gene encoding green fluorescent protein (GFP)). Vectors may be used, for example, to infect and/or transduce a target cell. The vector may further comprise a nucleotide sequence enabling the vector to replicate in the host cell in question, such as a conditionally replicating oncolytic vector.

The term “cassette”—which is synonymous with terms such as “conjugate”, “construct” and “hybrid”—includes a polynucleotide sequence directly or indirectly attached to a promoter. The expression cassettes for use in the invention comprise a promoter for the expression of the nucleotide sequence encoding a viral vector component and optionally a regulator of the nucleotide sequence encoding the viral vector component. Preferably the cassette comprises at least a polynucleotide sequence operably linked to a promoter.

The choice of expression cassette, e.g. plasmid, cosmid, virus or phage vector, will often depend on the host cell into which it is to be introduced. The expression cassette can be a DNA plasmid (supercoiled, nicked or linearised), minicircle DNA (linear or supercoiled), plasmid DNA containing just the regions of interest by removal of the plasmid backbone by restriction enzyme digestion and purification, DNA generated using an enzymatic DNA amplification platform e.g. doggybone DNA (dbDNA™) where the final DNA used is in a closed ligated form or where it has been prepared (e.g. restriction enzyme digestion) to have open cut ends.

In a further aspect, the present invention provides a lentiviral vector comprising the lentiviral vector genome as described herein. The lentiviral vector may be derived from HIV-1, HIV-2, SIV, FIV, BIV, EIAV, CAEV or Visna lentivirus. The lentiviral vector may be in the form of a lentiviral vector particle.

In a further aspect, the present invention provides an expression cassette comprising the nucleotide sequence of the invention.

Lentiviral Vector Production Systems and Cells

A lentiviral vector production system comprises a set of nucleotide sequences encoding the components required for production of the lentiviral vector. Accordingly, a vector production system comprises a set of nucleotide sequences which encode the viral vector components necessary to generate lentiviral vector particles.

“Viral vector production system” or “vector production system” or “production system” is to be understood as a system comprising the necessary components for lentiviral vector production.

In one embodiment, the viral vector production system comprises nucleotide sequences encoding the lentiviral vector genome as described herein, Gag and Gag/Pol proteins, and Env protein. The production system may optionally comprise a nucleotide sequence encoding the Rev protein, or functional substitute thereof.

In one embodiment, the viral vector production system comprises modular nucleic acid constructs (modular constructs). A modular construct is a DNA expression construct comprising two or more nucleic acids used in the production of lentiviral vectors. A modular construct can be a DNA plasmid comprising two or more nucleic acids used in the production of lentiviral vectors. The plasmid may be a bacterial plasmid. The nucleic acids can encode for example, gag-pol, rev, env, vector genome. In addition, modular constructs designed for generation of packaging and producer cell lines may additionally need to encode transcriptional regulatory proteins (e.g. TetR, CymR) and/or translational repression proteins (e.g. TRAP) and selectable markers (e.g. Zeocin™, hygromycin, blasticidin, puromycin, neomycin resistance genes). Suitable modular constructs for use in the present invention are described in EP 3502260, which is hereby incorporated by reference in its entirety.

As the modular constructs for use in accordance with the present invention contain nucleic acid sequences encoding two or more of the retroviral components on one construct, the safety profile of these modular constructs has been considered and additional safety features directly engineered into the constructs. These features include the use of insulators for multiple open reading frames of retroviral vector components and/or the specific orientation and arrangement of the retroviral genes in the modular constructs. It is believed that by using these features the direct read-through to generate replication-competent viral particles will be prevented.

The nucleic acid sequences encoding the viral vector components may be in reverse and/or alternating transcriptional orientations in the modular construct. Thus, the nucleic acid sequences encoding the viral vector components are not presented in the same 5′ to 3′ orientation, such that the viral vector components cannot be produced from the same mRNA molecule. The reverse orientation may mean that at least two coding sequences for different vector components are presented in the ‘head-to-head’ and ‘tail-to-tail’ transcriptional orientations. This may be achieved by providing the coding sequence for one vector component, e.g. env, on one strand and the coding sequence for another vector component, e.g. rev, on the opposing strand of the modular construct. Preferably, when coding sequences for more than two vector components are present in the modular construct, at least two of the coding sequences are present in the reverse transcriptional orientation. Accordingly, when coding sequences for more than two vector components are present in the modular construct, each component may be orientated such that it is present in the opposite 5′ to 3′ orientation to all of the adjacent coding sequence(s) for other vector components to which it is adjacent, i.e. alternating 5′ to 3′ (or transcriptional) orientations for each coding sequence may be employed.

The modular construct for use according to the present invention may comprise nucleic acid sequences encoding two or more of the following vector components: the lentiviral vector genome as described herein, gag-pol, rev, env. The modular construct may comprise nucleic acid sequences encoding any combination of the vector components. In one embodiment, the modular construct may comprise nucleic acid sequences encoding:
i) the RNA genome of the retroviral vector and rev, or a functional substitute thereof;
ii) the RNA genome of the retroviral vector and gag-pol;
iii) the RNA genome of the retroviral vector and env;
iv) gag-pol and rev, or a functional substitute thereof;
v) gag-pol and env;
vi) env and rev, or a functional substitute thereof;
vii) the RNA genome of the retroviral vector, rev, or a functional substitute thereof, and gag-pol;
viii) the RNA genome of the retroviral vector, rev, or a functional substitute thereof, and env;
ix) the RNA genome of the retroviral vector, gag-pol and env; or
x) gag-pol, rev, or a functional substitute thereof, and env, wherein the nucleic acid sequences are in reverse and/or alternating orientations.

In one embodiment, a cell for producing retroviral vectors may comprise nucleic acid sequences encoding any one of the combinations i) to x) above, wherein the nucleic acid sequences are located at the same genetic locus and are in reverse and/or alternating orientations. The same genetic locus may refer to a single extrachromosomal locus in the cell, e.g. a single plasmid, or a single locus (i.e. a single insertion site) in the genome of the cell. The cell may be a stable or transient cell for producing retroviral vectors, e.g. lentiviral vectors. In one aspect the cell does not comprise tat.

The DNA expression construct can be a DNA plasmid (supercoiled, nicked or linearised), minicircle DNA (linear or supercoiled), plasmid DNA containing just the regions of interest by removal of the plasmid backbone by restriction enzyme digestion and purification, DNA generated using an enzymatic DNA amplification platform e.g. doggybone DNA (dbDNA™) where the final DNA used is in a closed ligated form or where it has been prepared (e.g. restriction enzyme digestion) to have open cut ends.

In a further aspect, the present invention provides a cell comprising the lentiviral vector genome of the invention, the nucleotide sequence of the invention, the expression cassette of the invention or the viral vector production system of the invention.

In a further aspect, the present invention provides a cell for producing lentiviral vectors comprising:
a) nucleotide sequences encoding vector components including gag-pol and env, and optionally rev, and a nucleotide sequence of the invention or the expression cassette of the invention; or
b) the viral vector production system of the invention; and
c) optionally a nucleotide sequence encoding a modified U1 snRNA and/or optionally a nucleotide sequence encoding TRAP.

In a further aspect, the present invention provides a method for producing a lentiviral vector, comprising the steps of:

(i) introducing:
a) nucleotide sequences encoding vector components including gag-pol and env, and optionally rev, and a nucleotide sequence of the invention or the expression cassette of the invention; or
b) the viral vector production system of the invention; and
c) optionally a nucleotide sequence encoding a modified U1 snRNA and/or optionally a nucleotide sequence encoding TRAP
   into a cell;
   (ii) optionally selecting for a cell which comprises the nucleotide sequences encoding vector components and the lentiviral vector genome; and
   (iii) culturing the cell under conditions suitable for the production of the lentiviral vector.

In a further aspect, the present invention provides a lentiviral vector produced by the method of the invention. The lentiviral vector comprises a lentiviral vector genome as described herein.

In a further aspect, the present invention provides the use of the lentiviral vector genome of the invention, the nucleotide sequence of the invention, the expression cassette of the invention, the viral vector production system of the invention, or the cell of the invention for producing a lentiviral vector.

A “viral vector production cell”, “vector production cell”, or “production cell” is to be understood as a cell that is capable of producing a lentiviral vector or lentiviral vector particle. Lentiviral vector production cells may be “producer cells” or “packaging cells”. One or more DNA constructs of the viral vector system may be either stably integrated or episomally maintained within the viral vector production cell. Alternatively, all the DNA components of the viral vector system may be transiently transfected into the viral vector production cell. In yet another alternative, a production cell stably expressing some of the components may be transiently transfected with the remaining components required for vector production.

As used herein, the term “packaging cell” refers to a cell which contains the elements necessary for production of lentiviral vector particles but which lacks the vector genome. Optionally, such packaging cells contain one or more expression cassettes which are capable of expressing viral structural proteins (such as gag, gag/pol and env).

Producer cells/packaging cells can be of any suitable cell type. Producer cells are generally mammalian cells but can be, for example, insect cells.

As used herein, the term “producer/production cell” or “vector producing/production cell” refers to a cell which contains all the elements necessary for production of lentiviral vector particles. The producer cell may be either a stable producer cell line or derived transiently or may be a stable packaging cell wherein the retroviral genome is transiently expressed.

In the methods of the invention, the vector components may include gag, env, rev and/or the RNA genome of the lentiviral vector when the viral vector is a lentiviral vector. The nucleotide sequences encoding vector components may be introduced into the cell either simultaneously or sequentially in any order.

The vector production cells may be cells cultured in vitro such as a tissue culture cell line. In some embodiments of the methods and uses of the invention, suitable production cells or cells for producing a lentiviral vector are those cells which are capable of producing viral vectors or viral vector particles when cultured under appropriate conditions. Thus, the cells typically comprise nucleotide sequences encoding vector components, which may include the RNA genome of the lentiviral vector as described herein, gag, env, and rev. Suitable cell lines include, but are not limited to, mammalian cells such as murine fibroblast derived cell lines or human cell lines. They are generally mammalian, including human cells, for example HEK293T, HEK293, CAP, CAP-T or CHO cells, but can be, for example, insect cells such as SF9 cells. Preferably, the vector production cells are derived from a human cell line. Accordingly, such suitable production cells may be employed in any of the methods or uses of the present invention.

Methods for introducing nucleotide sequences into cells are well known in the art and have been described previously. Thus, the introduction into a cell of nucleotide sequences encoding vector components including gag, env, rev and the RNA genome of the lentiviral vector, using conventional techniques in molecular and cell biology is within the capabilities of a person skilled in the art.

Stable production cells may be packaging or producer cells. To generate producer cells from packaging cells the vector genome DNA construct may be introduced stably or transiently. Packaging/producer cells can be generated by transducing a suitable cell line with a retroviral vector which expresses one of the components of the vector, i.e. a genome, the gag-pol components and an envelope as described in WO 2004/022761.

Alternatively, the nucleotide sequence can be transfected into cells and then integration into the production cell genome occurs infrequently and randomly. The transfection methods may be performed using methods well known in the art. For example, a stable transfection process may employ constructs which have been engineered to aid concatemerisation. In another example, the transfection process may be performed using calcium phosphate or commercially available formulations such as Lipofectamine™ 2000 CD (Invitrogen, CA), FuGENE® HD or polyethylenimine (PEI). Alternatively nucleotide sequences may be introduced into the production cell via electroporation. The skilled person will be aware of methods to encourage integration of the nucleotide sequences into production cells. For example, linearising a nucleic acid construct can help if it is naturally circular. Less random integration methodologies may involve the nucleic acid construct comprising of areas of shared homology with the endogenous chromosomes of the mammalian host cell to guide integration to a selected site within the endogenous genome. Furthermore, if recombination sites are present on the construct then these can be used for targeted recombination. For example, the nucleic acid construct may contain a IoxP site which allows for targeted integration when combined with Cre recombinase (i.e. using the Cre/lox system derived from P1 bacteriophage). Alternatively or additionally, the recombination site is an att site (e.g. from A phage), wherein the att site permits site-directed integration in the presence of a lambda integrase. This would allow the lentiviral genes to be targeted to a locus within the host cellular genome which allows for high and/or stable expression.

Other methods of targeted integration are well known in the art. For example, methods of inducing targeted cleavage of genomic DNA can be used to encourage targeted recombination at a selected chromosomal locus. These methods often involve the use of methods or systems to induce a double strand break (DSB) e.g. a nick in the endogenous genome to induce repair of the break by physiological mechanisms such as non-homologous end joining (NHEJ). Cleavage can occur through the use of specific nucleases such as engineered zinc finger nucleases (ZFN), transcription-activator like effector nucleases (TALENs), using CRISPR/Cas9 systems with an engineered crRNA/tracr RNA (‘single guide RNA’) to guide specific cleavage, and/or using nucleases based on the Argonaute system (e.g., from T. thermophilus).

Packaging/producer cell lines can be generated by integration of nucleotide sequences using methods of just lentiviral transduction or just nucleic acid transfection, or a combination of both can be used.

Methods for generating retroviral vectors from production cells and in particular the processing of retroviral vectors are described in WO 2009/153563.

In one embodiment, the production cell may comprise the RNA-binding protein (e.g. tryptophan RNA-binding attenuation protein, TRAP) and/or the Tet Repressor (TetR) protein or alternative regulatory proteins (e.g. CymR).

Production of lentiviral vector from production cells can be via transfection methods, from production from stable cell lines which can include induction steps (e.g. doxycycline induction) or via a combination of both. The transfection methods may be performed using methods well known in the art, and examples have been described previously.

Production cells, either packaging or producer cell lines or those transiently transfected with the lentiviral vector encoding components are cultured to increase cell and virus numbers and/or virus titres. Culturing a cell is performed to enable it to metabolize, and/or grow and/or divide and/or produce viral vectors of interest according to the invention. This can be accomplished by methods well known to persons skilled in the art, and includes but is not limited to providing nutrients for the cell, for instance in the appropriate culture media. The methods may comprise growth adhering to surfaces, growth in suspension, or combinations thereof. Culturing can be done for instance in tissue culture flasks, tissue culture multiwell plates, dishes, roller bottles, wave bags or in bioreactors, using batch, fed-batch, continuous systems and the like. In order to achieve large scale production of viral vector through cell culture it is preferred in the art to have cells capable of growing in suspension. Suitable conditions for culturing cells are known (see e.g. Tissue Culture, Academic Press, Kruse and Paterson, editors (1973), and R. I. Freshney, Culture of animal cells: A manual of basic technique, fourth edition (Wiley-Liss Inc., 2000, ISBN 0-471-34889-9).

Preferably cells are initially ‘bulked up’ in tissue culture flasks or bioreactors and subsequently grown in multi-layered culture vessels or large bioreactors (greater than 50 L) to generate the vector producing cells for use in the present invention.

Preferably cells are grown in a suspension mode to generate the vector producing cells for use in the present invention.

Lentiviral Vectors

Lentiviruses are part of a larger group of retroviruses. A detailed list of lentiviruses may be found in Coffin et al (1997) “Retroviruses” Cold Spring Harbour Laboratory Press Eds: J M Coffin, S M Hughes, H E Varmus pp 758-763). In brief, lentiviruses can be divided into primate and non-primate groups. Examples of primate lentiviruses include but are not limited to: the human immunodeficiency virus (HIV), the causative agent of human auto-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV), feline immunodeficiency virus (FIV), Maedi visna virus (MVV) and bovine immunodeficiency virus (BIV). In one embodiment, the lentiviral vector is derived from HIV-1, HIV-2, SIV, FIV, BIV, EIAV, CAEV or Visna lentivirus.

The lentivirus family differs from retroviruses in that lentiviruses have the capability to infect both dividing and non-dividing cells (Lewis et al (1992) EMBO J 11(8):3053-3058 and Lewis and Emerman (1994) J Virol 68 (1):510-516). In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Preferably, that component part is involved in the biological mechanisms by which the vector infects or transduces target cells and expresses NOI.

The lentiviral vector may be used to replicate the NOI in a compatible target cell in vitro. Thus, described herein is a method of making proteins in vitro by introducing a vector of the invention into a compatible target cell in vitro and growing the target cell under conditions which result in expression of the NOI. Protein and NOI may be recovered from the target cell by methods well known in the art. Suitable target cells include mammalian cell lines and other eukaryotic cell lines.

In some aspects the vectors may have “insulators”—genetic sequences that block the interaction between promoters and enhancers, and act as a barrier reducing read-through from an adjacent gene.

In one embodiment the insulator is present between one or more of the lentiviral nucleic acid sequences to prevent promoter interference and read-thorough from adjacent genes. If the insulators are present in the vector between one or more of the lentiviral nucleic acid sequences, then each of these insulated genes may be arranged as individual expression units.

The basic structure of retroviral and lentiviral genomes share many common features such as a 5′ LTR and a 3′ LTR, between or within which are located a packaging signal to enable the genome to be packaged, a primer binding site, integration sites to enable integration into a target cell genome and gag/pol and env genes encoding the packaging components—these are polypeptides required for the assembly of viral particles. Lentiviruses have additional features, such as the rev gene and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration, and transcription. LTRs also serve as enhancer-promoter sequences and can control the expression of the viral genes.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In a typical lentiviral vector as described herein, at least part of one or more protein coding regions essential for replication may be removed from the virus; for example, gag/pol and env may be absent or not functional. This makes the viral vector replication-defective.

The lentiviral vector may be derived from either a primate lentivirus (e.g. HIV-1) or a non-primate lentivirus (e.g. EIAV).

In general terms, a typical retroviral vector production system involves the separation of the viral genome from the essential viral packaging functions. These viral vector components are normally provided to the production cells on separate DNA expression cassettes (alternatively known as plasmids, expression plasmids, DNA constructs or expression constructs).

The vector genome comprises the NOI. Vector genomes typically require a packaging signal (ψ), the internal expression cassette harbouring the NOI, (optionally) a post-transcriptional element (PRE), typically a central polypurine tract (cppt), the 3′-ppu and a self-inactivating (SIN) LTR. The R-U5 regions are required for correct polyadenylation of both the vector genome RNA and NOI mRNA, as well as the process of reverse transcription. The vector genome may optionally include an open reading frame, as described in WO 2003/064665, which allows for vector production in the absence of rev.

The packaging functions include the gag/pol and env genes. These are required for the production of vector particles by the production cell. Providing these functions in trans to the genome facilitates the production of replication-defective viral vectors.

Production systems for gamma-retroviral vectors are typically 3-component systems requiring genome, gag/pol and env expression constructs. Production systems for HIV-1-based lentiviral vectors may additionally require the accessory gene rev to be provided and for the vector genome to include the rev-responsive element (RRE). EIAV-based lentiviral vectors do not require rev to be provided in trans if an open-reading frame (ORF) is present within the genome (see WO 2003/064665).

Usually both the “external” promoter (which drives the vector genome cassette) and “internal” promoter (which drives the NOI cassette) encoded within the vector genome cassette are strong eukaryotic or virus promoters, as are those driving the other vector system components. Examples of such promoters include CMV, EF1α, PGK, CAG, TK, SV40 and Ubiquitin promoters. Strong ‘synthetic’ promoters, such as those generated by DNA libraries (e.g. JeT promoter) may also be used to drive transcription. Alternatively, tissue-specific promoters such as rhodopsin (Rho), rhodopsin kinase (RhoK), cone-rod homeobox containing gene (CRX), neural retina-specific leucine zipper protein (NRL), Vitelliform Macular Dystrophy 2 (VMD2), Tyrosine hydroxylase, neuronal-specific neuronal-specific enolase (NSE) promoter, astrocyte-specific glial fibrillary acidic protein (GFAP) promoter, human al-antitrypsin (hAAT) promoter, phosphoenolpyruvate carboxykinase (PEPCK), liver fatty acid binding protein promoter, Flt-1 promoter, INF-β promoter, Mb promoter, SP-B promoter, SYN1 promoter, WASP promoter, SV40/hAlb promoter, SV40/CD43, SV40/CD45, NSE/RU5′ promoter, ICAM-2 promoter, GPIlb promoter, GFAP promoter, Fibronectin promoter, Endoglin promoter, Elastase-1 promoter, Desmin promoter, CD68 promoter, CD14 promoter and B29 promoter may be used to drive transcription.

Production of retroviral vectors involves either the transient co-transfection of the production cells with these DNA components or use of stable production cell lines wherein all the components are stably integrated within the production cell genome (e.g. Stewart H J, Fong-Wong L, Strickland I, Chipchase D, Kelleher M, Stevenson L, Thoree V, McCarthy J, Ralph G S, Mitrophanous K A and Radcliffe P A. (2011). Hum Gene Ther. March; 22 (3):357-69). An alternative approach is to use a stable packaging cell (into which the packaging components are stably integrated) and then transiently transfect in the vector genome plasmid as required (e.g. Stewart, H. J., M. A. Leroux-Carlucci, C. J. Sion, K. A. Mitrophanous and P. A. Radcliffe (2009). Gene Ther. June; 16 (6):805-14). It is also feasible that alternative, not complete, packaging cell lines could be generated (just one or two packaging components are stably integrated into the cell lines) and to generate vector the missing components are transiently transfected. The production cell may also express regulatory proteins such as a member of the tet repressor (TetR) protein group of transcription regulators (e.g. T-Rex, Tet-On, and Tet-Off), a member of the cumate inducible switch system group of transcription regulators (e.g. cumate repressor (CymR) protein), or an RNA-binding protein (e.g. TRAP-tryptophan-activated RNA-binding protein).

In one embodiment of the present invention, the viral vector is derived from EIAV. EIAV has the simplest genomic structure of the lentiviruses and is particularly preferred for use in the present invention. In addition to the gag/pol and env genes, EIAV encodes three other genes: tat, rev, and S2. Tat acts as a transcriptional activator of the viral LTR (Derse and Newbold (1993) Virology 194(2):530-536 and Maury et al (1994) Virology 200(2):632-642) and rev regulates and coordinates the expression of viral genes through rev-response elements (RRE) (Martarano et al. (1994) J Virol 68(5):3102-3111). The mechanisms of action of these two proteins are thought to be broadly similar to the analogous mechanisms in the primate viruses (Martarano et al. (1994) J Virol 68(5):3102-3111). The function of S2 is unknown. In addition, an EIAV protein, Ttm, has been identified that is encoded by the first exon of tat spliced to the env coding sequence at the start of the transmembrane protein. In an alternative embodiment of the present invention the viral vector is derived from HIV: HIV differs from EIAV in that it does not encode S2 but unlike EIAV it encodes vif, vpr, vpu and nef.

The term “recombinant retroviral or lentiviral vector” (RRV) refers to a vector with sufficient retroviral genetic information to allow packaging of an RNA genome, in the presence of packaging components, into a viral particle capable of transducing a target cell. Transduction of the target cell may include reverse transcription and integration into the target cell genome. The RRV carries non-viral coding sequences which are to be delivered by the vector to the target cell. A RRV is incapable of independent replication to produce infectious retroviral particles within the target cell. Usually the RRV lacks a functional gag/pol and/or env gene, and/or other genes essential for replication.

Preferably the RRV vector of the present invention has a minimal viral genome.

As used herein, the term “minimal viral genome” means that the viral vector has been manipulated so as to remove the non-essential elements whilst retaining the elements essential to provide the required functionality to infect, transduce and deliver a NOI to a target cell. Further details of this strategy can be found in WO 1998/17815 and WO 99/32646. A minimal EIAV vector lacks tat, rev and S2 genes and neither are these genes provided in trans in the production system. A minimal HIV vector lacks vif, vpr, vpu, tat and nef.

The expression plasmid used to produce the vector genome within a production cell may include transcriptional regulatory control sequences operably linked to the retroviral genome to direct transcription of the genome in a production cell/packaging cell. All 3rd generation lentiviral vectors are deleted in the 5′ U3 enhancer-promoter region, and transcription of the vector genome RNA is driven by heterologous promoter such as another viral promoter, for example the CMV promoter, as discussed below. This feature enables vector production independently of tat. Some lentiviral vector genomes require additional sequences for efficient virus production. For example, particularly in the case of HIV, RRE sequences may be included. However the requirement for RRE on the (separate) GagPol cassette (and dependence on rev which is provided in trans) may be reduced or eliminated by codon optimisation of the GagPol ORF. Further details of this strategy can be found in WO 2001/79518.

Alternative sequences which perform the same function as the rev/RRE system are also known. For example, a functional analogue of the rev/RRE system is found in the Mason Pfizer monkey virus. This is known as the constitutive transport element (CTE) and comprises an RRE-type sequence in the genome which is believed to interact with a factor in the infected cell. The cellular factor can be thought of as a rev analogue. Thus, CTE may be used as an alternative to the rev/RRE system. Any other functional equivalents of the Rev protein which are known or become available may be relevant to the invention. For example, it is also known that the Rex protein of HTLV-I can functionally replace the Rev protein of HIV-1. Rev and RRE may be absent or non-functional in the vector for use in the methods of the present invention; in the alternative rev and RRE, or functionally equivalent system, may be present.

As used herein, the term “functional substitute” means a protein or sequence having an alternative sequence which performs the same function as another protein or sequence. The term “functional substitute” is used interchangeably with “functional equivalent” and “functional analogue” herein with the same meaning.

SIN Vectors

The lentiviral vectors as described herein may be used in a self-inactivating (SIN) configuration in which the viral enhancer and promoter sequences have been deleted. SIN vectors can be generated and transduce non-dividing target cells in vivo, ex vivo or in vitro with an efficacy similar to that of non-SIN vectors. The transcriptional inactivation of the long terminal repeat (LTR) in the SIN provirus should prevent mobilisation of vRNA, and is a feature that further diminishes the likelihood of formation of replication-competent virus. This should also enable the regulated expression of genes from internal promoters by eliminating any cis-acting effects of the LTR.

By way of example, self-inactivating retroviral vector systems have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus. However, any promoter(s) internal to the LTRs in such vectors will still be transcriptionally active. This strategy has been employed to eliminate effects of the enhancers and promoters in the viral LTRs on transcription from internally placed genes. Such effects include increased transcription or suppression of transcription. This strategy can also be used to eliminate downstream transcription from the 3′ LTR into genomic DNA. This is of particular concern in human gene therapy where it is important to prevent the adventitious activation of any endogenous oncogene. Yu et al., (1986) PNAS 83: 3194-98; Marty et al., (1990) Biochimie 72: 885-7; Naviaux et al., (1996) J. Virol. 70: 5701-5; Iwakuma et al., (1999) Virol. 261: 120-32; Deglon et al., (2000) Human Gene Therapy 11: 179-90. SIN lentiviral vectors are described in U.S. Pat. Nos. 6,924,123 and 7,056,699.

Replication-Defective Lentiviral Vectors

In the genome of a replication-defective lentiviral vector the sequences of gag/pol and/or env may be mutated and/or not functional.

In a typical lentiviral vector as described herein, at least part of one or more coding regions for proteins essential for virus replication may be removed from the vector. This makes the viral vector replication-defective. Portions of the viral genome may also be replaced by a NOI in order to generate a vector comprising an NOI which is capable of transducing a non-dividing target cell and/or integrating its genome into the target cell genome.

In one embodiment the lentiviral vectors are non-integrating vectors as described in WO 2006/010834 and WO 2007/071994.

In a further embodiment the vectors have the ability to deliver a sequence which is devoid of or lacking viral RNA. In a further embodiment a heterologous binding domain (heterologous to gag) located on the RNA to be delivered and a cognate binding domain on Gag or GagPol can be used to ensure packaging of the RNA to be delivered. Both of these vectors are described in WO 2007/072056.

NOI and Polynucleotides

Polynucleotides of the invention may comprise DNA or RNA. They may be single-stranded or double-stranded. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

The polynucleotides may be modified by any method available in the art. Such modifications may be carried out in order to enhance the in vivo activity or lifespan of the polynucleotides of the invention.

Polynucleotides such as DNA polynucleotides may be produced recombinantly, synthetically or by any means available to those of skill in the art. They may also be cloned by standard techniques.

Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing PCR under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

Common Retroviral Vector Elements

Promoters and Enhancers

Expression of a NOI and polynucleotide may be controlled using control sequences for example transcription regulation elements or translation repression elements, which include promoters, enhancers and other expression regulation signals (e.g. tet repressor (TetR) system) or the Transgene Repression In vector Production cell system (TRiP) or other regulators of NOIs described herein.

Prokaryotic promoters and promoters functional in eukaryotic cells may be used. Tissue-specific or stimuli-specific promoters may be used. Chimeric promoters may also be used comprising sequence elements from two or more different promoters.

Suitable promoting sequences are strong promoters including those derived from the genomes of viruses, such as polyoma virus, adenovirus, fowlpox virus, bovine papilloma virus, avian sarcoma virus, cytomegalovirus (CMV), retrovirus and Simian Virus 40 (SV40), or from heterologous mammalian promoters, such as the actin promoter, EF1α, CAG, TK, SV40, ubiquitin, PGK or ribosomal protein promoter. Alternatively, tissue-specific promoters such as rhodopsin (Rho), rhodopsin kinase (RhoK), cone-rod homeobox containing gene (CRX), neural retina-specific leucine zipper protein (NRL), Vitelliform Macular Dystrophy 2 (VMD2), Tyrosine hydroxylase, neuronal-specific neuronal-specific enolase (NSE) promoter, astrocyte-specific glial fibrillary acidic protein (GFAP) promoter, human al-antitrypsin (hAAT) promoter, phosphoenolpyruvate carboxykinase (PEPCK), liver fatty acid binding protein promoter, Flt-1 promoter, INF-β promoter, Mb promoter, SP-B promoter, SYN1 promoter, WASP promoter, SV40/hAlb promoter, SV40/CD43, SV40/CD45, NSE/RU5′ promoter, ICAM-2 promoter, GPIlb promoter, GFAP promoter, Fibronectin promoter, Endoglin promoter, Elastase-1 promoter, Desmin promoter, CD68 promoter, CD14 promoter and B29 promoter may be used to drive transcription.

Transcription of a NOI may be increased further by inserting an enhancer sequence into the vector. Enhancers are relatively orientation- and position-independent; however, one may employ an enhancer from a eukaryotic cell virus, such as the SV40 enhancer and the CMV early promoter enhancer. The enhancer may be spliced into the vector at a position 5′ or 3′ to the promoter, but is preferably located at a site 5′ from the promoter.

The promoter can additionally include features to ensure or to increase expression in a suitable target cell. For example, the features can be conserved regions e.g. a Pribnow Box or a TATA box. The promoter may contain other sequences to affect (such as to maintain, enhance or decrease) the levels of expression of a nucleotide sequence. Suitable other sequences include the Sh1-intron or an ADH intron. Other sequences include inducible elements, such as temperature, chemical, light or stress inducible elements. Also, suitable elements to enhance transcription or translation may be present.

Regulators of NOIs

A complicating factor in the generation of retroviral packaging/producer cell lines and retroviral vector production is that constitutive expression of certain retroviral vector components and NOIs are cytotoxic leading to death of cells expressing these components and therefore inability to produce vector. Therefore, the expression of these components (e.g. gag-pol and envelope proteins such as VSV-G) can be regulated. The expression of other non-cytotoxic vector components, e.g. rev, can also be regulated to minimise the metabolic burden on the cell. The modular constructs and/or cells as described herein may comprise cytotoxic and/or non-cytotoxic vector components associated with at least one regulatory element. As used herein, the term “regulatory element” refers to any element capable of affecting, either increasing or decreasing, the expression of an associated gene or protein. A regulatory element includes a gene switch system, transcription regulation element and translation repression element.

A number of prokaryotic regulator systems have been adapted to generate gene switches in mammalian cells. Many retroviral packaging and producer cell lines have been controlled using gene switch systems (e.g. tetracycline and cumate inducible switch systems) thus enabling expression of one or more of the retroviral vector components to be switched on at the time of vector production. Gene switch systems include those of the (TetR) protein group of transcription regulators (e.g.T-Rex, Tet-On, and Tet-Off), those of the cumate inducible switch system group of transcription regulators (e.g. CymR protein) and those involving an RNA-binding protein (e.g. TRAP).

One such tetracycline-inducible system is the tetracycline repressor (TetR) system based on the T-REx™ system. By way of example, in such a system tetracycline operators (TetO2) are placed in a position such that the first nucleotide is 10 bp from the 3′ end of the last nucleotide of the TATATAA element of the human cytomegalovirus major immediate early promoter (hCMVp) then TetR alone is capable of acting as a repressor (Yao F, Svensjo T, Winkler T, Lu M, Eriksson C, Eriksson E. Tetracycline repressor, tetR, rather than the tetR-mammalian cell transcription factor fusion derivatives, regulates inducible gene expression in mammalian cells. 1998. Hum Gene Ther, 9: 1939-1950). In such a system the expression of the NOI can be controlled by a CMV promoter into which two copies of the TetO2 sequence have been inserted in tandem. TetR homodimers, in the absence of an inducing agent (tetracycline or its analogue doxycycline [dox]), bind to the TetO2 sequences and physically block transcription from the upstream CMV promoter. When present, the inducing agent binds to the TetR homodimers, causing allosteric changes such that it can no longer bind to the TetO2 sequences, resulting in gene expression. The TetR gene may be codon optimised as this may improve translation efficiency resulting in tighter control of TetO2 controlled gene expression.

The TRiP system is described in WO 2015/092440 and provides another way of repressing expression of the NOI in the production cells during vector production. The TRAP-binding sequence (e.g. TRAP-tbs) interaction forms the basis for a transgene protein repression system for the production of retroviral vectors, when a constitutive and/or strong promoter, including a tissue-specific promoter, driving the transgene is desirable and particularly when expression of the transgene protein in production cells leads to reduction in vector titres and/or elicits an immune response in vivo due to viral vector delivery of transgene-derived protein (Maunder et al, Nat Commun. (2017) Mar. 27; 8).

Briefly, the TRAP-tbs interaction forms a translational block, repressing translation of the transgene protein (Maunder et al, Nat Commun. (2017) Mar. 27; 8). The translational block is only effective in production cells and as such does not impede the DNA- or RNA-based vector systems. The TRiP system is able to repress translation when the transgene protein is expressed from a constitutive and/or strong promoter, including a tissue-specific promoter from single- or multi cistronic mRNA. It has been demonstrated that unregulated expression of transgene protein can reduce vector titres and affect vector product quality. Repression of transgene protein for both transient and stable PaCL/PCL vector production systems is beneficial for production cells to prevent a reduction in vector titres: where toxicity or molecular burden issues may lead to cellular stress; where transgene protein elicits an immune response in vivo due to viral vector delivery of transgene-derived protein; where the use of gene-editing transgenes may result in on/off target affects; where the transgene protein may affect vector and/or envelope glycoprotein exclusion.

Envelope and Pseudotyping

In one preferred aspect, the lentiviral vector as described herein has been pseudotyped. In this regard, pseudotyping can confer one or more advantages. For example, the env gene product of the HIV based vectors would restrict these vectors to infecting only cells that express a protein called CD4. But if the env gene in these vectors has been substituted with env sequences from other enveloped viruses, then they may have a broader infectious spectrum (Verma and Somia (1997) Nature 389(6648):239-242). By way of example, workers have pseudotyped an HIV based vector with the glycoprotein from VSV (Verma and Somia (1997) Nature 389(6648):239-242).

In another alternative, the Env protein may be a modified Env protein such as a mutant or engineered Env protein. Modifications may be made or selected to introduce targeting ability or to reduce toxicity or for another purpose (Valsesia-Wittman et al 1996 J Virol 70: 2056-64; Nilson et al (1996) Gene Ther 3(4):280-286; and Fielding et al (1998) Blood 91(5):1802-1809 and references cited therein).

The vector may be pseudotyped with any molecule of choice.

As used herein, “env” shall mean an endogenous lentiviral envelope or a heterologous envelope, as described herein.

VSV-G

The envelope glycoprotein (G) of Vesicular stomatitis virus (VSV), a rhabdovirus, is an envelope protein that has been shown to be capable of pseudotyping certain enveloped viruses and viral vector virions.

Its ability to pseudotype MoMLV-based retroviral vectors in the absence of any retroviral envelope proteins was first shown by Emi et al. (1991) Journal of Virology 65:1202-1207. WO 1994/294440 teaches that retroviral vectors may be successfully pseudotyped with VSV-G. These pseudotyped VSV-G vectors may be used to transduce a wide range of mammalian cells. More recently, Abe et al. (1998) J Virol 72(8) 6356-6361 teach that non-infectious retroviral particles can be made infectious by the addition of VSV-G.

Burns et al. (1993) Proc. Natl. Acad. Sci. USA 90:8033-7 successfully pseudotyped the retrovirus MLV with VSV-G and this resulted in a vector having an altered host range compared to MLV in its native form. VSV-G pseudotyped vectors have been shown to infect not only mammalian cells, but also cell lines derived from fish, reptiles and insects (Burns et al. (1993) ibid). They have also been shown to be more efficient than traditional amphotropic envelopes for a variety of cell lines (Yee et al., (1994) Proc. Natl. Acad. Sci. USA 91:9564-9568, Emi et al. (1991) Journal of Virology 65:1202-1207). VSV-G protein can be used to pseudotype certain retroviruses because its cytoplasmic tail is capable of interacting with the retroviral cores.

The provision of a non-retroviral pseudotyping envelope such as VSV-G protein gives the advantage that vector particles can be concentrated to a high titre without loss of infectivity (Akkina et al. (1996) J. Virol. 70:2581-5). Retrovirus envelope proteins are apparently unable to withstand the shearing forces during ultracentrifugation, probably because they consist of two non-covalently linked subunits. The interaction between the subunits may be disrupted by the centrifugation. In comparison the VSV glycoprotein is composed of a single unit. VSV-G protein pseudotyping can therefore offer potential advantages for both efficient target cell infection/transduction and during manufacturing processes.

WO 2000/52188 describes the generation of pseudotyped retroviral vectors, from stable producer cell lines, having vesicular stomatitis virus-G protein (VSV-G) as the membrane-associated viral envelope protein, and provides a gene sequence for the VSV-G protein.

Ross River Virus

The Ross River viral envelope has been used to pseudotype a non-primate lentiviral vector (FIV) and following systemic administration predominantly transduced the liver (Kang et al., 2002, J. Virol., 76:9378-9388). Efficiency was reported to be 20-fold greater than obtained with VSV-G pseudotyped vector, and caused less cytotoxicity as measured by serum levels of liver enzymes suggestive of hepatotoxicity.

Baculovirus GP64

The baculovirus GP64 protein has been shown to be an alternative to VSV-G for viral vectors used in the large-scale production of high-titre virus required for clinical and commercial applications (Kumar M, Bradow B P, Zimmerberg J (2003) Hum Gene Ther. 14(1):67-77). Compared with VSV-G-pseudotyped vectors, GP64-pseudotyped vectors have a similar broad tropism and similar native titres. Because, GP64 expression does not kill cells, HEK293T-based cell lines constitutively expressing GP64 can be generated.

Alternative Envelopes

Other envelopes which give reasonable titre when used to pseudotype EIAV include Mokola, Rabies, Ebola and LCMV (lymphocytic choriomeningitis virus). Intravenous infusion into mice of lentivirus pseudotyped with 4070A led to maximal gene expression in the liver.

Packaging Sequence

As utilized within the context of the present invention the term “packaging signal”, which is referred to interchangeably as “packaging sequence” or “psi”, is used in reference to the non-coding, cis-acting sequence required for encapsidation of retroviral RNA strands during viral particle formation. In HIV-1, this sequence has been mapped to loci extending from upstream of the major splice donor site (SD) to at least the gag start codon (some or all of the 5′ sequence of gag to nucleotide 688 may be included). In EIAV the packaging signal comprises the R region into the 5′ coding region of Gag.

As used herein, the term “extended packaging signal” or “extended packaging sequence” refers to the use of sequences around the psi sequence with further extension into the gag gene. The inclusion of these additional packaging sequences may increase the efficiency of insertion of vector RNA into viral particles.

Feline immunodeficiency virus (FIV) RNA encapsidation determinants have been shown to be discrete and non-continuous, comprising one region at the 5′ end of the genomic mRNA (R-U5) and another region that mapped within the proximal 311 nt of gag (Kaye et al., J Virol. October; 69(10):6588-92 (1995).

Internal Ribosome Entry Site (IRES)

Insertion of IRES elements allows expression of multiple coding regions from a single promoter (Adam et al (as above); Koo et al (1992) Virology 186:669-675; Chen et al 1993 J. Virol 67:2142-2148). IRES elements were first found in the non-translated 5′ ends of picornaviruses where they promote cap-independent translation of viral proteins (Jang et al (1990) Enzyme 44: 292-309). When located between open reading frames in an RNA, IRES elements allow efficient translation of the downstream open reading frame by promoting entry of the ribosome at the IRES element followed by downstream initiation of translation.

A review on IRES is presented by Mountford and Smith (TIG May 1995 vol 11, No 5:179-184). A number of different IRES sequences are known including those from encephalomyocarditis virus (EMCV) (Ghattas, I. R., et al., Mol. Cell. Biol., 11:5848-5859 (1991); BiP protein [Macejak and Sarnow, Nature 353:91 (1991)]; the Antennapedia gene of Drosophila (exons d and e) [Oh, et al., Genes & Development, 6:1643-1653 (1992)] as well as those in polio virus (PV) [Pelletier and Sonenberg, Nature 334: 320-325 (1988); see also Mountford and Smith, TIG 11, 179-184 (1985)].

IRES elements from PV, EMCV and swine vesicular disease virus have previously been used in retroviral vectors (Coffin et al, as above).

The term “IRES” includes any sequence or combination of sequences which work as or improve the function of an IRES. The IRES(s) may be of viral origin (such as EMCV IRES, PV IRES, or FMDV 2A-like sequences) or cellular origin (such as FGF2 IRES, NRF IRES, Notch 2 IRES or EIF4 IRES).

In order for the IRES to be capable of initiating translation of each polynucleotide it should be located between or prior to the polynucleotides in the modular construct.

The nucleotide sequences utilised for development of stable cell lines require the addition of selectable markers for selection of cells where stable integration has occurred. These selectable markers can be expressed as a single transcription unit within the nucleotide sequence or it may be preferable to use IRES elements to initiate translation of the selectable marker in a polycistronic message (Adam et al 1991 J. Virol. 65, 4985).

Genetic Orientation and Insulators

It is well known that nucleic acids are directional and this ultimately affects mechanisms such as transcription and replication in the cell. Thus genes can have relative orientations with respect to one another when part of the same nucleic acid construct.

In certain embodiments of the present invention, at least two nucleic acid sequences present at the same locus in the cell or construct can be in a reverse and/or alternating orientations. In other words, in certain embodiments of the invention at this particular locus, the pair of sequential genes will not have the same orientation. This can help prevent both transcriptional and translational read-through when the region is expressed within the same physical location of the host cell.

Having the alternating orientations benefits retroviral vector production when the nucleic acids required for vector production are based at the same genetic locus within the cell. This in turn can also improve the safety of the resulting constructs in preventing the generation of replication-competent retroviral vectors.

When nucleic acid sequences are in reverse and/or alternating orientations the use of insulators can prevent inappropriate expression or silencing of a NOI from its genetic surroundings.

The term “insulator” refers to a class of DNA sequence elements that when bound to insulator-binding proteins possess an ability to protect genes from surrounding regulator signals. There are two types of insulators: an enhancer blocking function and a chromatin barrier function. When an insulator is situated between a promoter and an enhancer, the enhancer-blocking function of the insulator shields the promoter from the transcription-enhancing influence of the enhancer (Geyer and Corces 1992; Kellum and Schedl 1992). The chromatin barrier insulators function by preventing the advance of nearby condensed chromatin which would lead to a transcriptionally active chromatin region turning into a transcriptionally inactive chromatin region and resulting in silencing of gene expression. Insulators which inhibit the spread of heterochromatin, and thus gene silencing, recruit enzymes involved in histone modifications to prevent this process (Yang J, Corces VG. 2011; 110:43-76; Huang, Li et al. 2007; Dhillon, Raab et al. 2009). An insulator can have one or both of these functions and the chicken β-globin insulator (cHS4) is one such example. This insulator is the most extensively studied vertebrate insulator, is highly rich in G+C and has both enhancer-blocking and heterochromatic barrier functions (Chung J H, Whitely M, Felsenfeld G. Cell. 1993; 74:505-514). Other such insulators with enhancer blocking functions are not limited to but include the following: human β-globin insulator 5 (HS5), human β-globin insulator 1 (HS1), and chicken β-globin insulator (cHS3) (Farrell CM1, West A G, Felsenfeld G., Mol Cell Biol. 2002 June; 22(11):3820-31; J Ellis et al. EMBO J. 1996 Feb. 1; 15(3): 562-568). In addition to reducing unwanted distal interactions the insulators also help to prevent promoter interference (i.e. where the promoter from one transcription unit impairs expression of an adjacent transcription unit) between adjacent retroviral nucleic acid sequences. If the insulators are used between each of the retroviral vector nucleic acid sequences, then the reduction of direct read-through will help prevent the formation of replication-competent retroviral vector particles.

The insulator may be present between each of the retroviral nucleic acid sequences. In one embodiment, the use of insulators prevents promoter-enhancer interactions from one NOI expression cassette interacting with another NOI expression cassette in a nucleotide sequence encoding vector components.

An insulator may be present between the vector genome and gag-pol sequences. This therefore limits the likelihood of the production of a replication-competent retroviral vector and ‘wild-type’ like RNA transcripts, improving the safety profile of the construct. The use of insulator elements to improve the expression of stably integrated multigene vectors is cited in Moriarity et al, Nucleic Acids Res. 2013 April; 41(8):e92.

Vector Titre

The skilled person will understand that there are a number of different methods of determining the titre of lentiviral vectors. Titre is often described as transducing units/mL (TU/mL). Titre may be increased by increasing the number of vector particles and by increasing the specific activity of a vector preparation.

Therapeutic Use

The lentiviral vector as described herein or a cell or tissue transduced with the lentiviral vector as described herein may be used in medicine.

In addition, the lentiviral vector as described herein, a production cell of the invention or a cell or tissue transduced with the lentiviral vector as described herein may be used for the preparation of a medicament to deliver a nucleotide of interest to a target site in need of the same. Such uses of the lentiviral vector or transduced cell of the invention may be for therapeutic or diagnostic purposes, as described previously.

Accordingly, there is provided a cell transduced by the lentiviral vector as described herein.

A “cell transduced by a viral vector particle” or a “cell transduced by a lentiviral vector” is to be understood as a cell, in particular a target cell, into which the nucleic acid carried by the viral vector particle has been transferred.

In one embodiment of the invention, the nucleotide of interest is translated in a target cell which lacks TRAP.

“Target cell” is to be understood as a cell in which it is desired to express the NOI. The NOI may be introduced into the target cell using a viral vector of the present invention. Delivery to the target cell may be performed in vivo, ex vivo or in vitro.

In a preferred embodiment, the nucleotide of interest gives rise to a therapeutic effect.

The NOI may have a therapeutic or diagnostic application. Suitable NOIs include, but are not limited to sequences encoding enzymes, co-factors, cytokines, chemokines, hormones, antibodies, anti-oxidant molecules, engineered immunoglobulin-like molecules, single chain antibodies, fusion proteins, immune co-stimulatory molecules, immunomodulatory molecules, chimeric antigen receptors a transdomain negative mutant of a target protein, toxins, conditional toxins, antigens, transcription factors, structural proteins, reporter proteins, subcellular localization signals, tumour suppressor proteins, growth factors, membrane proteins, receptors, vasoactive proteins and peptides, anti-viral proteins and ribozymes, and derivatives thereof (such as derivatives with an associated reporter group). The NOIs may also encode micro-RNA. Without wishing to be bound by theory, it is believed that the processing of micro-RNA will be inhibited by TRAP.

In one embodiment, the NOI may be useful in the treatment of a neurodegenerative disorder.

In another embodiment, the NOI may be useful in the treatment of Parkinson's disease.

In another embodiment, the NOI may encode an enzyme or enzymes involved in dopamine synthesis. For example, the enzyme may be one or more of the following: tyrosine hydroxylase, GTP-cyclohydrolase I and/or aromatic amino acid dopa decarboxylase. The sequences of all three genes are available (GenBank® Accession Nos. X05290, U19523 and M76180, respectively).

In another embodiment, the NOI may encode the vesicular monoamine transporter 2 (VMAT2). In an alternative embodiment the viral genome may comprise a NOI encoding aromatic amino acid dopa decarboxylase and a NOI encoding VMAT2. Such a genome may be used in the treatment of Parkinson's disease, in particular in conjunction with peripheral administration of L-DOPA.

In another embodiment the NOI may encode a therapeutic protein or combination of therapeutic proteins.

In another embodiment, the NOI may encode a protein or proteins selected from the group consisting of glial cell derived neurotophic factor (GDNF), brain derived neurotrophic factor (BDNF), ciliary neurotrophic factor (CNTF), neurotrophin-3 (NT-3), acidic fibroblast growth factor (aFGF), basic fibroblast growth factor (bFGF), interleukin-1 beta (IL-1β), tumor necrosis factor alpha (TNF-α), insulin growth factor-2, VEGF-A, VEGF-B, VEGF-C/VEGF-2, VEGF-D, VEGF-E, PDGF-A, PDGF-B, hetero- and homo-dimers of PDFG-A and PDFG-B.

In another embodiment, the NOI may encode an anti-angiogenic protein or anti-angiogenic proteins selected from the group consisting of angiostatin, endostatin, platelet factor 4, pigment epithelium derived factor (PEDF), placental growth factor, restin, interferon-α, interferon-inducible protein, gro-beta and tubedown-1, interleukin(IL)-1, IL-12, retinoic acid, anti-VEGF antibodies or fragments/variants thereof such as aflibercept, thrombospondin, VEGF receptor proteins such as those described in U.S. Pat. Nos. 5,952,199 and 6,100,071, and anti-VEGF receptor antibodies.

In another embodiment, the NOI may encode anti-inflammatory proteins, antibodies or fragment/variants of proteins or antibodies selected from the group consisting of NF-kB inhibitors, IL1beta inhibitors, TGFbeta inhibitors, IL-6 inhibitors, IL-23 inhibitors, IL-18 inhibitors, Tumour necrosis factor alpha and Tumour necrosis factor beta, Lymphotoxin alpha and Lymphotoxin beta, LIGHT inhibitors, alpha synuclein inhibitors, Tau inhibitors, beta amyloid inhibitors, IL-17 inhibitors,

In another embodiment the NOI may encode cystic fibrosis transmembrane conductance regulator (CFTR).

In another embodiment the NOI may encode a protein normally expressed in an ocular cell.

In another embodiment, the NOI may encode a protein normally expressed in a photoreceptor cell and/or retinal pigment epithelium cell.

In another embodiment, the NOI may encode a protein selected from the group comprising RPE65, arylhydrocarbon-interacting receptor protein like 1 (AIPL1), CRB1, lecithin retinal acetyltransferace (LRAT), photoreceptor-specific homeo box (CRX), retinal guanylate cyclise (GUCY2D), RPGR interacting protein 1 (RPGRIP1), LCA2, LCA3, LCA5, dystrophin, PRPH2, CNTF, ABCR/ABCA4, EMP1, TIMP3, MERTK, ELOVL4, MYO7A, USH2A, VMD2, RLBP1, COX-2, FPR, harmonin, Rab escort protein 1, CNGB2, CNGA3, CEP 290, RPGR, RS1, RP1, PRELP, glutathione pathway enzymes and opticin.

In other embodiments, the NOI may encode the human clotting Factor VIII or Factor IX.

In other embodiments, the NOI may encode protein or proteins involved in metabolism selected from the group comprising phenylalanine hydroxylase (PAH), Methylmalonyl CoA mutase, Propionyl CoA carboxylase, Isovaleryl CoA dehydrogenase, Branched chain ketoacid dehydrogenase complex, Glutaryl CoA dehydrogenase, Acetyl CoA carboxylase, propionyl CoA carboxylase, 3 methyl crotonyl CoA carboxylase, pyruvate carboxylase, carbamoyl-phophate synthase ammonia, ornithine transcarbamylase, glucosylceramidase beta, alpha galactosidase A, glucosylceramidase beta, cystinosin, glucosamine(N-acetyl)-6-sulfatase, N-acetyl-alpha-glucosaminidase, N-sulfoglucosamine sulfohydrolase, Galactosamine-6 sulfatase, arylsulfatase A, cytochrome B-245 beta, ABCD1, ornithine carbamoyltransferase, argininosuccinate synthase, argininosuccinate lysase, arginase 1, alanine glycoxhylate amino transferase, ATP-binding cassette, sub-family B members.

In other embodiments, the NOI may encode a chimeric antigen receptor (CAR) or a T cell receptor (TCR). In one embodiment, the CAR is an anti-5T4 CAR. In other embodiments, the NOI may encode B-cell maturation antigen (BCMA), CD19, CD22, CD20, CD138, CD30, CD33, CD123, CD70, prostate specific membrane antigen (PSMA), Lewis Y antigen (LeY), Tyrosine-protein kinase transmembrane receptor (ROR1), Mucin 1, cell surface associated (Muc1), Epithelial cell adhesion molecule (EpCAM), endothelial growth factor receptor (EGFR), insulin, protein tyrosine phosphatase, non-receptor type 22, interleukin 2 receptor, alpha, interferon induced with helicase C domain 1, human epidermal growth factor receptor (HER2), glypican 3 (GPC3), disialoganglioside (GD2), mesiothelin, vesicular endothelial growth factor receptor 2 (VEGFR2).

In other embodiments, the NOI may encode a chimeric antigen receptor (CAR) against NKG2D ligands selected from the group comprising ULBP1, 2 and 3, H60, Rae-1a, b, g, d, MICA, MICB.

In further embodiments the NOI may encode SGSH, SUMF1, GAA, the common gamma chain (CD132), adenosine deaminase, WAS protein, globins, alpha galactosidase A, δ-aminolevulinate (ALA) synthase, δ-aminolevulinate dehydratase (ALAD), Hydroxymethylbilane (HMB) synthase, Uroporphyrinogen (URO) synthase, Uroporphyrinogen (URO) decarboxylase, Coproporphyrinogen (COPRO) oxidase, Protoporphyrinogen (PROTO) oxidase, Ferrochelatase, α-L-iduronidase, Iduronate sulfatase, Heparan sulfamidase, N-acetylglucosaminidase, Heparan-α-glucosaminide N-acetyltransferase, 3 N-acetylglucosamine 6-sulfatase, Galactose-6-sulfate sulfatase, β-galactosidase, N-acetylgalactosamine-4-sulfatase, β-glucuronidase and Hyaluronidase.

In addition to the NOI the vector may also comprise or encode a siRNA, shRNA, or regulated shRNA. (Dickins et al. (2005) Nature Genetics 37: 1289-1295, Silva et al. (2005) Nature Genetics 37:1281-1288).

Indications

The vectors, including retroviral and AAV vectors, according to the present invention may be used to deliver one or more NOI(s) useful in the treatment of the disorders listed in WO 1998/05635, WO 1998/07859, WO 1998/09985. The nucleotide of interest may be DNA or RNA. Examples of such diseases are given below:
A disorder which responds to cytokine and cell proliferation/differentiation activity; immunosuppressant or immunostimulant activity (e.g. for treating immune deficiency, including infection with human immunodeficiency virus, regulation of lymphocyte growth; treating cancer and many autoimmune diseases, and to prevent transplant rejection or induce tumour immunity); regulation of haematopoiesis (e.g. treatment of myeloid or lymphoid diseases); promoting growth of bone, cartilage, tendon, ligament and nerve tissue (e.g. for healing wounds, treatment of burns, ulcers and periodontal disease and neurodegeneration); inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); anti-inflammatory activity (for treating, for example, septic shock or Crohn's disease); macrophage inhibitory and/or T cell inhibitory activity and thus, anti-inflammatory activity; anti-immune activity (i.e. inhibitory effects against a cellular and/or humoral immune response, including a response not associated with inflammation); inhibition of the ability of macrophages and T cells to adhere to extracellular matrix components and fibronectin, as well as up-regulated fas receptor expression in T cells.
Malignancy disorders, including cancer, leukaemia, benign and malignant tumour growth, invasion and spread, angiogenesis, metastases, ascites and malignant pleural effusion.
Autoimmune diseases including arthritis, including rheumatoid arthritis, hypersensitivity, allergic reactions, asthma, systemic lupus erythematosus, collagen diseases and other diseases.
Vascular diseases including arteriosclerosis, atherosclerotic heart disease, reperfusion injury, cardiac arrest, myocardial infarction, vascular inflammatory disorders, respiratory distress syndrome, cardiovascular effects, peripheral vascular disease, migraine and aspirin-dependent anti-thrombosis, stroke, cerebral ischaemia, ischaemic heart disease or other diseases.
Diseases of the gastrointestinal tract including peptic ulcer, ulcerative colitis, Crohn's disease and other diseases.
Hepatic diseases including hepatic fibrosis, liver cirrhosis.
Inherited metabolic disorders including phenylketonuria PKU, Wilson disease, organic acidemias, urea cycle disorders, cholestasis, and other diseases.
Renal and urologic diseases including thyroiditis or other glandular diseases, glomerulonephritis or other diseases.
Ear, nose and throat disorders including otitis or other oto-rhino-laryngological diseases, dermatitis or other dermal diseases.
Dental and oral disorders including periodontal diseases, periodontitis, gingivitis or other dental/oral diseases.
Testicular diseases including orchitis or epididimo-orchitis, infertility, orchidal trauma or other testicular diseases.
Gynaecological diseases including placental dysfunction, placental insufficiency, habitual abortion, eclampsia, pre-eclampsia, endometriosis and other gynaecological diseases.
Ophthalmologic disorders such as Leber Congenital Amaurosis (LCA) including LCA10, posterior uveitis, intermediate uveitis, anterior uveitis, conjunctivitis, chorioretinitis, uveoretinitis, optic neuritis, glaucoma, including open angle glaucoma and juvenile congenital glaucoma, intraocular inflammation, e.g. retinitis or cystoid macular oedema, sympathetic ophthalmia, scleritis, retinitis pigmentosa, macular degeneration including age related macular degeneration (AMD) and juvenile macular degeneration including Best Disease, Best vitelliform macular degeneration, Stargardt's Disease, Usher's syndrome, Doyne's honeycomb retinal dystrophy, Sorby's Macular Dystrophy, Juvenile retinoschisis, Cone-Rod Dystrophy, Corneal Dystrophy, Fuch's Dystrophy, Leber's congenital amaurosis, Leber's hereditary optic neuropathy (LHON), Adie syndrome, Oguchi disease, degenerative fondus disease, ocular trauma, ocular inflammation caused by infection, proliferative vitreo-retinopathies, acute ischaemic optic neuropathy, excessive scarring, e.g. following glaucoma filtration operation, reaction against ocular implants, corneal transplant graft rejection, and other ophthalmic diseases, such as diabetic macular oedema, retinal vein occlusion, RLBP1-associated retinal dystrophy, choroideremia and achromatopsia.
Neurological and neurodegenerative disorders including Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, strokes, post-polio syndrome, psychiatric disorders, myelitis, encephalitis, subacute sclerosing pan-encephalitis, encephalomyelitis, acute neuropathy, subacute neuropathy, chronic neuropathy, Fabry disease, Gaucher disease, Cystinosis, Pompe disease, metachromatic leukodystrophy, Wiscott Aldrich Syndrome, adrenoleukodystrophy, beta-thalassemia, sickle cell disease, Guillaim-Barre syndrome, Sydenham chorea, myasthenia gravis, pseudo-tumour cerebri,
Down's Syndrome, Huntington's disease, CNS compression or CNS trauma or infections of the CNS, muscular atrophies and dystrophies, diseases, conditions or disorders of the central and peripheral nervous systems, motor neuron disease including amyotropic lateral sclerosis, spinal muscular atropy, spinal cord and avulsion injury.
Other diseases and conditions such as cystic fibrosis, mucopolysaccharidosis including Sanfilipo syndrome A, Sanfilipo syndrome B, Sanfilipo syndrome C, Sanfilipo syndrome D, Hunter syndrome, Hurler-Scheie syndrome, Morquio syndrome, ADA-SCID, X-linked SCID, X-linked chronic granulomatous disease, porphyria, haemophilia A, haemophilia B, post-traumatic inflammation, haemorrhage, coagulation and acute phase response, cachexia, anorexia, acute infection, septic shock, infectious diseases, diabetes mellitus, complications or side effects of surgery, bone marrow transplantation or other transplantation complications and/or side effects, complications and side effects of gene therapy, e.g. due to infection with a viral carrier, or AIDS, to suppress or inhibit a humoral and/or cellular immune response, for the prevention and/or treatment of graft rejection in cases of transplantation of natural or artificial cells, tissue and organs such as cornea, bone marrow, organs, lenses, pacemakers, natural or artificial skin tissue.

siRNA, Micro-RNA and shRNA

In certain other embodiments, the NOI comprises a micro-RNA. Micro-RNAs are a very large group of small RNAs produced naturally in organisms, at least some of which regulate the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as an ˜70 nt precursor, which is post-transcriptionally processed into a mature ˜21 nt form. Both let-7 and lin-4 are transcribed as hairpin RNA precursors which are processed to their mature forms by Dicer enzyme.

In addition to the NOI the vector may also comprise or encode a siRNA, shRNA, or regulated shRNA (Dickins et al. (2005) Nature Genetics 37: 1289-1295, Silva et al. (2005) Nature Genetics 37:1281-1288).

Post-transcriptional gene silencing (PTGS) mediated by double-stranded RNA (dsRNA) is a conserved cellular defence mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi) (Ralph et al. (2005) Nature Medicine 11:429-433). The mechanism of RNAi involves the processing of long dsRNAs into duplexes of about 21-25 nucleotide (nt) RNAs. These products are called small interfering or silencing RNAs (siRNAs) which are the sequence-specific mediators of mRNA degradation. In differentiated mammalian cells, dsRNA >30 bp has been found to activate the interferon response leading to shut-down of protein synthesis and non-specific mRNA degradation (Stark et al., Annu Rev Biochem 67:227-64 (1998)). However this response can be bypassed by using 21 nt siRNA duplexes (Elbashir et al., EMBO J. December 3; 20(23):6877-88 (2001), Hutvagner et al., Science. August 3, 293(5531):834-8. Eupub July 12 (2001)) allowing gene function to be analysed in cultured mammalian cells.

### PHARMACEUTICAL COMPOSITIONS

The present invention provides a pharmaceutical composition comprising the lentiviral vector as described herein or a cell or tissue transduced with the viral vector as described herein, in combination with a pharmaceutically acceptable carrier, diluent or excipient.

The present invention provides a pharmaceutical composition for treating an individual by gene therapy, wherein the composition comprises a therapeutically effective amount of a lentiviral vector. The pharmaceutical composition may be for human or animal usage.

The composition may comprise a pharmaceutically acceptable carrier, diluent, excipient or adjuvant. The choice of pharmaceutical carrier, excipient or diluent can be made with regard to the intended route of administration and standard pharmaceutical practice. The pharmaceutical compositions may comprise, or be in addition to, the carrier, excipient or diluent any suitable binder(s), lubricant(s), suspending agent(s), coating agent(s), solubilising agent(s) and other carrier agents that may aid or increase vector entry into the target site (such as for example a lipid delivery system).

Where appropriate, the composition can be administered by any one or more of inhalation; in the form of a suppository or pessary; topically in the form of a lotion, solution, cream, ointment or dusting powder; by use of a skin patch; orally in the form of tablets containing excipients such as starch or lactose, or in capsules or ovules either alone or in admixture with excipients, or in the form of elixirs, solutions or suspensions containing flavouring or colouring agents; or they can be injected parenterally, for example intracavernosally, intravenously, intramuscularly, intracranially, intraoccularly intraperitoneally, or subcutaneously. For parenteral administration, the compositions may be best used in the form of a sterile aqueous solution which may contain other substances, for example enough salts or monosaccharides to make the solution isotonic with blood. For buccal or sublingual administration, the compositions may be administered in the form of tablets or lozenges which can be formulated in a conventional manner.

The lentiviral vector as described herein may also be used to transduce target cells or target tissue ex vivo prior to transfer of said target cell or tissue into a patient in need of the same. An example of such cell may be autologous T cells and an example of such tissue may be a donor cornea.

Variants, Derivatives, Analogues, Homologues and Fragments

In addition to the specific proteins and nucleotides mentioned herein, the present invention also encompasses the use of variants, derivatives, analogues, homologues and fragments thereof.

In the context of the present invention, a variant of any given sequence is a sequence in which the specific sequence of residues (whether amino acid or nucleic acid residues) has been modified in such a manner that the polypeptide or polynucleotide in question retains at least one of its endogenous functions. A variant sequence can be obtained by addition, deletion, substitution, modification, replacement and/or variation of at least one residue present in the naturally-occurring protein.

The term “derivative” as used herein, in relation to proteins or polypeptides of the present invention includes any substitution of, variation of, modification of, replacement of, deletion of and/or addition of one (or more) amino acid residues from or to the sequence providing that the resultant protein or polypeptide retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3 to 10 or 20 substitutions provided that the modified sequence retains the required activity or ability. Amino acid substitutions may include the use of non-naturally occurring analogues.

Proteins used in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The term “homologue” means an entity having a certain homology with the wild type amino acid sequence and the wild type nucleotide sequence. The term “homology” can be equated with “identity”.

In the present context, a homologous sequence is taken to include an amino acid sequence which may be at least 50%, 55%, 65%, 75%, 85% or 90% identical, preferably at least 95%, 97 or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence which may be at least 50%, 55%, 65%, 75%, 85% or 90% identical, preferably at least 95%, 97%, 98% or 99% identical to the subject sequence. Although homology can also be considered in terms of similarity, in the context of the present invention it is preferred to express homology in terms of sequence identity.

Homology comparisons can be conducted by eye, or more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Research 12:387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol Lett (1999) 174(2):247-50; FEMS Microbiol Lett (1999) 177(1):187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate percentage homology, preferably percentage sequence identity. The software usually does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the break. The DNA is then expressed in accordance with the invention to make the encoded protein. These methods are only illustrative of the numerous standard techniques known in the art for manipulation of DNA sequences and other known techniques may also be used.

Codon Optimisation

The polynucleotides used in the present invention (including the NOI and/or components of the vector production system) may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Many viruses, including retroviruses, use a large number of rare codons and changing these to correspond to commonly used mammalian codons, increases expression of a gene of interest, e.g. a NOI or packaging components in mammalian production cells, can be achieved. Codon usage tables are known in the art for mammalian cells, as well as for a variety of other organisms.

Codon optimisation of viral vector components has a number of other advantages. By virtue of alterations in their sequences, the nucleotide sequences encoding the packaging components of the viral particles required for assembly of viral particles in the producer cells/packaging cells have RNA instability sequences (INS) eliminated from them. At the same time, the amino acid sequence coding sequence for the packaging components is retained so that the viral components encoded by the sequences remain the same, or at least sufficiently similar that the function of the packaging components is not compromised. In lentiviral vectors codon optimisation also overcomes the Rev/RRE requirement for export, rendering optimised sequences Rev-independent. Codon optimisation also reduces homologous recombination between different constructs within the vector system (for example between the regions of overlap in the gag-pol and env open reading frames). The overall effect of codon optimisation is therefore a notable increase in viral titre and improved safety.

In one embodiment only codons relating to INS are codon optimised. However, in a much more preferred and practical embodiment, the sequences are codon optimised in their entirety, with some exceptions, for example the sequence encompassing the frameshift site of gag-pol (see below).

The gag-pol gene of lentiviral vectors comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimised. Retaining this fragment will enable more efficient expression of the Gag-Pol proteins. For EIAV the beginning of the overlap has been taken to be nt 1262 (where nucleotide 1 is the A of the gag ATG) and the end of the overlap to be nt 1461. In order to ensure that the frameshift site and the gag-pol overlap are preserved, the wild type sequence has been retained from nt 1156 to 1465.

Derivations from optimal codon usage may be made, for example, in order to accommodate convenient restriction sites, and conservative amino acid changes may be introduced into the Gag-Pol proteins.

In one embodiment, codon optimisation is based on lightly expressed mammalian genes. The third and sometimes the second and third base may be changed.

Due to the degenerate nature of the genetic code, it will be appreciated that numerous gag-pol sequences can be achieved by a skilled worker. Also there are many retroviral variants described which can be used as a starting point for generating a codon-optimised gag-pol sequence. Lentiviral genomes can be quite variable. For example there are many quasi-species of HIV-1 which are still functional. This is also the case for EIAV. These variants may be used to enhance particular parts of the transduction process. Examples of HIV-1 variants may be found at the HIV Databases operated by Los Alamos National Security, LLC at http://hiv-web.lanl.gov. Details of EIAV clones may be found at the National Center for Biotechnology Information (NCBI) database located at http://www.ncbi.nlm.nih.gov.

The strategy for codon-optimised gag-pol sequences can be used in relation to any retrovirus. This would apply to all lentiviruses, including EIAV, FIV, BIV, CAEV, VMR, SIV, HIV-1 and HIV-2. In addition this method could be used to increase expression of genes from HTLV-1, HTLV-2, HFV, HSRV and human endogenous retroviruses (HERV), MLV and other retroviruses.

Codon optimisation can render gag-pol expression Rev-independent. In order to enable the use of anti-rev or RRE factors in the lentiviral vector, however, it would be necessary to render the viral vector generation system totally Rev/RRE-independent. Thus, the genome also needs to be modified. This is achieved by optimising vector genome components. Advantageously, these modifications also lead to the production of a safer system absent of all additional proteins both in the producer and in the transduced cell.

Nucleotide Numbering

In the present invention, a specific numbering of nucleotide positions in the modified RREs and modified nucleotide sequences encoding gag used in the present invention may be employed. By alignment of the nucleotide sequence of a sample RRE with the RRE defined in SEQ ID NO: 2, it is possible to allot a number to a nucleotide position in said sample RRE which corresponds with the nucleotide position or numbering of the nucleotide sequence shown in SEQ ID NO: 2 of the present disclosure. Similarly, by alignment of the nucleotide sequence of a sample nucleotide sequence encoding gag with the nucleotide sequence encoding gag defined in SEQ ID NO: 6, it is possible to allot a number to a nucleotide position in said sample nucleotide sequence encoding gag which corresponds with the nucleotide position or numbering of the nucleotide sequence shown in SEQ ID NO: 6 of the present disclosure. Similarly, by alignment of the nucleotide sequence of a sample WPRE with the WPRE defined in SEQ ID NO: 11, it is possible to allot a number to a nucleotide position in said sample WPRE which corresponds with the nucleotide position or numbering of the nucleotide sequence shown in SEQ ID NO: 11 of the present disclosure.

An alternative way of describing the nucleotide numbering used in this application is to say that nucleotide positions are identified by those ‘corresponding’ to a particular position in the nucleotide sequence shown in SEQ ID NO: 2, SEQ ID NO: 6 or SEQ ID NO: 11. This is not to be interpreted as meaning the sequences of the present invention must include the nucleotide sequence shown in SEQ ID NO: 2, SEQ ID NO: 6 or SEQ ID NO: 11. A skilled person will readily appreciate that RREs and nucleotide sequences encoding gag vary among different lentiviral vectors. Reference to the nucleotide sequence shown in SEQ ID NO: 2, SEQ ID NO: 6 or SEQ ID NO: 11 is used merely to enable identification of a particular nucleotide location within any particular RRE or nucleotide sequence encoding gag. Such nucleotide locations can be routinely identified using sequence alignment programs, the use of which are well known in the art.

RNA Splicing

The major splice donor site in the lentiviral vector genome as described herein may be inactivated. The cryptic splice donor site 3′ to the major splice donor site in the lentiviral vector genome as described herein may be inactivated. The major splice donor site and the cryptic splice donor site 3′ to the major splice donor site in the lentiviral vector genome as described herein may be inactivated.

RNA splicing is catalysed by a large RNA-protein complex called the spliceosome, which is comprised of five small nuclear ribonucleoproteins (snRNPs). The borders between introns and exons are marked by specific nucleotide sequences within a pre-mRNA, which delineate where splicing will occur. Such boundaries are referred to as “splice sites.” The term “splice site” refers to polynucleotides that are capable of being recognized by the splicing machinery of a eukaryotic cell as suitable for being cut and/or ligated to another splice site.

Splice sites allow for the excision of introns present in a pre-mRNA transcript. Typically, the 5′ splice boundary is referred to as the “splice donor site” or the “5′ splice site,” and the 3′ splice boundary is referred to as the “splice acceptor site” or the “3′ splice site.” Splice sites include, for example, naturally occurring splice sites, engineered or synthetic splice sites, canonical or consensus splice sites, and/or non-canonical splice sites, for example, cryptic splice sites.

The terms “canonical splice site” or “consensus splice site” may be used interchangeably and refer to splice sites that are conserved across species.

Consensus sequences for the 5′ donor splice site and the 3′ acceptor splice site used in eukaryotic RNA splicing are well known in the art. These consensus sequences include nearly invariant dinucleotides at each end of the intron: GT at the 5′ end of the intron, and AG at the 3′ end of an intron.

The canonical splice donor site consensus sequence may be (for DNA) AG/GTRAGT (where A is adenosine, T is thymine, G is guanine, C is cytosine, R is a purine and “/” indicates the cleavage site). This conforms to the more general splice donor consensus sequence MAGGURR described herein. It is well known in the art that a splice donor may deviate from this consensus, especially in viral genomes where other constraints bear on the same sequence, such as secondary structure for example within a vRNA packaging region. Non-canonical splice sites are also well known in the art, albeit they occur rarely compared to the canonical splice donor consensus sequence.

By “major splice donor site” is meant the first (dominant) splice donor site in the viral vector genome, encoded and embedded within the native viral RNA packaging sequence typically located in the 5′ region of the viral vector nucleotide sequence.

In one aspect the lentiviral vector genome does not contain an active major splice donor site, that is splicing does not occur from the major splice donor site in said lentiviral vector genome, and splicing activity from the major splice donor site is ablated.

The major splice donor site is located in the 5′ packaging region of a lentiviral genome.

In the case of the HIV-1 virus, the major splice donor consensus sequence is (for DNA) TG/GTRAGT (where A is adenosine, T is thymine, G is guanine, C is cytosine, R is a purine and “/” indicates the cleavage site).

In one aspect of the invention, the major splice donor site may have the following consensus sequence, wherein R is a purine and “/” is the cleavage site:

(SEQ ID NO: 16)
TG/GTRAGT

In one aspect, R may be guanine (G).

In one aspect of the invention, the major splice donor and cryptic splice donor region may have the following core sequence, wherein “/” are the cleavage sites at the major splice donor and cryptic splice donor sites:

(SEQ ID NO: 17)
/GTGA/GTA.

In one aspect of the invention, the MSD-mutated vector genome may have at least two mutations in the major splice donor and cryptic splice donor ‘region’ (SEQ ID NO: 17), wherein the first and second ‘GT’ nucleotides are the immediately 3′ of the major splice donor and cryptic splice donor nucleotides respectively

In one aspect of the invention the major splice donor consensus sequence is CTGGT (SEQ ID NO: 18). The major splice donor site may contain the sequence CTGGT.

In one aspect the lentiviral vector genome, prior to inactivation of the splice sites, comprises the sequence as set forth in any of SEQ ID NOs: 16, 17, 18 and/or 19.

In one aspect the lentiviral vector genome comprises an inactivated major splice donor site which would otherwise have a cleavage site between nucleotides corresponding to nucleotides 2 and 3 of SEQ ID NO: 16.

According to the invention as described herein, the lentiviral vector genome may also contain an inactive cryptic splice donor site. In one aspect the lentiviral vector genome does not contain an active cryptic splice donor site adjacent to (3′ of) the major splice donor site, that is to say that splicing does not occur from the adjacent cryptic splice donor site, and splicing from the cryptic splice donor site is ablated.

The term “cryptic splice donor site” refers to a nucleic acid sequence which does not normally function as a splice donor site or is utilised less efficiently as a splice donor site due to the adjacent sequence context (e.g. the presence of a nearby ‘preferred’ splice donor), but can be activated to become a more efficient functioning splice donor site by mutation of the adjacent sequence (e.g. mutation of the nearby ‘preferred’ splice donor).

In one aspect, the cryptic splice donor site is the first cryptic splice donor site 3′ of the major splice donor.

In one aspect the cryptic splice donor site is within 6 nucleotides of the major splice donor site on the 3′ side of the major splice donor site. Preferably the cryptic splice donor site is within 4 or 5, preferably 4, nucleotides of the major splice donor cleavage site.

In one aspect of the invention, the cryptic splice donor site has the consensus sequence TGAGT (SEQ ID NO: 19).

In one aspect, the lentiviral vector genome comprises an inactivated cryptic splice donor site which would otherwise have a cleavage site between nucleotides corresponding to nucleotides 6 and 7 of SEQ ID NO: 16.

In one aspect of the invention, the major splice donor site and/or adjacent cryptic splice donor site contain a “GT” motif. In one aspect of the invention, both the major splice donor site and adjacent cryptic splice donor site contain a “GT” motif which is mutated. The mutated GT motifs may inactivate splice activity from both the major splice donor site and adjacent cryptic splice donor site.

A variety of different types of mutations can be introduced into the lentiviral vector genome in order to inactivate the major and adjacent cryptic splice donor sites.

In one aspect the mutation is a functional mutation to ablate or suppress splicing activity in the splice region. The lentiviral vector genome as described herein may contain a mutation or deletion in any of the nucleotides in any of SEQ ID NOs: 16, 17, 18 and/or 19. Suitable mutations will be known to one skilled in the art, and are described herein.

For example, a point mutation can be introduced into the nucleic acid sequence. The term “point mutation,” as used herein, refers to any change to a single nucleotide. Point mutations include, for example, deletions, transitions, and transversions; these can be classified as nonsense mutations, missense mutations, or silent mutations when present within protein coding sequence. A “nonsense” mutation produces a stop codon. A “missense” mutation produces a codon that encodes a different amino acid. A “silent” mutation produces a codon that encodes either the same amino acid or a different amino acid that does not alter the function of the protein. One or more point mutations can be introduced into the lentiviral vector genome comprising the cryptic splice donor site. For example, the lentiviral vector genome comprising the major and/or cryptic splice site can be mutated by introducing two or more point mutations therein.

At least two point mutations can be introduced in several locations within the nucleic acid sequence comprising the major splice donor and cryptic splice donor sites to achieve attenuation of splicing from the splice donor region. In one aspect, the mutations may be within the four nucleotides at the splice donor cleavage site; in the canonical splice donor consensus sequence this is A¹G²/G³T⁴, wherein “/” is the cleavage site. It is well known in the art that a splice donor cleavage site may deviate from this consensus, especially in viral genomes where other constraints bear on the same sequence, such as secondary structure for example within a vRNA packaging region. It is well known that the G³T⁴ dinucleotide is generally the least variable sequence within the canonical splice donor consensus sequence, and mutations to the G³ and or T⁴ will most likely achieve the greatest attenuating effect. For example, for the major splice donor site in HIV-1 viral vector genomes this can be T¹G²/G³T⁴, wherein “/” is the cleavage site. For example, for the cryptic splice donor site in HIV-1 viral vector genomes this can be G¹A²/G³T⁴, wherein “/” is the cleavage site. Additionally, the point mutation(s) can be introduced adjacent to a splice donor site. For example, the point mutation can be introduced upstream or downstream of a splice donor site. In embodiments where the lentiviral vector genome comprising a major and/or cryptic splice donor site is mutated by introducing multiple point mutations therein, the point mutations can be introduced upstream and/or downstream of the cryptic splice donor site.

In one embodiment, the major splice donor site and/or cryptic splice donor site are deleted.

Construction of Splice Site Mutants

Splice site mutants of the present invention may be constructed using a variety of techniques. For example, mutations may be introduced at particular loci by synthesising oligonucleotides containing a mutant sequence, flanked by restriction sites enabling ligation to fragments of the native sequence. Following ligation, the resulting reconstructed sequence comprises a derivative having the desired nucleotide insertion, substitution, or deletion.

Other known techniques allowing alterations of DNA sequence include recombination approaches such as Gibson assembly, Golden-gate cloning and In-fusion.

Alternatively, oligonucleotide-directed site-specific (or segment specific) mutagenesis procedures may be employed to provide an altered sequence according to the substitution, deletion, or insertion required. Deletion or truncation derivatives of splice site mutants may also be constructed by utilising convenient restriction endonuclease sites adjacent to the desired deletion.

Subsequent to restriction, overhangs may be filled in, and the DNA religated.

Exemplary methods of making the alterations set forth above are disclosed by Sambrook et al. (Molecular cloning: A Laboratory Manual, 2d Ed., Cold Spring Harbor Laboratory Press, 1989).

Splice site mutants may also be constructed utilising techniques of PCR mutagenesis, chemical mutagenesis, chemical mutagenesis (Drinkwater and Klinedinst, 1986) by forced nucleotide misincorporation (e.g., Liao and Wse, 1990), or by use of randomly mutagenised oligonucleotides (Horwitz et al., 1989).

Combination with Modified U1 to Improve Vector Titre

The output titres of lentiviral vectors can be enhanced by co-expressing non-coding RNAs based on U1 snRNAs, which have been modified so that they no longer target the endogenous sequence (a splice donor site) but now target a sequence within the vRNA molecule. MSD-mutated, 3^{rd} generation (i.e. U3/tat-independent) LVs can be produced to high titre by co-expression of a modified U1 snRNA directed to bind to the 5′packaging region of the vector genome RNA during production.

Vector genomes harbouring a broad range of mutation types within the major splice donor region (point mutations, region deletion, and sequence replacement) that lead to reduced titres may be used in combination with a modified U1 snRNA. The approach may comprise co-expression of modified U1 snRNAs together with the other vector components during vector production. The modified U1 snRNAs are designed such that binding to the consensus splice donor site has been ablated by replacing it with a heterologous sequence that is complementary to a target sequence within the vector genome vRNA.

In one aspect of the invention as described herein, the lentiviral vector genome may be used in combination with a modified U1 snRNA.

The elements within a pre-mRNA that are required for splicing include the 5′ splice donor signal, the sequence surrounding the branch point and the 3′ splice acceptor signal. Interacting with these three elements is the spliceosome, which is formed by five small nuclear RNAs (snRNAs), including U1 snRNA, and associated nuclear proteins (snRNP). U1 snRNA is expressed by a polymerase II promoter and is present in most eukaryotic cells (Lund et al., 1984, J. Biol. Chem., 259:2013-2021). U1 snRNA contains a short sequence at its 5′-end that is broadly complementary to the 5′ splice donor sites at exon-intron junctions. U1 snRNA participates in splice-site selection and spliceosome assembly by base pairing to the 5′ splice donor site.

Human U1 snRNA (small nuclear RNA) is 164 nt long with a well-defined structure consisting of four stem—loops (see FIG. 8). The endogenous non-coding RNA, U1 snRNA, binds to the consensus 5′ splice donor site (e.g. 5′-MAGGURR-3′, wherein M is A or C and R is A or G) via the native splice donor annealing sequence (e.g. 5′-ACUUACCUG-3′) during early steps of intron splicing. As defined herein the modified U1 snRNA for use according to the present invention is modified to introduce a heterologous sequence that is complementary to a target sequence within the vector genome vRNA molecule at the site of the native splice donor targeting/annealing sequence (see FIG. 8).

As used herein, the terms “modified U1 snRNA”, “re-directed U1 snRNA”, “re-targeted U1 snRNA”, “re-purposed U1 snRNA” and “mutant U1 snRNA”, mean a U1 snRNA that has been modified so that it is no longer complementary to the consensus 5′ splice donor site sequence (e.g. 5′-MAGGURR-3′) that it uses to initiate the splicing process of a target gene. Thus, a modified U1 snRNA is a U1 snRNA which has been modified so that it is no longer complementary to the splice donor site sequence (e.g. 5′-MAGGURR-3′). Instead, the modified U1 snRNA is designed so that it targets or is complementary to a nucleotide sequence having a unique RNA sequence within the packaging region of a MSD-mutated lentiviral vector genome molecule (target site), i.e. a sequence that is unrelated to splicing of the vRNA.

As used herein, the terms “native splice donor annealing sequence” and “native splice donor targeting sequence” mean the short sequence at the 5′-end of the endogenous U1 snRNA that is broadly complementary to the consensus 5′ splice donor site of introns. The native splice donor annealing sequence may be 5′-ACUUACCUG-3′.

As used herein, the term “consensus 5′ splice donor site” means the consensus RNA sequence at the 5′ end of introns used in splice-site selection, e.g. having the sequence 5′-MAGGURR-3′.

As used herein, the terms “nucleotide sequence within the packaging region of a MSD-mutated lentiviral vector genome sequence”, “target sequence” and “target site” mean a site having a particular RNA sequence within the packaging region of a MSD-mutated lentiviral vector genome molecule which has been preselected as the target site for binding/annealing the modified U1 snRNA.

As used herein, the terms “packaging region of a MSD-mutated lentiviral vector genome molecule” and “packaging region of an MSD-mutated lentiviral vector genome sequence” means the region at the 5′ end of an MSD-mutated lentiviral vector genome from the beginning of the 5′ U5 domain to the terminus of the sequence derived from gag gene. Thus, the packaging region of a MSD-mutated lentiviral vector genome molecule includes the 5′ U5 domain, PBS element, stem loop (SL) 1 element, SL2 element, SL3ψ element, SL4 element and the sequence derived from the gag gene. It will be understood by the person skilled in the art that, if the complete gag gene is to be provided in trans during lentiviral vector production, the term “packaging region of a lentiviral vector genome molecule” may mean the region at the 5′ end of the MSD-mutated lentiviral vector genome molecule from the beginning of the 5′ U5 domain through to the ‘core’ packaging signal at the SL3 ψ element, and the native gag nucleotide sequence from the ATG codon (present within SL4) to the end of the remaining gag nucleotide sequence present on the vector genome.

As used herein, the term “sequence derived from gag gene” means, any native sequence of the gag gene derived from the ATG codon to nucleotide 688 (Kharytonchyk, S. et. al., 2018, J. Mol. Biol., 430:2066-79) that may be present, e.g. remain, in the vector genome.

As used herein, the terms “to introduce within the native splice donor annealing sequence a heterologous sequence” and “to introduce within the native splice donor annealing sequence at the 5′ end of the U1 snRNA a heterologous sequence” include to replace the native splice donor annealing sequence all or in part with said heterologous sequence or to modify the native splice donor annealing sequence to have the same sequence as said heterologous sequence.

As used herein, the term “enhances lentiviral vector titres” includes “increases lentiviral vector titres”, “recovers lentiviral vector titres” and “improves lentiviral vector titres”.

Accordingly, in one embodiment, the modified U1 snRNA has been modified to bind to a nucleotide sequence within the packaging region of an MSD-mutated lentiviral vector genome sequence.

In some embodiments, the modified U1 snRNA is modified at the 5′ end relative to the endogenous U1 snRNA to introduce a heterologous sequence that is complementary to a nucleotide sequence within the packaging region of an MSD-mutated lentiviral vector genome.

In some embodiments, the modified U1 snRNA is modified at the 5′ end relative to the endogenous U1 snRNA to introduce within the native splice donor annealing sequence a heterologous sequence that is complementary to a nucleotide sequence within the packaging region of an MSD-mutated lentiviral vector genome.

The modified U1 snRNA may be modified at the 5′ end relative to the endogenous U1 snRNA to replace a sequence encompassing the native splice donor annealing sequence with a heterologous sequence that is complementary to said nucleotide sequence.

The modified U1 snRNA may be a modified U1 snRNA variant. The U1 snRNA variant which is modified in accordance with the invention may be a naturally occurring U1 snRNA variant, a U1 snRNA variant containing a mutation within the stem loop I region ablating U1-70K protein binding, or a U1 snRNA variant containing a mutation in the stem loop II region ablating U1A protein binding.

In some embodiments, the modified U1 snRNA as described herein comprises a nucleotide sequence having at least 70% identity (suitable at least 75%, at least 80%, 85%, 90%, 91%, 92%, 93%, 94%, 95%, 96%, 97%, 98%, 99% identity) with the main U1 snRNA sequence [clover leaf] (nt 410-562) of the U1_256 sequence as described herein. The U1_256 sequence is as follows:

(SEQ ID NO: 20)
TAAGGACCAGCTTCTTTGGGAGAGAACAGACGCAG
GGGCGGGAGGGAAAAAGGGAGAGGCAGACGTCACT
TCCCCTTGGCGGCTCTGGCAGCAGATTGGTCGGTT
GAGTGGCAGAAAGGCAGACGGGGACTGGGCAAGGC
ACTGTCGGTGACATCACGGACAGGGCGACTTCTAT
GTAGATGAGGCAGCGCAGAGGCTGCTGCTTCGCCA
CTTGCTGCTTCACCACGAAGGAGTTCCCGTGCCCT
GGGAGCGGGTTCAGGACCGCTGATCGGAAGTGAGA
ATCCCAGCTGTGTGTCAGGGCTGGAAAGGGCTCGG
GAGTGCGCGGGGCAAGTGACCGTGTGTGTAAAGAG
TGAGGCGTATGAGGCTGTGTCGGGGCAGAGGCCCA
AGATCTCatttgccgtgcgcgcttGCAGGGGAGAT
ACCATGATCACGAAGGTGGTTTTCCCAGGGCGAGG
CTTATCCATTGCACTCCGGATGTGCTGACCCCTGC
GATTTCCCCAAATGTGGGAAACTCGACTGCATAAT
TTGTGGTAGTGGGGGACTGCGTTCGCGCTTTCCCC
TGGTTTCAAAAGTAGACTGTACGCTAAGGGTCATA
TCTTTTTTTGTTTTGGTTTGTGTCTTGGTTGGCGT
CTTAAATGTTAA
Key:
Upper case only = U1 Polll promoter (nt1-392);
lower case = retargeting region (nt393-409);
lower case bold = retargeting sequence
[in this example targeting nt256-270 of wild
type HIV-1 packaging signal] (nt395-409);
upper case italics = main U1 snRNA sequence
[clover-leaf] (nt410-562);
upper case underlined = transcription
termination region (nt563-652).

In some embodiments, the modified U1 snRNA comprises the main U1 snRNA sequence [clover leaf] (nt 410-562) of the U1_256 sequence as described herein. The main U1 snRNA sequence [clover leaf] (nt 410-562) of the U1_256 sequence (SEQ ID NO: 20) is as follows:

(SEQ ID NO: 21)
GCAGGGGAGATACCATGATCACGAAGGTGGTTTTCCCAGGGCGA
GGCTTATCCATTGCACTCCGGATGTGCTGACCCCTGCGATTTCC
CCAAATGTGGGAAACTCGACTGCATAATTTGTGGTAGTGGGGGA
CTGCGTTCGCGCTTTCCCCTG.

In some embodiments, the modified U1 snRNA comprises a target-annealing sequence as shown in Table 1.

TABLE 1
A list of sequences describing the target-
annealing sequences (heterologous
sequence that is complementary to the
target sequence) within test modified U1
snRNAs and control U1 snRNAs. Nucleotides
are presented as DNA as they would be encoded
within their respective expression cassettes
at the ‘retargeting region’. The (AT) motif
was present in all initial constructs, which
forms the first two nucleotides of the U1 snRNA
molecule in each case. The target sequence
numbers refer to targets in the NL4-3
(GenBank: M19921.2) or HXB2 (GenBank: K03455.1)
strains of HIV-1 where denoted

| | | U1 snRNA |
|---|---|---|
| Modified | | target- |
| U1 | HIV-1 target | annealing |
| snRNA* | sequence [NL4-3]** | sequence |
| U1_16 | 16-GACCAGATCTGAGCC-30 | (AT)GGCT |
| | | CAGATCTG |
| | | GTC |
| U1_31 | 31-TGGGAGCTCTCTGGC-45 | (AT)GCCA |
| | | GAGAGCTC |
| | | CCA |
| U1_76 | 76-TAAAGCTTGCCTTGA-90 | (AT)TCAA |
| | | GGCAAGCT |
| | | TTA |
| U1_136 | 136-TAGAGATCCCTGAGA-150 | (AT)TCTG |
| | | AGGGATCT |
| | | CTA |
| U1_179 | 179-GCAGTGGCG-187 | (AT)CGC |
| (9 nt) | | CACTGC |
| U1_181 | 181-AGTGGCGCCCGAACA-195 | (AT)TGTT |
| | | CGGGCGCC |
| | | ACT |
| U1_196 | 196-GGGACTTGAAAGCGA-210 | (AT)TCGC |
| | | TTTCAAGT |
| | | CCC |
| U1_211 | 211-AAGggAAaCCAGAGG-225 | (AT)CCTC |
| | | TGGTTTCC |
| | | CTT |
| U1_226 | 226-AGcTCTCTCGACGCA-240 | (AT)TGCG |
| | | TCGAGAGA |
| | | GCT |
| U1_241 | 24l-GGACTCGGCTTGCTG-255 | (AT)CAGC |
| | | AAGCCGAG |
| | | TCC |
| U1_256 | 256-AAGCGCGCACGGCAA-270 | (AT)TTGC |
| | | CGTGCGCG |
| | | CTT |
| U1_271 | 271-GAGGCGAGGGGCGGC-285 | (AT)GCCG |
| | | CCCCTCGC |
| | | CTC |
| U1_286 | 286-GACTGGTGAGTACGC-300 | (AT)GCGT |
| | | ACTCACCA |
| | | GTC |
| U1_305 | 305-AATTTTGAC(TA)-313/5 | (AT)GTCA |
| (9 nt) | | AAATT |
| U1_305 | 305-AATTTTGACTAGCGG-319 | (AT)CCGC |
| | | TAGTCAAA |
| | | ATT |
| U1_316 | 316-GCGGAGGCTAGAAGG-330 | (AT)CCTT |
| | | CTAGCCTC |
| | | CGC |
| U1_331 | 331-AGAGAGATGGGTGCG-345 | (AT)CGCA |
| | | CCCATCTC |
| | | TCT |
| U1_346 | 346-AGAGCGTCgGTATTA-360 | (AT)TAAT |
| | | ACTGACGC |
| | | TCT |
| U1_361 | 361-AGCGGGGGAGAATTA-375 | (AT)TAAT |
| | | TCTCCCCC |
| | | GCT |
| U1_376 | 376-GATCGCGATGGGAAA-390 | (AT)TTTC |
| | | CCATCGCG |
| | | ATC |
| U1_391 | 389-AAATTCGGTTAAGGC-403 | (AT)GCCT |
| | | TAACCGAA |
| | | TTT |
| U1_690 | 7159-GATCTTCAGACCTGG-7173 | (AT)CCAG |
| | | GTCTGAAG |
| | | ATC |
| U1_1203 | 7672-TTACACAAGCTTAAT-7686 | (AT)ATTA |
| | | AGCTTGTG |
| | | TAA |
| U1_1546 | 4375-TAGTAGAGATAATAG-4389 | (AT)CTAT |
| | | TATGTCTA |
| | | CTA |

*numbering relative to vector genome RNA sequence
**lower case target sequence is for (HXB2), underlined target sequence is an AA > CGCG frameshift in the gag ORF (U1 376)

In some embodiments, the native splice donor annealing sequence, may be all or in part replaced with a heterologous sequence that is complementary to a nucleotide sequence within the packaging region of an MSD-mutated lentiviral vector genome. Suitably, 1-11 (suitably 2-11, 3-11, 5-11, 1, 2, 3, 4, 5, 6, 7, 8, 9, 10 or 11), nucleic acids of the native splice donor annealing sequence are replaced with a heterologous sequence that is complementary to a nucleotide sequence within the packaging region of an MSD-mutated lentiviral vector genome. In a preferred embodiment, the entire native splice donor annealing sequence is replaced with a heterologous sequence that is complementary to a nucleotide sequence within the packaging region of an MSD-mutated lentiviral vector genome, i.e. the native splice donor annealing sequence (e.g. 5′-ACUUACCUG-3′) is fully replaced with a heterologous sequence as described herein.

In some embodiments, a heterologous sequence that is complementary to a nucleotide sequence within the packaging region of an MSD-mutated lentiviral vector genome comprises at least 7, at least 9 or at least 15 nucleotides of complementarity to said nucleotide sequence. Suitably, a heterologous sequence for use in the present invention may comprise 7-25 (suitably 7-20, 7-15, 9-15, 7, 8, 9, 10, 11, 12, 13, 14, 15, 16, 17, 18, 19, 20, 21, 22, 23, 24 or 25) nucleotides.

The modified U1 snRNAs as described herein may be designed by (a) selecting a target site in the packaging region of an MSD-mutated lentiviral vector genome for binding the modified U1 snRNA (the preselected nucleotide site); and (b) introducing within the native splice donor annealing sequence (e.g. 5′-ACUUACCUG-3′) at the 5′ end of the U1 snRNA a heterologous sequence that is complementary to the preselected nucleotide site selected in step (a).

The introduction of a heterologous sequence that is complementary to the target site within, or in place of, the native splice donor annealing sequence (e.g. 5′-ACUUACCUG-3′) at the 5′ end of the endogenous U1 snRNA using conventional techniques in molecular biology is within the capabilities of a person of ordinary skill in the art. The modification of the native splice donor annealing sequence (e.g. 5′-ACUUACCUG-3′) at the 5′ end of the endogenous U1 snRNA to have the same sequence as a heterologous sequence that is complementary to the target site using conventional techniques in molecular biology is within the capabilities of a person of ordinary skill in the art. For example, suitable methods include directed mutagenesis or random mutagenesis followed by selection for mutations which provide a modified U1 snRNA as described herein.

The modified U1 snRNAs as described herein can be manufactured according to methods generally known in the art. For example, the modified U1 snRNAs can be manufactured by chemical synthesis or recombinant DNA/RNA technology.

In one aspect the nucleotide sequence encoding a modified U1 snRNA may be on a different nucleotide sequence, for example on a different plasmid.

TRIP System

WO2015/092440 (incorporated herein by reference) discloses the use of a heterologous translation control system in eukaryotic cell cultures to repress the translation of the NOI (repress transgene expression) during viral vector production and thus repress or prevent expression of the protein encoded by the NOI. This system is referred to as the Transgene Repression In vector Production cell system or TRIP system. In one form, the TRIP system utilises the bacterial trp operon regulation protein, tryptophan RNA-binding attenuation protein (TRAP), and the TRAP binding site/sequence (tbs) to mediate transgene repression.

The use of this system does not impede the production of packageable vector genome molecules nor the activity of vector virions, and does not interfere with the long-term expression of the NOI in the target cell.

In one aspect, the lentiviral vector genome comprises a tbs.

In some embodiments of the present invention, the nucleotide of interest is operably linked to the tbs. In some embodiments, the nucleotide of interest is translated in a target cell which lacks TRAP.

The tbs may be capable of interacting with TRAP such that translation of the nucleotide of interest is repressed or prevented in a viral vector production cell.

Tryptophan RNA-Binding Attenuation Protein (TRAP)

Tryptophan RNA-binding attenuation protein (TRAP) is a bacterial protein that has been extensively characterised in Bacillus subtilis. It regulates tryptophan biosynthesis directed from the trpEDCFBA operon by participating in either transcription attenuation or translational control mechanisms (reviewed in Gollnick, B., Antson, and Yanofsky (2005) Annual Review of Genetics 39: 47-68).

In its natural context TRAP regulates tryptophan biosynthesis and transport by three distinct mechanisms:
1. Attenuation of transcription of the trpEDCFBA operon (Shimotsu H, K. M., Yanofsky C, Henner D J. (1986) Journal of Bacteriology 166: 461-471).
2. Promotion of formation of the trpE and trpD Shine-Dalgarno blocking hairpin (Yakhnin H, B. J., Yakhnin A V, Babitzke P. (2001) Journal of Bacteriology 183(20): 5918-5926).
3. Blocking ribosome access to the trpG and yhaG ribosome binding sites (Yang M, d. S. A., van Loon APGM, Gollnick P. (1995) Journal of Bacteriology 177: 4272-4278).

In Bacillus subtilis TRAP is encoded by a single gene (mtrB) and the functional protein is composed of 11 identical subunits arranged as a toroid ring (Antson AA, D. E., Dodson G, Greaves R B, Chen X, Gollnick P. (1999) Nature 401(6750): 235-242). It is activated to interact with RNA by binding up to 11 molecules of tryptophan in pockets between neighbouring subunits. The target RNA is wound around the outside of this quaternary ring structure (Babitzke P, S. J., Shire S J, Yanofsky C. (1994) Journal of Biological Chemistry 269: 16597-16604).

The TRAP open-reading frame may be codon-optimised for expression in mammalian (e.g. Homo sapiens) cells, since the bacterial gene sequence is likely to be non-optimal for expression in mammalian cells. The sequence may also be optimised by removing potential unstable sequences and splicing sites. The use of a HIS-tag C-terminally expressed on the TRAP protein appears to offer a benefit in terms of translation repression and may optionally be used. This C-terminal HIS-tag may improve solubility or stability of the TRAP within eukaryotic cells, although an improved functional benefit cannot be excluded. Nevertheless, both HIS-tagged and untagged TRAP allowed robust repression of transgene expression. Certain cis-acting sequences within the TRAP transcription unit may also be optimised; for example, EF1a promoter-driven constructs enable better repression with low inputs of TRAP plasmid compared to CMV promoter-driven constructs in the context of transient transfection.

In one embodiment, the TRAP is derived from a bacteria.

In one embodiment of the present invention, TRAP is derived from a Bacillus species, for example Bacillus subtilis.

In one embodiment, TRAP is derived from the group consisting of: Bacillus subtilis, Aminomonas paucivorans, Desulfotomaculum hydrothermale, B. stearothermophilus, B. stearothermophilus S72N, B. halodurans and Carboxydothermus hydrogenoformans.

In one embodiment, TRAP is encoded by the tryptophan RNA-binding attenuation protein gene family mtrB (TrpBP superfamily e.g. with NCBI conserved domain database #c103437).

In preferred embodiments, the TRAP is C-terminally tagged with six histidine amino acids (HIS×6 tag).

TRAP Binding Site

The term “binding site” is to be understood as a nucleic acid sequence that is capable of interacting with a certain protein.

A consensus TRAP binding site sequence that is capable of binding TRAP is [KAGNN] repeated multiple times (e.g. 6, 7, 8, 9, 10, 11, 12 or more times); such sequence is found in the native trp operon. In the native context, occasionally AAGNN is tolerated and occasionally additional “spacing” N nucleotides result in a functional sequence. In vitro experiments have demonstrated that at least 6 or more consensus repeats are required for TRAP-RNA binding (Babitzke P, Y. J., Campanelli D. (1996) Journal of Bacteriology 178(17): 5159-5163). Therefore, preferably in one embodiment there are 6 or more continuous [KAGN_{≥2}] sequences present within the tbs, wherein K may be T or G in DNA and U or G in RNA.

For TRAP as the RNA-binding protein, preferably the TRIP system works maximally with a tbs sequence containing at least 8 KAGNN repeats, although 7 repeats may be used to still obtain robust transgene repression, and 6 repeats may be used to allow sufficient repression of the transgene to levels that could rescue vector titres. Whilst the KAGNN consensus sequence may be varied to maintain TRAP-mediated repression, preferably the precise sequence chosen may be optimised to ensure high levels of translation in the non-repressed state. For example, the tbs sequences may be optimised by removing splicing sites, unstable sequences or stem-loops that might hamper translation efficiency of the mRNA in the absence of TRAP (i.e. in target cells). Regarding the configuration of the KAGNN repeats of a given tbs, the number of N “spacing” nucleotides between the KAG repeats is preferably two. However, a tbs containing more than two N spacers between at least two KAG repeats may be tolerated (as many as 50% of the repeats containing three Ns may result in a functional tbs as judged by in vitro binding studies; Babitzke P, Y. J., Campanelli D. (1996) Journal of Bacteriology 178(17): 5159-5163). Indeed, it has been shown that an 11× KAGNN tbs sequence can tolerate up to three replacements with KAGNNN repeats and still retain some potentially useful translation-blocking activity in partnership with TRAP-binding.

In one embodiment of the present invention, the TRAP binding site or portion thereof comprises the sequence KAGN_{≥2} (e.g. KAGN₂₋₃). For the avoidance of doubt therefore, this tbs or portion thereof comprises, for example, any of the following repeat sequences: UAGNN, GAGNN, TAGNN, UAGNNN, GAGNNN, or TAGNNN.

“N” is to be understood as specifying any nucleotide at that position in the sequence. For example, this could be G, A, T, C or U. The number of such nucleotides is preferably 2 but up to three, for example 1, 2 or 3, KAG repeats of an 11× repeat tbs or portion thereof may be separated by 3 spacing nucleotides and still retain some TRAP-binding activity that leads to translation repression. Preferably not more than one N₃ spacer will be used in an 11× repeat tbs or portion thereof in order to retain maximal TRAP-binding activity that leads to translation repression.

In another embodiment, the tbs or portion thereof comprises multiple repeats of KAGN_{≥2} (e.g. multiple repeats of KAGN₂₋₃).

In another embodiment, the tbs or portion thereof comprises multiple repeats of the sequence KAGN₂.

In another embodiment, the tbs or portion thereof comprises at least 6 repeats of KAGN_{≥2} (e.g. at least 6 repeats of KAGN₂₋₃).

In another embodiment, the tbs or portion thereof comprises at least 6 repeats of KAGN₂. For example, the tbs or portion thereof may comprise 6, 7, 8, 9, 10, 11, 12 or more repeats of KAGN₂.

In another embodiment, the tbs or portion thereof comprises at least 8 repeats of KAGN_{≥2} (e.g. at least 8 repeats of KAGN₂₋₃).

Preferably, the number of KAGNNN repeats present in the tbs or portion thereof is 1 or less.

In another embodiment, the tbs or portion thereof comprises 11 repeats of KAGN_{≥2} (e.g. 11 repeats of KAGN₂₋₃). Preferably, the number of KAGNNN repeats present in this tbs or portion thereof is 3 or less.

In another embodiment, the tbs or portion thereof comprises 12 repeats of KAGN_{≥2} (e.g. 12 repeats of KAGN₂₋₃).

In a preferred embodiment, the tbs or portion thereof comprises 8-11 repeats of KAGN₂ (e.g. 8, 9, 10 or 11 repeats of KAGN₂).

By “repeats of KAGN_{≥2}” it is to be understood that the general KAGN_{≥2} (e.g. KAGN₂₋₃) motif is repeated. Different KAGN_{≥2} sequences satisfying the criteria of this motif may be joined to make up the tbs or portion thereof. It is not intended that the resulting tbs or portion thereof is limited to repeats of only one sequence that satisfies the requirements of this motif, although this possibility is included in the definition.

An 8-repeat tbs or portion thereof containing one KAGNNN repeat and seven KAGNN repeats retains TRAP-mediated repression activity. Less than 8-repeat tbs sequences or portions thereof (e.g. 7- or 6-repeat tbs sequences or portions thereof) containing one or more KAGNNN repeats may have lower TRAP-mediated repression activity. Accordingly, when fewer than 8-repeats are present, it is preferred that the tbs or portion thereof comprises only KAGNN repeats.

Preferred nucleotides for use in the KAGNN repeat consensus are:
a pyrimidine in at least one of the NN spacer positions;
a pyrimidine at the first of the NN spacer positions;
pyrimidines at both of the NN spacer positions;
G at the K position.

It is also preferred that G is used at the K position when the NN spacer positions are AA (i.e. it is preferred that TAGAA is not used as a repeat in the consensus sequence).

By “capable of interacting” it is to be understood that the nucleic acid binding site (e.g. tbs or portion thereof) is capable of binding to a protein, for example TRAP, under the conditions that are encountered in a cell, for example a eukaryotic viral vector production cell. Such an interaction with an RNA-binding protein such as TRAP results in the repression or prevention of translation of a NOI to which the nucleic acid binding site (e.g. the tbs or portion thereof) is operably linked.

By “operably linked” it is to be understood that the components described are in a relationship permitting them to function in their intended manner. Therefore a tbs or portion thereof for use in the invention operably linked to a NOI is positioned in such a way that translation of the NOI is modified when as TRAP binds to the tbs or portion thereof.

Placement of a tbs or portion thereof capable of interacting with an TRAP upstream of a NOI translation initiation codon of a given open reading frame (ORF) allows specific translation repression of mRNA derived from that ORF. The number of nucleotides separating the tbs or portion thereof and the translation initiation codon may be varied, for example from 0 to 34 nucleotides, without affecting the degree of repression. As a further example, 0 to 13 nucleotides may be used to separate the TRAP-binding site or portion thereof and the translation initiation codon.

The tbs or portion thereof may be placed downstream of an internal ribosome entry site (IRES) to repress translation of the NOI in a multicistronic mRNA. In one embodiment, the lentiviral vector genome comprises a spacer sequence between an IRES and the tbs or the portion thereof. The IRES may be an IRES as described herein under the subheading “Internal ribosome entry site”. The spacer sequence may be between 0 and 30 nucleotides in length, preferably 15 nucleotides in length.

In one embodiment, the spacer sequence between an IRES and the tbs or portion thereof is 3 or 9 nucleotides from the 3′ end of the tbs or portion thereof and the downstream initiation codon of the NOI.

In one embodiment, the tbs or portion thereof lacks a type II restriction enzyme site. In a preferred embodiment, the tbs or portion thereof lacks a Sapl restriction enzyme site.

The practice of the present invention will employ, unless otherwise indicated, conventional techniques of chemistry, molecular biology, microbiology and immunology, which are within the capabilities of a person of ordinary skill in the art. Such techniques are explained in the literature. See, for example, J. Sambrook, E. F. Fritsch, and T. Maniatis (1989) Molecular Cloning: A Laboratory Manual, Second Edition, Books 1-3, Cold Spring Harbor Laboratory Press; Ausubel, F. M. et al. (1995 and periodic supplements) Current Protocols in Molecular Biology, Ch. 9, 13, and 16, John Wiley & Sons, New York, N.Y.; B. Roe, J. Crabtree, and A. Kahn (1996) DNA Isolation and Sequencing: Essential Techniques, John Wiley & Sons; J. M. Polak and James O′D. McGee (1990) In Situ Hybridization: Principles and Practice; Oxford University Press; M. J. Gait (ed.) (1984) Oligonucleotide Synthesis: A Practical Approach, IRL Press; and, D. M. J. Lilley and J. E. Dahlberg (1992) Methods of Enzymology: DNA Structure Part A: Synthesis and Physical Analysis of DNA Methods in Enzymology, Academic Press. Each of these general texts is herein incorporated by reference.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

Methods

Adherent Cell Culture, Transfection and Lentiviral Vector Production

HEK293T.1-65s suspension cells were grown in Freestyle+0.1% CLC (Gibco) at 37° C. in 5% CO₂, in a shaking incubator (25 mm orbit set at 190 RPM). For vector production 1-65s cells in suspension were transferred to an adherent mode by being pelleted by centrifugation and resuspended in an appropriate volume of Dulbecco's Modified Eagle Medium (DMEM) (Sigma) supplemented with 10% heat-inactivated (FBS) (Gibco), 2 mM L-glutamine (Sigma) and 1% non-essential amino acids (NEAA) (Sigma)). The standard scale production of HIV-1 vectors in adherent mode was in 10 cm dishes under the following conditions (all conditions were scaled by area when performed in other formats): 1-65s cells resuspended in complete media were seeded at 3.5×10⁵ cell per mL in 10 mL complete media and approximately 24 hours later the cells were transfected using the following mass ratios of plasmids per 10 cm plate: 4.5 μg Genome, 1.4 μg Gag-Pol, 1.1 μg Rev, 0.7 μg VSV-G and between 0.01 and 2 μg of modified U1 snRNA plasmid.

Transfection was mediated by mixing DNA with Lipofectamine 2000CD in Opti-MEM as per manufacturer's protocol (Life Technologies). Sodium butyrate (Sigma) was added ˜18 hours later to 10 mM final concentration for 5-6 h, before 10 mL fresh serum-free media replaced the transfection media. Typically, vector supernatant was harvested 20-24 hours later, and then filtered (0.22 μm) and frozen at −20/−80° C.

Lentiviral Vector Titration Assays

For lentiviral vector titration by GFP marker-containing cassette, HEK293T cells were seeded at 1.2×10⁴ cells/well in 96-well plates. GFP-encoding viral vectors were used to transduce the cells in complete media containing 8 mg/mL polybrene. The transduced cells were incubated for 2 days at 37° C. in 5% CO₂. Cultures were then prepared for flow cytometry using an Attune-N×T (Thermofisher). Percent GFP expression was measured and vector titres were calculated using a predicted cell count of 1.8×10⁴ cells at the time of transduction (base on typical growth rate), the dilution factor of the vector sample, the percentage positive GFP population and total volume at transduction.

### Example 1: Development of Functional Rev Response Elements with Ablated Sub-ORFs

A key viral cis-acting element retained (and “re-positioned”) within standard lentiviral vectors is the Rev response element (RRE). This RNA element—which fully overlaps with part of the lentivirus envelope ORF—forms a complex RNA structure to which the rev protein binds. In doing so, the rev protein is able to “pull” the vector genome RNA molecule out of the nucleus and make it available for packaging. It is therefore in important feature of the vector genome RNA, and vector titres are substantially reduced in its absence. However, since the RRE is also part of the envelope ORF, the presence of ATG codons within it means there is a potential risk that the RRE-encoded envelope sub-ORFs (and other sub-ORFs in other reading-frames) are translated, should the vector backbone sequence be transcribed in cells (e.g. patient cells) as directed by an upstream cellular promoter.

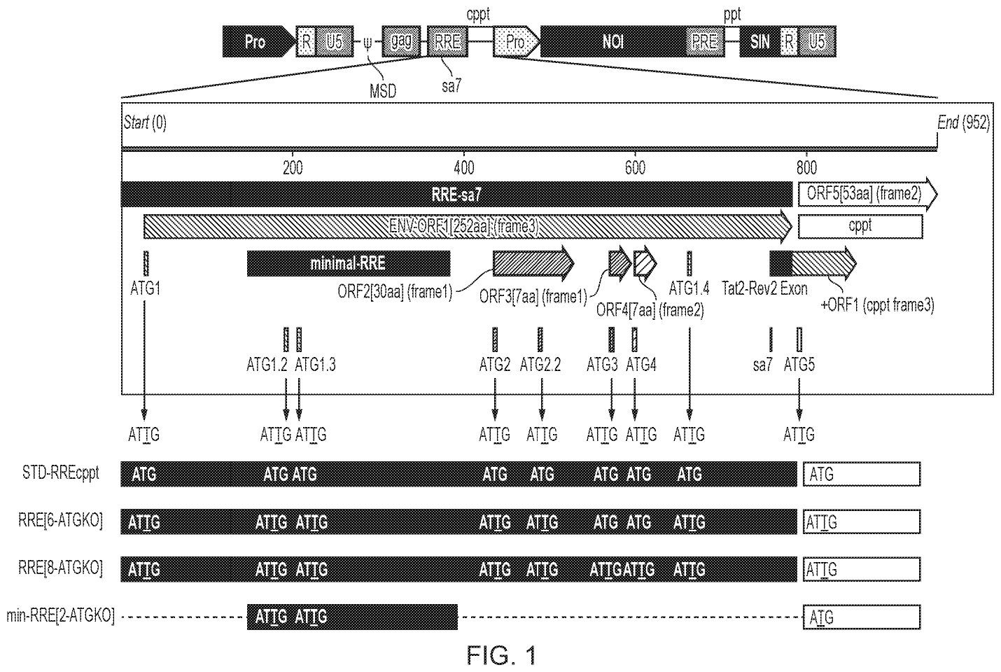

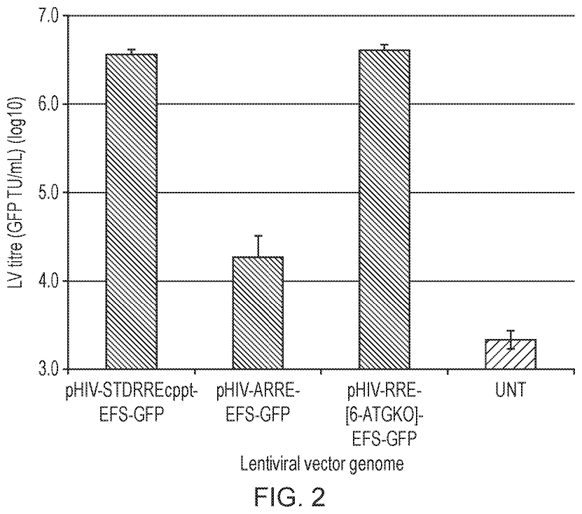

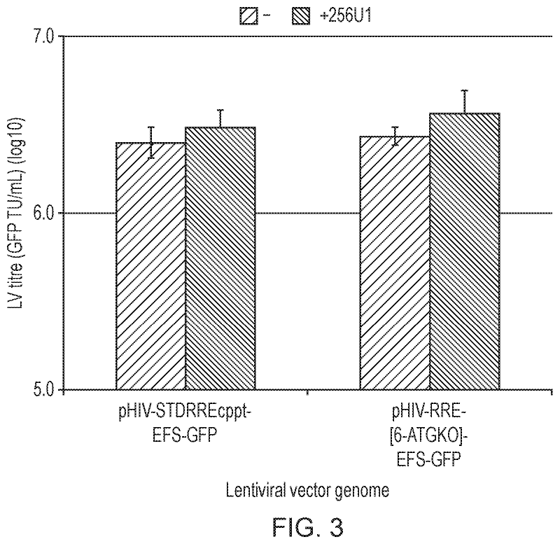

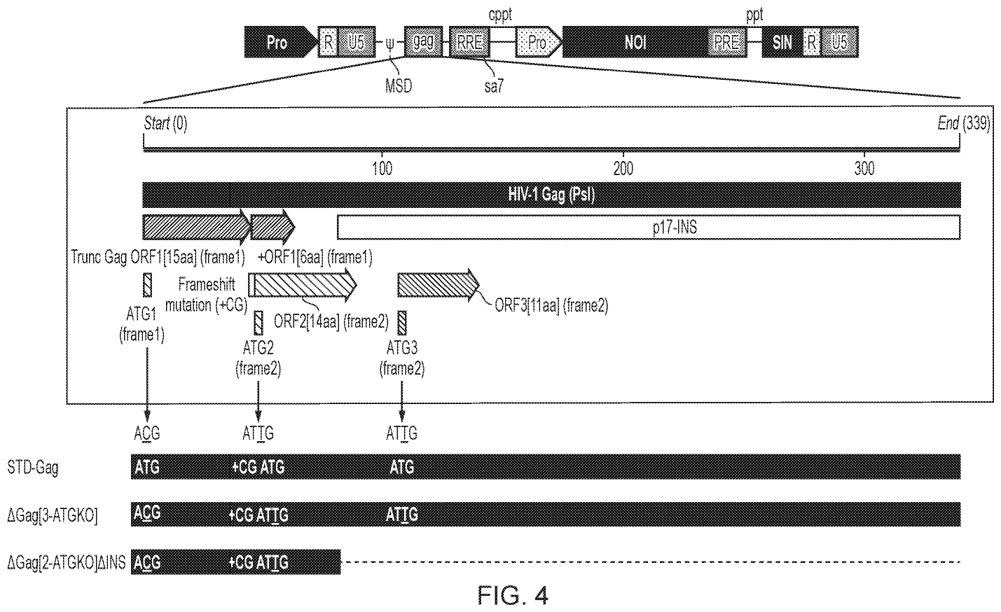

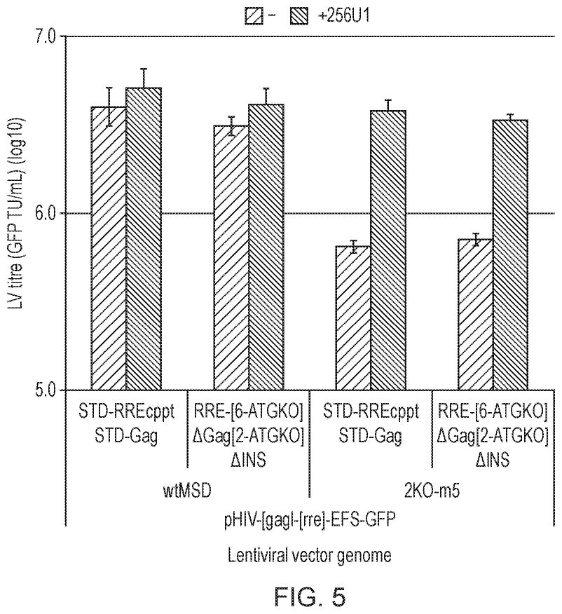

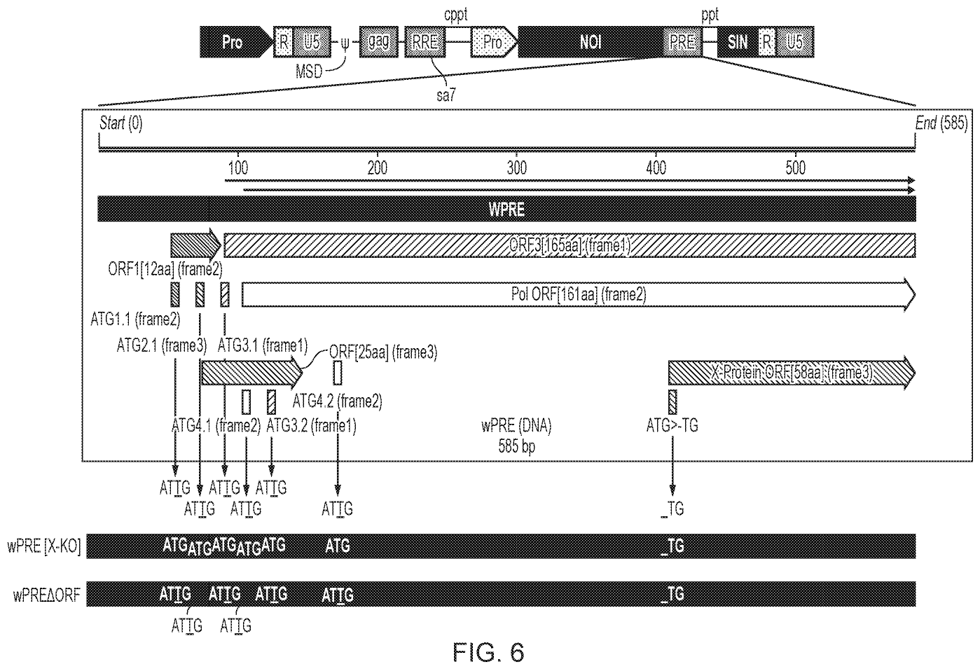

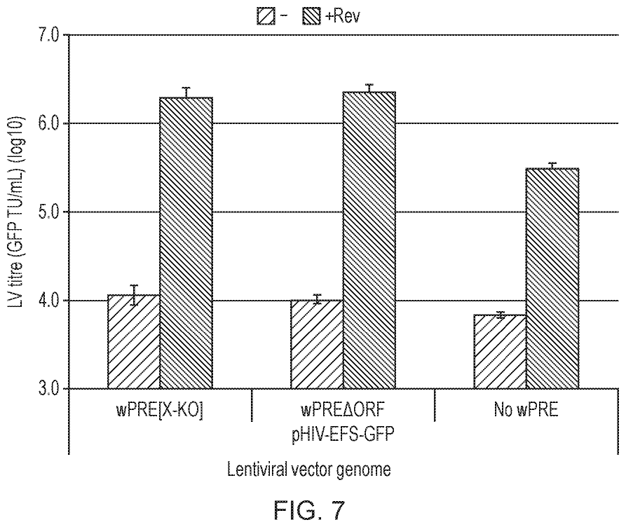

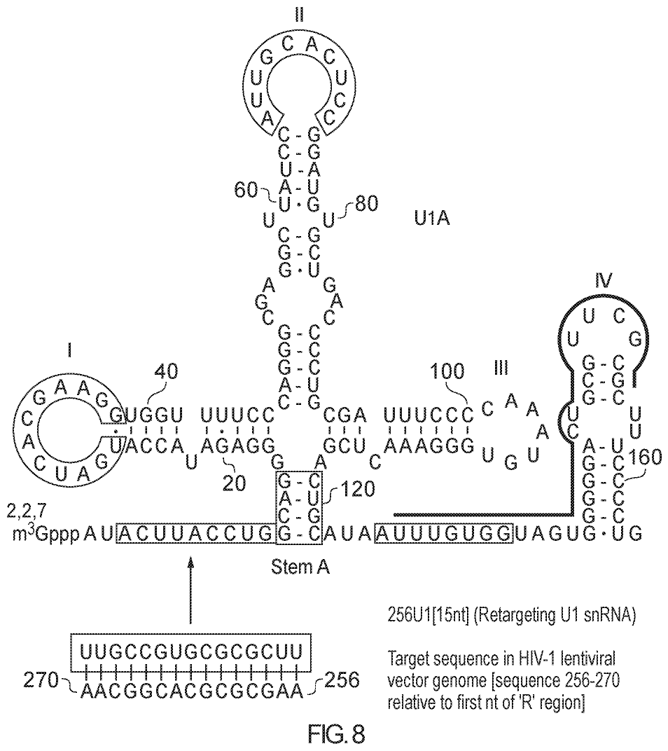

FIG. 1 shows a typical standard, 3^{rd} generation lentiviral vector with the RRE in its typical position, as well as all of the potential ATG translation initiation codons and their associated sub-ORFs (also referred to herein as internal ORFs). Variant RREs wherein 6 or all 8 ATG codons were mutated by single nucleotide insertion were generated, resulting in all sub-ORFs of >7 residues being disrupted. In addition, these were tested with a cppt sequence wherein an upstream ATG codon derived from the Pol gene was also mutated, and the associated sub-ORF disrupted. The variant RRE[6-ATGKO] was cloned into a lentiviral vector expressing GFP, produced in serum-free, suspension HEK293T cells and titrated by flow cytometry of transduced cells (FIG. 2). This variant was compared against the standard RRE-containing vector genome, and a version where the RRE was deleted. The variant was found to be fully active (as evidenced by the equivalent titres compared to the standard vector), despite the array of inserted nucleotides ablating the ATG codons. In addition, the same RRE[6-ATGKO] variant was tested in combination with modified U1 snRNA targeted to the vector genome RNA (which has been demonstrated to increase lentiviral vector titres, probably due to stabilisation of vRNA during production, and behaved similarly to the standard RRE-containing vector (FIG. 3).

### Example 2: Development of Functional Gag Sequences within the Lentiviral Packaging Sequence, with Ablated Sub-ORFs and/or p17-INS Deletions

The Gag sequence retained within the lentiviral vector genome RNA forms part of the (non-core) packaging sequence. Typically, contemporary lentiviral vectors include a mutation within this Gag sequence to mitigate the possibility of translation of the Gag sequence during both vector production (potentially inhibiting correct particle assembly) and in patient cells, should the vector backbone sequence be transcribed by an upstream cellular promoter, representing a safety risk. Typically, a frameshift mutation ˜45nt downstream from the primary ATG Gag codon is inserted but this retains a sub-ORF encoding ˜15 residues of Gag, and several other fused residues derived from the frame-shift. In addition, the Gag sequence typically retains the p17-INS sequence, which has been reported to be important for retaining rev-RRE activity.

FIG. 4 displays a typical standard, 3^{rd} generation lentiviral vector with the Gag sequence in its typical position, as well as all of the potential ATG translation initiation codons and their associated sub-ORFs. Variant Gag sequences were developed harbouring mutations within the ATG codons, thus ablating sub-ORFs. In addition, variant ‘Δgag[2-ATGKO]ΔINS’ was generated, wherein the entire p17-INS sequence was deleted, resulting in a highly minimal Gag sequence. The Δgag[2-ATGKO]ΔINS variant sequence was partnered with the novel RRE variant ‘RRE[6-ATGKO]’ and both cloned into a GFP-expressing lentiviral vector genome containing an intact major splice donor (MSD). In addition, this variant was also cloned into an MSD-mutated lentiviral vector genome, referred to as ‘2KO-m5’. The MSD-mutated vectors do not produce aberrantly spliced RNA during vector production unlike standard, but their titres are reduced. It has been shown that expression of a re-directed U1 snRNA targeted to the vector genome RNA is able to fully rescue this defect. We wanted to test the combination Gag-RRE variant in both the context of MSD-containing and MSD-mutated vector genomes, and their potential rescue by modified U1 snRNA. Serum-free, suspension HEK293T cells were transfected with this novel variant or a standard vector genome construct along with packaging components and +/− modified U1 snRNA, and resulting vector supernatant titrated by flow cytometry of transduced cells (FIG. 5). The results demonstrate that the minimal Gag sequence variant Δgag[2-ATGKO]ΔINS was able to generate comparable vector titres to standard vectors, and was used in combination with the novel RRE variant. The combination variant was also fully responsive to modified U1 snRNA, as titres of the MSD-mutated versions were equally rescued compared to the standard Gag-RRE sequence in the MSD-mutated vector genome. Thus, it was possible to not only ablate all sub-ORFs encoding peptides/proteins of >7 residues (including the sub-ORFs encoding portions of viral proteins), approximately 250nt of additional vector capacity was generated by deletion of the p17-INS sequence.

### Example 3: Development of a Functional wPRE with all Sub-ORFs Fully Ablated

The Woodchuck Hepatitis Virus Postranscription Regulatory Element (wPRE) is a cis-acting RNA sequence that exhibits functionally conserved secondary structure that is essential for function. This element promotes cytoplasmic accumulation of the unspliced RNA of WHV independently of any viral protein. Furthermore, insertion of the wPRE into heterologous mRNAs has been demonstrated to markedly improve gene expression levels. These attributes have led to the inclusion of the wPRE into the 3′UTR of most standard lentiviral vector platforms (third generation). The wPRE element sequences included in lentiviral vectors typically consists of nucleotides 1093-1685 (nucleotide numbering scheme of GenBank Accession No. J04514). This sequence retains a tripartite element required for PRE activity, as well as the promoter and first 60 amino acids of the WHV X-protein. In standard lentiviral vectors, the initiation codon of the X-protein has been ablated by mutation of the start codon to by deletion of ATG to TG in order to improve the safety profile of the vectors. However, additional ATG sequences were left intact within the wPRE that encoded for at least three ORFs. This included a 160 residue ORF derived from the WHV Pol protein that corresponds with the RNAse H domain.

In this example all further ATGs were removed from the wPRE through mutation to ATTG (FIG. 6). This was done in order to further ablate any potential ORFs encoded within the wPRE and therefore improve the safety profile of the lentiviral vector platform. Prior to this work, the consequences of these mutations on wPRE function and vector titres were unknown. The wPRE RNA is highly structured, and at least one stem loop is known to be important for its function as a post-transcriptional regulatory element. However, the full activity of the wPRE seems to require the entire element, suggesting either multiple factor binding sites and/or a higher order structure that has yet to be defined. Therefore, disruption of nucleotide sequences could potentially disrupt the secondary structure of the RNA and hence adversely affect the function of the wPRE. To assess this, 3^{rd} generation lentiviral vectors were produced that either harboured the standard wPRE element with the X-protein ORF ablated (wPRE[X-KO]), a wPRE[X-KO] with all additional ATGs mutated to ATTG (wPREΔORF), or with no wPRE (FIG. 7). These vectors were produced in both the presence and absence of the HIV-1 Rev protein, which promotes cytoplasmic accumulation of unspliced HIV-1 RNA through binding to the RRE element. In the absence of Rev, the titres of all vectors were low, demonstrating the requirement for Rev-RRE mediated export of the vector genomic RNA, regardless of its wPRE status. In the presence of Rev, the titres of vector with no wPRE were over 5-fold lower than those with the standard X-KO wPRE. This demonstrates the functional significance of the wPRE in the lentiviral vector platform. Lentiviral vector with ablation of all additional ATGs in the X-KO wPRE (wPREΔORF) produced similar titres to the standard wPRE X-KO vector. This demonstrates that the mutation of all ATG sequences within the wPRE does not affect its function of improving lentiviral vector titres.

1. A lentiviral vector genome comprising at least one modified viral cis-acting sequence, wherein at least one internal open reading frame (ORF) in the viral cis-acting sequence is disrupted.

2. A lentiviral vector genome according to claim 1, wherein the at least one viral cis-acting sequence is: (a) a Rev response element (RRE); and/or (b) a Woodchuck hepatitis virus (WHV) post-transcriptional regulatory element (WPRE).

3. A lentiviral vector genome comprising a modified Rev response element (RRE), wherein at least one internal open reading frame (ORF) in the RRE is disrupted.

4. A lentiviral vector genome comprising a modified Woodchuck hepatitis virus (WHV) post-transcriptional regulatory element (WPRE), wherein at least one internal open reading frame (ORF) in the WPRE is disrupted.

5. A lentiviral vector genome lacking either (i) a nucleotide sequence encoding Gag-p17 or (ii) a fragment of a nucleotide sequence encoding Gag-p17.

6. The lentiviral vector genome according to claim 5, wherein the lentiviral vector genome does not express Gag-p17 or a fragment thereof.

7. The lentiviral vector genome according to claim 5 or claim 6, wherein the lentiviral vector genome comprises at least one modified viral cis-acting sequence, and wherein at least one internal ORF in the viral cis-acting sequence is disrupted, optionally wherein the viral cis-acting sequence is an RRE and/or a WPRE.

8. The lentiviral vector genome according to any one of claim 1 to 4 or 7, wherein the at least one internal ORF is disrupted by mutating at least one ATG sequence.

9. The lentiviral vector genome according to any one of claim 2, 3, 4, 7 or 8, wherein: (a) the modified RRE comprises less than eight ATG sequences; and/or (b) the modified WPRE comprises less than seven ATG sequences.

10. A lentiviral vector genome comprising a modified Rev response element (RRE), wherein the modified RRE comprises less than eight ATG sequences.

11. A lentiviral vector genome comprising a modified Woodchuck hepatitis virus (WHV) post-transcriptional regulatory element (WPRE), wherein the modified WPRE comprises less than seven ATG sequences.

12. The lentiviral vector genome according to any one of claims 2, 3 or 7 to 10, wherein the RRE is a full-length RRE or a minimal RRE.

13. The lentiviral vector genome according to any one of claims 2, 3, 4 or 7 to 12, wherein:
(i) the RRE comprises:
a) a sequence having at least 80% identity to SEQ ID NO: 1; and/or
b) a sequence having at least 80% identity to SEQ ID NO: 2; and/or
(ii) the WPRE comprises:
a) a sequence having at least 80% identity to SEQ ID NO: 11; and/or
b) a sequence having at least 80% identity to SEQ ID NO: 12.

14. The lentiviral vector genome according to any one of claims 2, 3 or 7 to 13, wherein the modified RRE comprises the sequence as set forth in SEQ ID NO: 1 or SEQ ID NO: 2, or a sequence having at least 80% identity thereto, wherein at least one ATG sequence selected from the group (a)-(h) is mutated:
a) ATG corresponding to positions 27-29 of SEQ ID NO: 2;
b) ATG corresponding to positions 192-194 of SEQ ID NO: 2;
c) ATG corresponding to positions 207-209 of SEQ ID NO: 2;
d) ATG corresponding to positions 436-438 of SEQ ID NO: 2;
e) ATG corresponding to positions 489-491 of SEQ ID NO: 2;
f) ATG corresponding to positions 571-573 of SEQ ID NO: 2;
g) ATG corresponding to positions 599-601 of SEQ ID NO: 2;
h) ATG corresponding to positions 663-665 of SEQ ID NO: 2.

15. The lentiviral vector genome according to any one of claims 2, 3 or 7 to 14, wherein the modified RRE comprises less than seven, less than six, less than five, less than four, less than three, less than two or less than one ATG sequence(s).

16. The lentiviral vector genome according to any one of the preceding claims, wherein the lentiviral vector genome comprises a modified nucleotide sequence encoding gag, and wherein at least one internal ORF in the modified nucleotide sequence encoding gag is disrupted, optionally wherein the at least one internal ORF in the modified nucleotide sequence encoding gag is disrupted by mutating at least one ATG sequence.

17. The lentiviral vector genome according to claim 16, wherein the nucleotide sequence encoding gag comprises a sequence having at least 80% identity to SEQ ID NO: 6 or SEQ ID NO: 7.

18. The lentiviral vector genome according to claim 17, wherein the modified nucleotide sequence encoding gag comprises less than three ATG sequences.

19. The lentiviral vector genome according to any one of claims 2, 3, 4 or 7 to 18, wherein the lentiviral vector genome lacks either (i) a nucleotide sequence encoding Gag-p17 or (ii) a fragment of a nucleotide sequence encoding Gag-p17, optionally wherein the lentiviral vector genome does not express Gag-p17 or a fragment thereof.

20. The lentiviral vector genome according to any one of the preceding claims, wherein the major splice donor site in the lentiviral vector genome is inactivated, optionally further wherein the cryptic splice donor site 3′ to the major splice donor site is inactivated.

21. The lentiviral vector genome according to any one of the preceding claims, wherein the lentiviral vector genome further comprises a nucleotide of interest, optionally wherein the nucleotide of interest gives rise to a therapeutic effect.

22. The lentiviral vector genome according to any one of the preceding claims, wherein the lentiviral vector genome further comprises a tryptophan RNA-binding attenuation protein (TRAP) binding site

23. The lentiviral vector genome according to any one of the preceding claims wherein the lentiviral vector is derived from HIV-1, HIV-2, SIV, FIV, BIV, EIAV, CAEV or Visna lentivirus.

24. A lentiviral vector comprising the lentiviral vector genome of any one of the preceding claims, optionally wherein the lentiviral vector is derived from HIV-1, HIV-2, SIV, FIV, BIV, EIAV, CAEV or Visna lentivirus.

25. A nucleotide sequence encoding the lentiviral vector genome according to any one of claims 1 to 23.

26. An expression cassette comprising the nucleotide sequence according to claim 25.

27. A viral vector production system comprising a set of nucleotide sequences, wherein the nucleotide sequences encode vector components including gag-pol, env, optionally rev, and the lentiviral vector genome according to any one of claims 1 to 23.

28. A cell comprising the lentiviral vector genome according to any one of claims 1 to 23, a nucleotide sequence according to claim 25, the expression cassette according to claim 26 or the viral vector production system according to claim 27.

29. A cell for producing lentiviral vectors comprising:
a) nucleotide sequences encoding vector components including gag-pol and env, and optionally rev, and a nucleotide sequence according to claim 25 or the expression cassette according to claim 26; or
b) the viral vector production system according to claim 27; and
c) optionally a nucleotide sequence encoding a modified U1 snRNA and/or optionally a nucleotide sequence encoding TRAP.

30. A method for producing a lentiviral vector, comprising the steps of:
(i) introducing:
a) nucleotide sequences encoding vector components including gag-pol and env, and optionally rev, and a nucleotide sequence according to claim 25 or the expression cassette according to claim 26; or
b) the viral vector production system according to claim 27; and
c) optionally a nucleotide sequence encoding a modified U1 snRNA and/or optionally a nucleotide sequence encoding TRAP into a cell;
(ii) optionally selecting for a cell which comprises the nucleotide sequences encoding vector components and the lentiviral vector genome; and
(iii) culturing the cell under conditions suitable for the production of the lentiviral vector.

31. A lentiviral vector produced by the method according to claim 30.

32. Use of the lentiviral vector genome according to any one of claims 1 to 23, a nucleotide sequence according to claim 25, the expression cassette according to claim 26, the viral vector production system according to claim 27, or the cell according to claim 28 or claim 29 for producing a lentiviral vector.


SEQUENCE LISTING (70 sequences)

SEQ ID NO: 1 - DNA, 234 bp, Artificial Sequence
  minimal functional RRE (Rev response element)
aggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcct

SEQ ID NO: 2 - DNA, 783 bp, Artificial Sequence
  full-length RRE
tgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggac

SEQ ID NO: 3 - DNA, 236 bp, Artificial Sequence
  modified RRE sequence
aggagctttgttccttgggttcttgggagcagcaggaagcactattgggcgcagcgtcaattgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcct

SEQ ID NO: 4 - DNA, 789 bp, Artificial Sequence
  modified RRE sequence
tgatcttcagacctggaggaggagatattgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactattgggcgcagcgtcaattgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaattgctagttggagtaataaatctctggaacagatttggaatcacacgacctggattggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataattgatagtaggaggcttggtaggtttaagaatagtttttgctgtactttctatagtgaatagagttaggcagggatattcaccattatcgtttcagacccacctcccaaccccgaggggac

SEQ ID NO: 5 - DNA, 791 bp, Artificial Sequence
  modified RRE sequence lacking an ATG sequence
tgatcttcagacctggaggaggagatattgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactattgggcgcagcgtcaattgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaattgctagttggagtaataaatctctggaacagatttggaatcacacgacctggattggagtgggacagagaaattaacaattacacaagcttaatacactccttaattgaagaatcgcaaaaccagcaagaaaagaattgaacaagaattattggaattagataaattgggcaagtttgtggaattggtttaacataacaaattggctgtggtatataaaattattcataattgatagtaggaggcttggtaggtttaagaatagtttttgctgtactttctatagtgaatagagttaggcagggatattcaccattatcgtttcagacccacctcccaaccccgaggggac

SEQ ID NO: 6 - DNA, 339 bp, Artificial Sequence
  truncated nucleotide sequence encoding a part of gag
atgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagagggagagcaaaacaaaagtaaga

SEQ ID NO: 7 - DNA, 81 bp, Artificial Sequence
  minimal truncated nucleotide sequence encoding a part of gag
atgggtgcgagagcgtcagtattaagcgggggagaattagatcgcgatgggaaaaaattcggttaaggccagggggaaaga

SEQ ID NO: 8 - DNA, 341 bp, Artificial Sequence
  modified truncated nucleotide sequence encoding part of gag
acgggtgcgagagcgtcagtattaagcgggggagaattagatcgcgattgggaaaaaattcggttaaggccagggggaaagaaaaaatataaattaaaacatatagtattgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagagggagagcaaaacaaaagtaaga

SEQ ID NO: 9 - DNA, 82 bp, Artificial Sequence
  modified minimal truncated nucleotide sequence encoding a part of gag
acgggtgcgagagcgtcagtattaagcgggggagaattagatcgcgattgggaaaaaattcggttaaggccagggggaaaga

SEQ ID NO: 10 - DNA, 255 bp, Artificial Sequence
  p17-INS (p17 instability element) sequence
aaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagagggagagcaaaacaaaagta

SEQ ID NO: 11 - DNA, 585 bp, Artificial Sequence
  WPRE (Woodchuck hepatitis virus (WHV) post-transcriptional regulatory element) sequence
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctcc

SEQ ID NO: 12 - DNA, 585 bp, Artificial Sequence
  WPRE sequence  which contains a disrupted X-protein ORF
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctcc

SEQ ID NO: 13 - DNA, 481 bp, Artificial Sequence
  retained Pol ORF sequence
atgctattgcttcccgtatggctttcattttctcctccttgtataaatctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctcc

SEQ ID NO: 14 - DNA, 587 bp, Artificial Sequence
  modified WPRE sequence in which all ATG codons within the retained Pol ORF are mutated
aatcaacctctggattacaaaatttgtgaaagattgactggtattcttaactatgttgctccttttacgctatgtggatacgctgctttaatgcctttgtatcattgctattgcttcccgtatggctttcattttctcctccttgtataaatcctggttgctgtctctttattgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctcc

SEQ ID NO: 15 - DNA, 591 bp, Artificial Sequence
  modified WPRE sequence lacking an ATG sequence
aatcaacctctggattacaaaatttgtgaaagattgactggtattcttaactattgttgctccttttacgctattgtggatacgctgctttaattgcctttgtatcattgctattgcttcccgtattggctttcattttctcctccttgtataaatcctggttgctgtctctttattgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctcc

SEQ ID NO: 16 - DNA, 8 bp, Artificial Sequence
  major splice donor site consensus sequence
tggtragt

SEQ ID NO: 17 - DNA, 7 bp, Artificial Sequence
  major splice donor and cryptic splice donor site
gtgagta

SEQ ID NO: 18 - DNA, 5 bp, Artificial Sequence
  major splice donor consensus sequence
ctggt

SEQ ID NO: 19 - DNA, 5 bp, Artificial Sequence
  cryptic splice donor site consensus sequence
tgagt

SEQ ID NO: 20 - DNA, 642 bp, Artificial Sequence
  U1_256 sequence
taaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgctgcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcccaagatctcatttgccgtgcgcgcttgcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccctggtttcaaaagtagactgtacgctaagggtcatatctttttttgttttggtttgtgtcttggttggcgtcttaaatgttaa

SEQ ID NO: 21 - DNA, 153 bp, Artificial Sequence
  main U1 snRNA sequence [clover leaf] (nt 410-562) of the U1_256 sequence
gcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccctg

SEQ ID NO: 22 - DNA, 15 bp, Artificial Sequence
  HIV-1 target sequence
gaccagatctgagcc

SEQ ID NO: 23 - DNA, 17 bp, Artificial Sequence
  U1 snRNA target-annealing sequence
atggctcagatctggtc

SEQ ID NO: 24 - DNA, 15 bp, Artificial Sequence
  HIV-1 target sequence
tgggagctctctggc

SEQ ID NO: 25 - DNA, 17 bp, Artificial Sequence
  U1 snRNA target-annealing sequence
atgccagagagctccca

SEQ ID NO: 26 - DNA, 15 bp, Artificial Sequence
  HIV-1 target sequence
taaagcttgccttga

SEQ ID NO: 27 - DNA, 17 bp, Artificial Sequence
  U1 snRNA target-annealing sequence
attcaaggcaagcttta

SEQ ID NO: 28 - DNA, 15 bp, Artificial Sequence
  HIV-1 target sequence
tagagatccctcaga

SEQ ID NO: 29 - DNA, 17 bp, Artificial Sequence
  U1 snRNA target-annealing sequence
attctgagggatctcta

SEQ ID NO: 30 - DNA, 9 bp, Artificial Sequence
  HIV-1 target sequence
gcagtggcg

SEQ ID NO: 31 - DNA, 11 bp, Artificial Sequence
  U1 snRNA target-annealing sequence
atcgccactgc

SEQ ID NO: 32 - DNA, 15 bp, Artificial Sequence
  HIV-1 target sequence
agtggcgcccgaaca

SEQ ID NO: 33 - DNA, 17 bp, Artificial Sequence
  U1 snRNA target-annealing sequence
attgttcgggcgccact

SEQ ID NO: 34 - DNA, 15 bp, Artificial Sequence
  HIV-1 target sequence
gggacttgaaagcga

SEQ ID NO: 35 - DNA, 17 bp, Artificial Sequence
  U1 snRNA target-annealing sequence
attcgctttcaagtccc

SEQ ID NO: 36 - DNA, 15 bp, Artificial Sequence
  HIV-1 target sequence
aagggaaaccagagg

SEQ ID NO: 37 - DNA, 17 bp, Artificial Sequence
  U1 snRNA target-annealing sequence
atcctctggtttccctt

SEQ ID NO: 38 - DNA, 15 bp, Artificial Sequence
  HIV-1 target sequence
agctctctcgacgca

SEQ ID NO: 39 - DNA, 17 bp, Artificial Sequence
  U1 snRNA target-annealing sequence
attgcgtcgagagagct

SEQ ID NO: 40 - DNA, 15 bp, Artificial Sequence
  HIV-1 target sequence
ggactcggcttgctg

SEQ ID NO: 41 - DNA, 17 bp, Artificial Sequence
  U1 snRNA target-annealing sequence
atcagcaagccgagtcc

SEQ ID NO: 42 - DNA, 15 bp, Artificial Sequence
  HIV-1 target sequence
aagcgcgcacggcaa

SEQ ID NO: 43 - DNA, 17 bp, Artificial Sequence
  U1 snRNA target-annealing sequence
atttgccgtgcgcgctt

SEQ ID NO: 44 - DNA, 15 bp, Artificial Sequence
  HIV-1 target sequence
gaggcgaggggcggc

SEQ ID NO: 45 - DNA, 17 bp, Artificial Sequence
  U1 snRNA target-annealing sequence
atgccgcccctcgcctc

SEQ ID NO: 46 - DNA, 15 bp, Artificial Sequence
  HIV-1 target sequence
gactggtgagtacgc

SEQ ID NO: 47 - DNA, 17 bp, Artificial Sequence
  U1 snRNA target-annealing sequence
atgcgtactcaccagtc

SEQ ID NO: 48 - DNA, 11 bp, Artificial Sequence
  HIV-1 target sequence
aattttgacta

SEQ ID NO: 49 - DNA, 11 bp, Artificial Sequence
  U1 snRNA target-annealing sequence
atgtcaaaatt

SEQ ID NO: 50 - DNA, 15 bp, Artificial Sequence
  HIV-1 target sequence
aattttgactagcgg

SEQ ID NO: 51 - DNA, 17 bp, Artificial Sequence
  U1 snRNA target-annealing sequence
atccgctagtcaaaatt

SEQ ID NO: 52 - DNA, 15 bp, Artificial Sequence
  HIV-1 target sequence
gcggaggctagaagg

SEQ ID NO: 53 - DNA, 17 bp, Artificial Sequence
  U1 snRNA target-annealing sequence
atccttctagcctccgc

SEQ ID NO: 54 - DNA, 15 bp, Artificial Sequence
  HIV-1 target sequence
agagagatgggtgcg

SEQ ID NO: 55 - DNA, 17 bp, Artificial Sequence
  U1 snRNA target-annealing sequence
atcgcacccatctctct

SEQ ID NO: 56 - DNA, 15 bp, Artificial Sequence
  HIV-1 target sequence
agagcgtcggtatta

SEQ ID NO: 57 - DNA, 17 bp, Artificial Sequence
  U1 snRNA target-annealing sequence
attaatactgacgctct

SEQ ID NO: 58 - DNA, 15 bp, Artificial Sequence
  HIV-1 target sequence
agcgggggagaatta

SEQ ID NO: 59 - DNA, 17 bp, Artificial Sequence
  U1 snRNA target-annealing sequence
attaattctcccccgct

SEQ ID NO: 60 - DNA, 15 bp, Artificial Sequence
  HIV-1 target sequence
gatcgcgatgggaaa

SEQ ID NO: 61 - DNA, 17 bp, Artificial Sequence
  U1 snRNA target-annealing sequence
attttcccatcgcgatc

SEQ ID NO: 62 - DNA, 15 bp, Artificial Sequence
  HIV-1 target sequence
aaattcggttaaggc

SEQ ID NO: 63 - DNA, 17 bp, Artificial Sequence
  U1 snRNA target-annealing sequence
atgccttaaccgaattt

SEQ ID NO: 64 - DNA, 15 bp, Artificial Sequence
  HIV-1 target sequence
gatcttcagacctgg

SEQ ID NO: 65 - DNA, 17 bp, Artificial Sequence
  U1 snRNA target-annealing sequence
atccaggtctgaagatc

SEQ ID NO: 66 - DNA, 15 bp, Artificial Sequence
  HIV-1 target sequence
ttacacaagcttaat

SEQ ID NO: 67 - DNA, 17 bp, Artificial Sequence
  U1 snRNA target-annealing sequence
atattaagcttgtgtaa

SEQ ID NO: 68 - DNA, 15 bp, Artificial Sequence
  HIV-1 target sequence
tagtagacataatag

SEQ ID NO: 69 - DNA, 17 bp, Artificial Sequence
  U1 snRNA target-annealing sequence
atctattatgtctacta

SEQ ID NO: 70 - RNA, 164 nt, Artificial Sequence
  U1 snRNA molecule
auacuuaccuggcaggggagauaccaugaucacgaaggugguuuucccagggcgaggcuuauccauugcacuccggaugugcugaccccugcgauuuccccaaaugugggaaacucuacugcauaauuugugguagugggggacugcguucgcgcuuuccccug